(19) United States
(12) Patent Application Publication
(10) Pub. No.: US 2025/0282723 A1
(43) Pub. Date: Sep. 11, 2025

(54) AMYLOID TARGETING AGENTS AND METHODS OF USING THE SAME

(71) Applicant: The Regents of the University of California, Oakland, CA (US)

(72) Inventor: Jerry YANG, La Jolla, CA (US)

(21) Appl. No.: 18/567,316

(22) PCT Filed: Jun. 15, 2022
(86) PCT No.: PCT/US2022/033610

§ 371 Date: Dec. 5, 2023

Related U.S. Application Data
(60) Provisional application No. 63/211,362, filed on Jun. 16, 2021.

(51) Int. Cl. C07D 211/14; A61K 31/4458

(52) U.S. Cl. CPC C07D 211/14; A61K 31/4458

(57) ABSTRACT

Described herein, inter alia, are molecular rotor fluorophores useful for detection of amyloid or amyloid like proteins, and methods for treating diseases associated with an amyloid or amyloid like proteins.

### CROSS-REFERENCES TO RELATED APPLICATIONS

This application claims the benefit of U.S. Provisional Application No. 63/211,362, filed Jun. 16, 2021, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. AG062362 awarded by the National Institutes of Health. The government has certain rights in the invention.

### BACKGROUND

Amyloid plaque accumulation in the brain is the hallmark of many neurodegenerative disorders, including Alzheimer's disease (AD), Parkinson disease, Down's syndrome and Creutzfeldt-Jakob disease (CJD). Approaches to clinically diagnose and monitor the progression of these diseases include targeting of amyloid deposits with small-molecule imaging agents. Accordingly, fluorescence-based small molecule imaging of amyloids is a low cost, accessible, and non-radioactive technique for to detection of the amyloid deposits. Fluorescent compounds that maintain their brightness, spectroscopic properties, and specificity for binding amyloids in neuronal tissue, and exhibit superior chemical/hydrolytic stability in physiologically relevant solutions are disclosed herein. The enhanced stability of such compounds is useful in labeling amyloid deposits in living systems. Disclosed herein, inter alia, are solutions to these and other problems in the art.

### BRIEF SUMMARY

In an aspect is provided a compound having the formula:

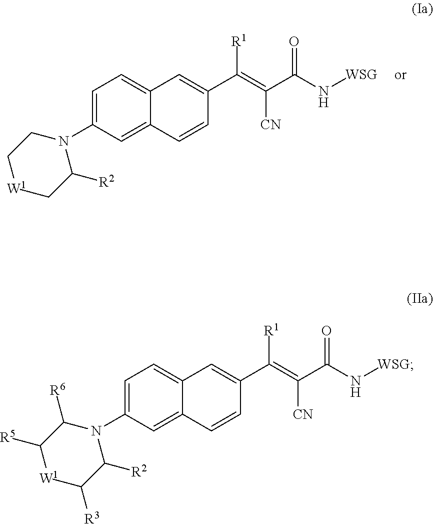

or a pharmaceutically acceptable salt thereof.

WSG is a water soluble group.

W¹ is O, N(R¹⁴), or C(R⁴)₂.

R¹⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹, R², R³, R⁴, R⁵, and R⁶ are each independently hydrogen, halogen, —CX¹₃, —CHX¹₂, —CH₂X¹, —OCX¹₃, —OCH₂X¹, —OCHX¹₂, —SOₙ₁R^{1A}, —SOᵥ₁NR^{1A}R^{1B}, —CN, —C(O)R^{1A}, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, —N₁HNR^{1A}R^{1B}, —NR^{1A}SO₂R^{1B}, —NR^{1A}C(O)R^{1B}, —NR^{1A}C(O)OR^{1B}, NR^{1A}OR^{1B}, N₃ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X¹ is —F, —Cl, —Br, or —I.

The symbol n1 is independently an integer from 0 to 4. The symbol m1 is independently 1 or 2. The symbol v1 is independently 1 or 2.

In an aspect is provided a pharmaceutical composition including a compound described herein, or a pharmaceutically acceptable salt thereof, and a pharmaceutically acceptable excipient.

In an aspect is provided a method for detecting one or more amyloid or amyloid like proteins comprising contacting a compound according to according to any one of Formula Ia or Formula IIa (e.g., Formula I or Formula II) or a pharmaceutical composition thereof with a sample potentially comprising the amyloid or amyloid like protein, wherein in presence of an amyloid or amyloid like protein the compound forms a detectable complex, detecting the formation of the detectable complex such that the presence or absence of the detectable complex correlates with the presence or absence of the amyloid or amyloid like protein.

In an aspect is provided a method of determining the presence or absence of one or more disease or condition in a subject comprising administering to the subject an effective amount of a compound according to any one of Formula Ia or Formula IIa (e.g., Formula I or Formula II) or a pharmaceutical composition thereof, wherein in presence of the disease or condition the administered compound forms a detectable complex, and detecting the formation of the detectable complex such that presence or absence of detectable complex correlates with the presence or absence of the disease or condition.

### BRIEF DESCRIPTION OF THE DRAWINGS

FIGS. 1A-IC illustrate examples of amyloid-binding molecular rotor fluorophores. FIG. 1A: Structure of ARCAM (1). FIG. 1B: 3D rendering of ARCAM highlighting potential rotatable bonds (a and b) between the electron donor and acceptor. WSG in FIGS. 1A-IC refers to water solubilizing group. FIG. 1C: Structures of Thioflavin T (ThT), Congo Red (CR) and new fluorescent ARCAM analogs 2-5.

FIG. 2 illustrates fluorescence emission spectra probes 1-5 (probes are interchangeably referred as analogs) free in aqueous solution (shown in black color) or in the presence of aggregated A3 (shown in grey color).

FIG. 3 illustrates representative absorbance vs emission curves for ARCAM (1) and Coumarin 30. The graph on the left represents Absorbance at Excitation Wavelength vs. Integrated Fluorescence Intensity (F.I.) graph for Coumarin 30 (concentrations used=0.096, 0.072, 0.048, 0.024, 0 mM). The graph on the right represents Absorbance vs Integrated Emission graph for ARCAM (1) (concentrations used=0.0125, 0.01, 0.0075, 0.005, 0 mM).

FIG. 4 illustrates representative absorbance vs emission curves for Compound 2 and Coumarin 30. The graph on the left represents Absorbance at Excitation Wavelength vs. Integrated Fluorescence Intensity (F.I.) graph for Coumarin 30 (concentrations used=0.12, 0.096, 0.072, 0.048, 0.024, 0 mM). The graph on the right represents Absorbance vs Integrated Emission graph for Compound 2 (concentrations used=0.19, 0.152, 0.114, 0.076, 0.038, 0 mM).

FIG. 5 illustrates representative absorbance vs emission curves for Compound 3 and Coumarin 30. The graph on the left represents Absorbance at Excitation Wavelength vs. Integrated Fluorescence Intensity (F.I) graph for Coumarin 30 (concentrations used=0.096, 0.072, 0.048, 0.024, 0 mM). The graph on the right represents Absorbance vs Integrated Emission graph for Compound 3 (concentrations used=0.1, 0.06, 0.04, 0.02, 0 mM).

FIG. 6 illustrates representative absorbance vs emission curves for Compound 4 and Coumarin 30. The graph on the left represents Absorbance at Excitation Wavelength vs. Integrated Fluorescence Intensity (F.I.) graph for Coumarin 30 (concentrations used=0.12, 0.072, 0.048, 0.024, 0 mM). The graph on the right represents Absorbance vs Integrated Emission graph for Compound 4 (concentrations used=0.02, 0.016, 0.012, 0.004, 0 mM).

FIG. 7 illustrates representative absorbance vs emission curves for Compound 5 and Coumarin 30. The graph on the left represents Absorbance at Excitation Wavelength vs. Integrated Fluorescence Intensity (F.I.) graph for Coumarin 30 (concentrations used=0.12, 0.096, 0.072, 0.048, 0.024, 0 mM). The graph on the right represents Absorbance vs Integrated Emission graph for Compound 5 (concentrations used=0.08, 0.064, 0.048, 0.032, 0.016, 0 mM).

FIG. 8 illustrates representative absorbance vs emission curves for ThT and Coumarin 30. The graph on the left represents Absorbance at Excitation Wavelength vs. Integrated Fluorescence Intensity (F.I) graph for Coumarin 30 (concentrations used=0.096, 0.072, 0.048, 0.024, 0 mM). The graph on the right represents Absorbance vs Integrated Emission graph for ThT (concentrations used=0.048, 0.036, 0.024, 0.012, 0 mM).

FIG. 9 shows the normalized emission spectra of compounds 1-5 in the presence of absence of aggregated Aβ in deionized water.

FIGS. 10A-10E illustrate binding affinity estimations for compounds 1-5 (FIG. 10A-10E, respectively). Measurements were performed in independent triplicates and averaged; error bars indicate standard deviation, where visible. KD values were obtained from this data as previous reported²¹.

FIG. 11 shows the fluorescence signal of thioflavin T (ThT) in the presence or absence of aggregated synthetic Aβ. This analysis is conventionally used to support that the Aβ sample is highly aggregated into amyloid. This amyloid sample was subsequently used to characterize the fluorescence response of compounds 1-5 (FIG. 9) in the presence of aggregated Aβ in solution.

FIG. 12 shows an SDS-PAGE gel of aggregated Aβ(1-42) separated on the NuPAGE 4-12% bis-tris gel, and stained by Pierce Silver Stain Kit. The abundance fibrils, oligomers, and monomers was estimated using ImageJ by multiplying the average intensity of each region by the area of each region and dividing by the total intensityxarea of all 3 bands.

FIGS. 13A-13B. The general structure of aryl cyano amides represented by compounds 1-5 and the effects of small aliphatic substituents located at the X and Y position on relative fluorescence signal when bound to aggregated Aβ in solution.

FIG. 14. Structure of select naphthalenyl cyano amide (NAPHTHCAM) probes.

FIGS. 15A-15B. Representative images of binding regions and orientations for NAPHTHCAM probes in Aβ (FIG. 15A) and αS (FIG. 15B). View down the barrel for Aβ (FIG. 15A, bottom panel) and αS (FIG. 15B, bottom panel), where the dashed circles represent the length of each 2-piperidinyl substituent.

FIGS. 16A-16G. Fluorescence emission spectra of probes 1-4 (FIGS. 16A-16D, respectively) from FIG. 14; free, bound to αS fibrils, and bound to Aβ fibrils. Normalizations are set to Emₘₐₓ(free)=1. Fluorescence enhancement of probes 1-4 with αS fibrils (FIG. 16E) and Aβ fibrils (FIG. 16F). FIG. 16G: Selectivity of fluorescence response of probes 1-4 to αS fibrils over Aβ fibrils. Asterisks indicate statistically significant differences by One-Way ANOVA compared to NAPHTHCAM-H (**p<0.01, *p<0.05).

FIG. 17. Synthetic route for the preparation of NAPHTHCAM-Pr fluorescent probes.

FIGS. 18A-18C. Tissue images and quantifications for probes 1 and 3 from FIG. 14. FIG. 18A: Images of probes in human AD frontal cortex tissue, scale bars 20 μm (top) and images of probes in human PD frontal cortex tissue, major scale bars 10 μm, inset scale bars 3 μm (bottom). FIG. 18B: Quantification of signal to background of plaques in AD tissue. FIG. 18C: Quantification of signal to background of deposits in PD tissue. Asterisks indicate statistically significant differences by One-Way ANOVA between probes (****p<0.0001 ***p<0.001, **p<0.01, *p<0.05).

FIG. 19. Structure of Aβ fibril (PDB ID: 2MXU) outlining location and residues of binding site 1.

FIG. 20. Structure of αS fibril (PDB ID: 2NOA) outlining location and residues of binding sites 1, 2, 3, and 4.

FIGS. 21A-21D. Docked poses of probes 1 (FIG. 21A), 2 (FIG. 21B), 3 (FIG. 21C), and 4 (FIG. 21D) from FIG. 14 in Site 1 of Aβ fibrils.

FIGS. 22A-22D. Docked poses of probes 1 (FIG. 22A), 2 (FIG. 22B), 3 (FIG. 22C), and 4 (FIG. 22D) from FIG. 14 in Site 4 of αS fibrils.

FIGS. 23A-23B. Representative absorbance vs. emission curves for Coumarin 30 (FIG. 23A) and NAPHTHCAM-Pr (4) (FIG. 23B) from technical triplicates. FIG. 23A: Absorbance at Excitation Wavelength vs. Integrated Fluorescence Intensity (F.I.) graph for Coumarin 30 (concentrations used=0.12, 0.096, 0.072, 0.048, 0.024, 0 mM). FIG. 23B: Absorbance vs Integrated Emission graph for NAPHTHCAM-Pr (4) (concentrations used=0.03125, 0.025, 0.01875, 0.0125, 0.00625, 0 mM).

FIG. 24. Fluorescence emission spectrum of Thioflavin T (ThT) with aggregated α-synuclein (top curve) and without aggregated α-synuclein (bottom curve).

FIG. 25. Quantification using ImageJ software comparing the signal to background of deposits in the PD Substantia Nigra (SN) and Hippocampus (Hip) for NAPHTHCAM-H & NAPHTHCAM-Et.

FIGS. 26A-26B. FIG. 26A: Comparison for each probe signal to background from staining in AD (first bar in each set) human FC and PD (second bar in each set) human FC. Asterisks indicate statistically significant differences by One-Way ANOVA (***p<0.001, **p<0.01, *p<0.05). FIG. 26B: Selective fluorescence response in tissue towards αS deposits compared to Aβ plaques for each probe.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In aspects, treating is preventing. In aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is not prophylactic treatment.

“Patient” or “subject” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, and other non-mammalian animals. In aspects, a patient is human.

An “effective amount,” as used herein, is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). In these methods, the effective amount of the compounds described herein is an amount effective to accomplish the stated purpose of the method. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. For any compound described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in addition to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In aspects, monocyclic ring systems are cyclic hydrocarbon groups containing from 3 to 8 carbon atoms, where such groups can be saturated or unsaturated, but not aromatic. In aspects, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In aspects, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]-heptane, bicyclo[2.2.1]heptane, bicyclo[2.2.2]octane, bicyclo[3.2.2]nonane, bicyclo[3.3.1]nonane, and bicyclo[4.2.1]nonane. In aspects, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkyl is attached to the parent molecular moiety through any carbon atom contained within the monocyclic cycloalkyl ring. In aspects, cycloalkyl groups are optionally substituted with one or two groups which are independently oxo or thia. In aspects, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkyl is attached to the parent molecular moiety through any carbon atom contained within the base ring. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In aspects, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In aspects, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In aspects, bicyclic cycloalkenyl rings are bridged monocyclic rings or a fused bicyclic rings. In aspects, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In aspects, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkenyl is attached to the parent molecular moiety through any carbon atom contained within the monocyclic cycloalkenyl ring. In aspects, cycloalkenyl groups are optionally substituted with one or two groups which are independently oxo or thia. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkenyl is attached to the parent molecular moiety through any carbon atom contained within the base ring. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together or multiple spirocyclic rings wherein at least one of the fused or spirocyclic rings is a heterocycloalkyl ring and wherein the multiple rings are attached to the parent molecular moiety through any atom contained within a heterocycloalkyl ring of the multiple rings.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydro-benzofuran-2-yl, 2,3-dihydrobenzofuran-3-yl, indolin-1-yl, indolin-2-yl, indolin-3-yl, 2,3-dihydro-benzothien-2-yl, decahydroquinolinyl, decahydroisoquinolinyl, octahydro-1H-indolyl, and octahydrobenzofuranyl. In aspects, heterocyclyl groups are optionally substituted with one or two groups which are independently oxo or thia. In certain aspects, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In aspects, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the multiple rings are attached to the parent molecular moiety through any atom contained within a heteroaromatic ring of the multiple rings. A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “˜” and “-” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In aspects, the alkylarylene group has the formula:

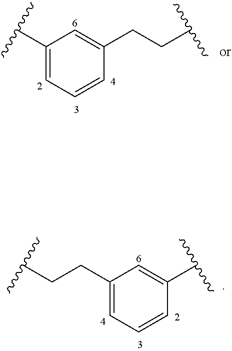 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In aspects, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =O, =NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In embodiments, each substituted group described in the compounds herein is substituted with at least one substituent group. In aspects, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In aspects, at least one or all of these groups are substituted with at least one size-limited substituent group. In aspects, at least one or all of these groups are substituted with at least one lower substituent group.

In embodiments, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In aspects, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some aspects, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In aspects, the compound (e.g., nucleotide analogue) is a chemical species set forth in the Examples section, claims, aspects, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In aspects, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In aspects, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group is optionally different. In aspects, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group is optionally different. In aspects, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group is optionally different. In aspects, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW2}.

Finally, each second substituent group (e.g., R^{1.2}, R^{2.2}, R^{3.2}, R^{4.2}, R^{5.2} R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5} . . . R^{100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

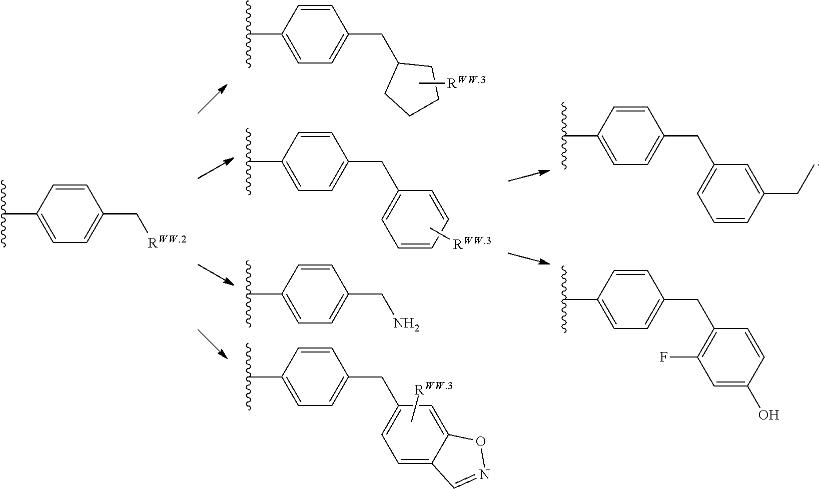

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NH₄NH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2} substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NI—N₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3} substituted or unsubstituted aryl (e.g., C₆-C₁₂, C₆-C₁₀, or phenyl), or R^{LWW.3} substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-Cₛ, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂—CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}_substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the disclosure do not include those that are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “about” or “approximately” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “pharmaceutically acceptable salts” is meant to include salts of the active compounds that are prepared with relatively nontoxic acids or bases, depending on the particular substituents found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds contain both basic and acidic functionalities that allow the compounds to be converted into either base or acid addition salts.

The compounds may exist as salts, such as with pharmaceutically acceptable acids. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the disclosure provides compounds which are in a prodrug form. Prodrugs of the compounds described herein are those compounds that readily undergo chemical changes under physiological conditions to provide the compounds disclosed herein. Prodrugs of the compounds described herein may be converted in vivo after administration. Additionally, prodrugs can be converted to the compounds of the disclosure by chemical or biochemical methods in an ex vivo environment, such as, for example, when contacted with a suitable enzyme or chemical reagent.

Certain compounds can exist in unsolvated forms as well as solvated forms, including hydrated forms. In general, the solvated forms are equivalent to unsolvated forms and are encompassed within the scope of the disclosure. Certain compounds may exist in multiple crystalline or amorphous forms. In general, all physical forms are equivalent for the uses contemplated by disclosure and are intended to be within the scope of the disclosure.

In embodiments, diseases or conditions that are treated with the compounds of the present disclosure include diseases or conditions accompanied by protein that produces amyloid like morphology and disease or conditions associated with the formation of abnormal protein structures, protein aggregation, or protein misfolding. In embodiments, an abnormal protein structure is a protein structure that arises when a protein or peptide refolds from the three-dimensional structure, which it generally adopts in healthy individuals, into a different three-dimensional structure, which is associated with a pathological condition. In embodiments, diseases or conditions that are treated with the compounds of the present disclosure are diseases or conditions associated with amyloid or amyloid-like proteins. In embodiments, such diseases are referred to as amyloid based diseases or conditions. Amyloid based diseases or conditions, include any disease or condition that is associated with amyloid or amyloid-like protein and is characterized, in part, by the buildup of extracellular deposits of amyloid or amyloid-like material. In the context of this disclosure, amyloid based diseases or conditions also include disease or conditions accompanied by protein that produces amyloid like morphology. These diseases include, but are not limited to, neurological disorders such as Alzheimer's disease (AD), Parkinson's disease, Huntington's disease, diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex. Other diseases which are based on or associated with amyloid-like proteins are progressive supranuclear palsy, multiple sclerosis; Creutzfeldt Jacob disease, Parkinson's disease, HIV-related dementia, ALS (amyotropic lateral sclerosis), inclusion-body myositis (IBM), Adult Onset Diabetes; senile cardiac amyloidosis; endocrine tumors, and other diseases, including amyloid-associated ocular diseases that target different tissues of the eye, such as the visual cortex, including cortical visual deficits; the anterior chamber and the optic nerve, including glaucoma; the lens, including cataract due to beta-amyloid deposition; the vitreous, including ocular amyloidosis; the retina, including primary retinal degenerations and macular degeneration, in particular age-related macular degeneration; the optic nerve, including optic nerve drusen, optic neuropathy and optic neuritis; and the cornea, including lattice dystrophy.

The term “amyloid protein” is intended to denote a protein which is involved in the formation of fibrils, plaques and/or amyloid deposits, either by being part of the fibrils, plaques and/or deposits as such or by being part of the biosynthetic pathway leading to the formation of the fibrils, plaques and/or amyloid deposits. In the present context the term “protein” or is intended to mean both short peptides of from 2 to 10 amino acid residues, oligopeptides of from 11 to 100 amino acid residues, polypeptides of more than 100 amino acid residues, and full length proteins. The terms also encompass peptides having substantial similarity to amyloid proteins, such as, e.g., structural variants. In embodiments, the proteins occur naturally or be synthetically constructed. The term amyloid protein or amyloid like protein also includes amyloidgenic proteins and proteins that produce amyloid like morphology.

The term “amyloid beta” or “Ap” refers to proteins that are the main component of amyloid plaques found in the brains of patients with Alzheimer's disease. In embodiments, the amyloid beta proteins are formed from the amyloid precursor protein (APP), which is cleaved by beta secretase and gamma secretase to form Ap.

The term “alpha-synuclein” refers to a protein that regulates synaptic vesicle trafficking and neutotransmitter release. In embodiments, the alpha-synuclein protein encoded by the SNCA gene has the amino acid sequence set forth in or corresponding to Entrez 6622, UniProt P37840, RefSeq (protein) NP_000336.1, RefSeq (protein) NP_001139526.1, RefSeq (protein) NP_001139527.1, or RefSeq (protein) NP_009292.1, or homolog thereof. In embodiments, the amino acid sequence or nucleic acid sequence is the sequence known at the time of filing of the present application.

The term “substantial similarity” means that two peptide sequences, when optimally aligned, share at least 50% sequence identity, or at least 60% sequence identity, or at least 70% sequence identity, or at least 80% sequence identity, or at least 90 percent sequence identity, or at least 95 percent sequence identity or more (e.g., 99% sequence identity). Preferably, residue positions, which are not identical, differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. In embodiments, residue positions, which are not identical are also composed of peptide analogs, including unnatural amino acids or derivatives of such. Analogs typically differ from naturally occurring peptides at one, two or a few positions, often by virtue of conservative substitutions. Some analogs also include unnatural amino acids or modifications of N or C terminal amino acids at one, two or a few positions. Examples of unnatural amino acids are D-amino acids, alpha, alpha-disubstituted amino acids, N-alkyl amino acids, lactic acid, 4-hydroxyproline, y-carboxyglutamate, epsilon-N,N,N-trimethyllysi-ne, epsilon-N-acetyllysine, O-phosphoserine, N-acetylserine, N-formylmethionine, 3-methylhistidine, 5-hydroxylysine, omega.-N-methylarginine, and isoaspartic acid.

### II. Overview

In embodiments, the disclosure provides novel compounds that are employed in the detection, diagnosis, treatment, and monitoring of diseases or conditions associated with protein aggregation or protein misfolding. In embodiments, the compounds of the disclosure are also employed in the detection, diagnosis, treatment, and monitoring of amyloid based diseases or conditions. The disclosure further provides pharmaceutical compositions comprising these compounds and the use of these compounds for the preparation of medicaments for the treatment of such diseases or conditions.

The compounds of the disclosure are designed form a detectable complex in presence of an amyloid or amyloid-like protein. In embodiments, the compounds disclosed herein are classified as molecular rotor fluorophores. The compounds comprise an electron rich donor moiety covalently connected to a conjugated pi system (for example, to an aromatic pi network) and in electronic conjugation to an electron poor acceptor moiety covalently connected elsewhere on the pi system. The compounds also comprise one or more single bonds between the donor and acceptor that can rotate freely under standard thermal control of the environment in the temperature range of interest. The rotation of the single bond allows the donor and acceptor to remain substantially decoupled in the absence of binding to a protein and thus the compounds exhibit poor fluorescence signal. However, in presence of an amyloid or amyloid-like protein these compounds may bind to amyloid or amyloid-like proteins. Accordingly, the rotatable single bonds may become essentially frozen and the electronic coupling between the donor and acceptor may be substantially enhanced. In embodiments, it leads to a strong fluorescence signal upon irradiation with an appropriate wavelength of light. In embodiments, the strong fluorescence enhancement of these compounds upon binding to amyloid or amyloid-like proteins compared to the free compound in solution results in excellent signal to noise ratio and make it possible to image amyloid or amyloid-like proteins with high sensitivity.

Also provided herein is a method for detecting an amyloid or amyloid-like protein. The method comprises contacting a compound of the disclosure or a pharmaceutical composition thereof with the sample potentially comprising the amyloid or amyloid-like protein, wherein in presence of an amyloid or amyloid-like protein the compound forms a detectable complex, and detecting the formation of the detectable complex such that the presence or absence of the detectable complex correlates with the presence or absence of the amyloid or amyloid like protein. In embodiments, the detection of the detectable complex in the methods of the disclosure comprises illuminating the sample with light of an appropriate wavelength and detecting light received from the sample. In embodiments, the wavelength of the illuminating light is varied and selected according to the fluorescence excitation and emission spectrum of the detectable complex. In embodiments, the detectable complex has a fluorescent excitation peak in the range of 350-500 nm, and the fluorescence emission spectrum of the detectable complex in the range of 500-550 nm. In embodiments, the illuminating light has a wavelength of 350-450 nm (example 400 nm). In embodiments, amyloid or amyloid like protein or peptide is detected by the methods of the disclosure. In embodiments, the method is used to detect the presence or absence of Aβ peptide, prion peptide, alpha-synuclein, or superoxide dismutase.

The disclosure also provides a method of determining the presence or absence of one or more disease or condition in a subject. The method comprises administering to the subject an effective amount of a compound of the disclosure or a pharmaceutical composition thereof, wherein in presence of the a disease or condition the administered compound forms a detectable complex, and detecting the formation of the detectable complex such that presence or absence of the detectable complex correlates with the presence or absence of the disease or condition. In embodiments, the method includes comparing the amount of the detectable complex to a normal control value, wherein an increase in the amount of the detectable complex compared to a normal control value indicates that said patient is suffering from or is at risk of developing the disease or condition. Also provided herein is a method of treating, preventing or alleviating the symptoms of a disease or condition in a subject. The method comprises administering to a subject in need of treatment an effective amount of a compound of the disclosure or a pharmaceutical composition thereof. In embodiments, the subject is a mammal. In some case, the subject is a primate (such as a human), canine, feline, ovine, bovine and the like.

In embodiments, the disease or condition is a disease or condition characterized by protein aggregation or misfolding. In embodiments, the disease or condition is also an amyloid based disease or condition. In embodiments, the amyloid-based disease or condition is any disease or condition associated with the increased or decreased presence of amyloid or amyloid like proteins, such as the presence of amyloid plaques or other amyloid aggregates. In embodiments, the disease is a neuronal disease. In embodiments, the disease is a neurodegenerative diseases, in which amyloid-beta peptides, oligomers, fibrils, or plaques are implicated. In embodiments, the disease is Alzheimer's disease, Parkinson's disease, Huntington's disease, amyotrophic lateral sclerosis (ALS), Lewy body dementia (LBD), or Down's syndrome. In embodiments, the amyloid-based disease or condition also includes ocular diseases associated with pathological abnormalities/changes in the tissues of the visual system, particularly associated with amyloid-beta-related pathological abnormalities/changes in the tissues of the visual system, such as, for example, neuronal degradation. In embodiments, pathological abnormalities occur, for example, in different tissues of the eye, such as the visual cortex leading to cortical visual deficits; the anterior chamber and the optic nerve leading to glaucoma; the lens leading to cataract due to beta-amyloid deposition; the vitreous leading to ocular amyloidosis; the retina leading to primary retinal degeneration and macular degeneration; the optic nerve leading to optic nerve drusen, optic neuropathy and optic neuritis; and the cornea leading to lattice dystrophy.

In embodiments, the compounds and the methods of the disclosure are also used to monitor minimal residual disease in a patient following treatment with a compound or a mixture according to the disclosure. In embodiments, a sample or a specific body part or body area suspected to contain the amyloid antigen is contacted with a compound of the disclosure, and the compound is allowed to bind to the amyloid or amyloid like protein to form a detectable complex. In embodiments, the formation of the detectable complex is detected and its presence or absence is correlated with the presence or absence of amyloid or amyloid like protein in the sample or specific body part or area. In embodiments, the amount of said detectable complex is compared to a normal control value, wherein an increase in the amount of said detectable complex compared to a normal control value indicates that the patient may still be suffering from a minimal residual disease.

In embodiments, the compounds and methods disclosed herein are useful for predicting responsiveness of a patient to a treatment. In embodiments, a sample or a specific body part or body area suspected to contain the amyloid or amyloid like protein is brought into contact with a compound of the disclosure, such that in presence of the amyloid or amyloid like protein the compound binds to the amyloid or amyloid like protein to form a detectable complex. In embodiments, the formation of the detectable complex is detected and the presence or absence of the detectable complex is correlated with the presence or absence of amyloid or amyloid like protein in the sample or specific body part or area. In embodiments, the amount of the detectable complex before and after onset of the treatment is compared, such that a decrease in the amount of the detectable complex indicates that the patient is being responsive to the treatment.

### III. Compounds

In an aspect is provided a compound having the formula:

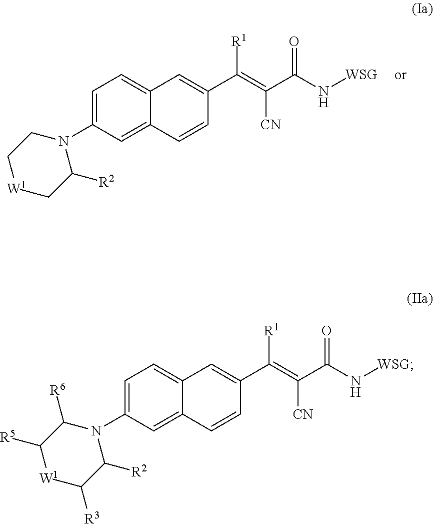

or a pharmaceutically acceptable salt thereof.
WSG is a water soluble group.
W¹ is O, N(R¹⁴), or C(R⁴)₂.
R¹⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
R¹, R², R³, R⁴, R⁵, and R⁶ are each independently hydrogen, halogen, —CX¹₃, —CHX¹₂, —CH₂X¹, —OCX¹₃, —OCH₂X¹, —OCHX¹₂, SOₙ₁R^{1A}, —SOᵥ₁NR^{1A}R^{1B}, —CN, —C(O)R^{1A}, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, —NHNR^{1A}R^{1B}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
X¹ is —F, —Cl, —Br, or —I.

The symbol n1 is independently an integer from 0 to 4.

The symbol m1 is independently 1 or 2.

The symbol v1 is independently 1 or 2.

In embodiments, the compound has the formula:

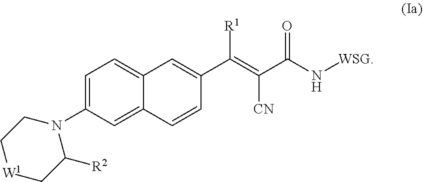

WSG, W¹, R¹, and R² are as described herein, including in embodiments.

In embodiments, the compound has the formula:

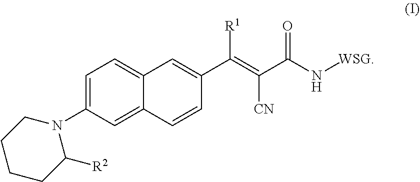

WSG, R¹, and R² are as described herein, including in embodiments.

In embodiments, the compound has the formula:

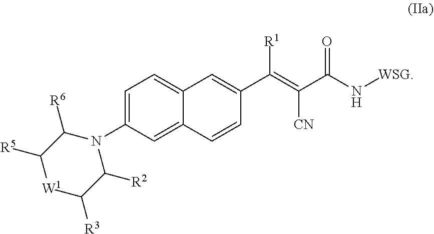

WSG, W¹, R¹, R², R³, R⁵, and R⁶ are as described herein, including in embodiments.

In embodiments, the compound has the formula:

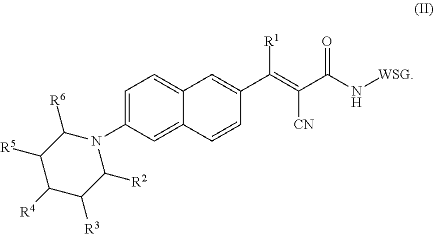

WSG, R¹, R², R³, R⁴, R⁵, and R⁶ are as described herein, including in embodiments.

In an aspect is provided compounds of Formula (I), pharmaceutically acceptable salts of the compound of Formula (I), compounds of Formula (II), and pharmaceutically acceptable salts of the compound of Formula (II):

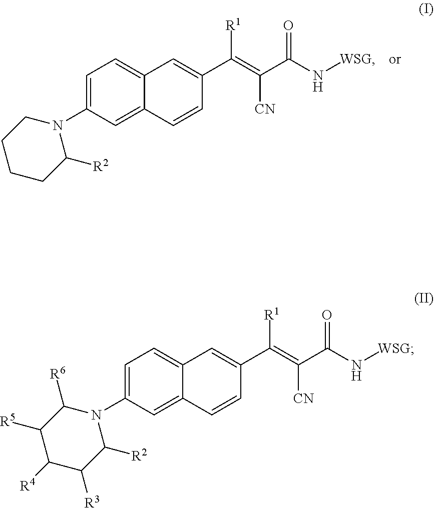

wherein WSG is a water soluble group; R¹, R², R³, R⁴, R⁵, R⁶, and R⁷ are each independently hydrogen, halogen, —CX¹₃, —CHX¹₂, —CH₂X¹, —OCX¹₃, —OCH₂X¹, —OCHX¹₂, —SOₙ₁R^{1A}, —SOᵥ₁NR^{1A}R^{1B}, —CN, —C(O)RA, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, —NHNR^{1A}R^{1B}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹ is —F, —Cl, —Br, or —I; n1 is an integer from 0 to 4; and m1 is 1 or 2; and v1 is 1 or 2.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when RA is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R^{1B} is substituted, it is substituted with at least one size-limited substituent group. In embodiments, when R^{1A} is substituted, it is substituted with at least one lower substituent group.

In embodiments, R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1A} is independently hydrogen or unsubstituted C₁-C₄ alkyl. In embodiments, R^{1A} is independently hydrogen. In embodiments, R^{1A} is independently unsubstituted C₁-C₄ alkyl. In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted n-propyl. In embodiments, R^{1A} is independently unsubstituted isopropyl. In embodiments, R^{1A} is independently unsubstituted butyl. In embodiments, R^{1A} is independently unsubstituted n-butyl. In embodiments, R^{1A} is independently unsubstituted isobutyl. In embodiments, R^{1A} is independently unsubstituted tert-butyl.

In embodiments, R^{1B} is independently hydrogen or unsubstituted C₁-C₄ alkyl. In embodiments, R^{1B} is independently hydrogen. In embodiments, R^{1B} is independently unsubstituted C₁-C₄ alkyl. In embodiments, R^{1B} is independently unsubstituted methyl. In embodiments, R^{1B} is independently unsubstituted ethyl. In embodiments, R^{1B} is independently unsubstituted propyl. In embodiments, R^{1B} is independently unsubstituted n-propyl. In embodiments, R^{1B} is independently unsubstituted isopropyl. In embodiments, R^{1B} is independently unsubstituted butyl. In embodiments, R^{1B} is independently unsubstituted n-butyl. In embodiments, R^{1B} is independently unsubstituted isobutyl. In embodiments, R^{1B} is independently unsubstituted tert-butyl.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl. In embodiments, R¹ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

In embodiments, R′ is hydrogen. In embodiments, R¹ is halogen. In embodiments, R¹ is —F. In embodiments, R¹ is —Cl. In embodiments, R¹ is —Br. In embodiments, R¹ is —I. In embodiments, R¹ is —CCl₃. In embodiments, R¹ is —CBr₃. In embodiments, R¹ is —CF₃. In embodiments, R¹ is —CI₃. In embodiments, R¹ is —CH₂C₁. In embodiments, R¹ is —CH₂Br. In embodiments, R¹ is —CH₂F. In embodiments, R¹ is —CH₂I. In embodiments, R¹ is —CHCl₂. In embodiments, R¹ is —CHBr₂. In embodiments, R¹ is —CHF₂. In embodiments, R¹ is —CHI₂. In embodiments, R¹ is —CN. In embodiments, R¹ is —OH. In embodiments, R¹ is —NH₂. In embodiments, R¹ is —COOH. In embodiments, R¹ is —CONH₂. In embodiments, R¹ is —NO₂. In embodiments, R¹ is —SH. In embodiments, R¹ is —SO₃H. In embodiments, R¹ is —OSO₃H. In embodiments, R¹ is —SO₂NH₂. In embodiments, R¹ is —NHNH₂. In embodiments, R′ is —ONH₂. In embodiments, R′ is —NHC(O)NHNH₂. In embodiments, R′ is —OCCl₃. In embodiments, R¹ is —OCBr₃. In embodiments, R¹ is —OCF₃. In embodiments, R¹ is —OCl₃. In embodiments, R¹ is —OCH₂C₁. In embodiments, R¹ is —OCH₂Br. In embodiments, R¹ is —OCH₂F. In embodiments, R′ is —OCH₂I. In embodiments, R¹ is —OCHCl₂. In embodiments, R¹ is —OCHBr₂. In embodiments, R′ is —OCHF₂. In embodiments, R¹ is —OCHI₂. In embodiments, R¹ is hydrogen or unsubstituted C₁-C₄ alkyl. In embodiments, R¹ is unsubstituted C₁-C₄ alkyl. In embodiments, R¹ is unsubstituted methyl. In embodiments, R¹ is unsubstituted ethyl. In embodiments, R′ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

In embodiments, R² is hydrogen. In embodiments, R² is halogen. In embodiments, R² is —F. In embodiments, R² is —Cl. In embodiments, R² is —Br. In embodiments, R² is —I.

In embodiments, R² is —CCl₃. In embodiments, R² is —CBr₃. In embodiments, R² is —CF₃. In embodiments, R² is —CI₃. In embodiments, R² is —CH₂C₁. In embodiments, R² is —CH₂Br. In embodiments, R² is —CH₂F. In embodiments, R² is —CH₂I. In embodiments, R² is —CHCl₂. In embodiments, R² is —CHBr₂. In embodiments, R² is —CHF₂. In embodiments, R² is —CHI₂. In embodiments, R² is —CN. In embodiments, R² is —OH. In embodiments, R² is —NH₂. In embodiments, R² is —COOH. In embodiments, R² is —CONH₂. In embodiments, R² is —NO₂. In embodiments, R² is —SH. In embodiments, R² is —SO₃H. In embodiments, R² is —OSO₃H. In embodiments, R² is —SO₂NH₂. In embodiments, R² is —NHNH₂. In embodiments, R² is —ONH₂. In embodiments, R² is —NHC(O)NHNH₂. In embodiments, R² is —OCCl₃. In embodiments, R² is —OCBr₃. In embodiments, R² is —OCF₃. In embodiments, R² is —OCI₃. In embodiments, R² is —OCH₂C₁. In embodiments, R² is —OCH₂Br. In embodiments, R² is —OCH₂F. In embodiments, R² is —OCH₂I. In embodiments, R² is —OCHCl₂. In embodiments, R² is —OCHBr₂. In embodiments, R² is —OCHF₂. In embodiments, R² is —OCHI₂. In embodiments, R² is hydrogen or unsubstituted C₁-C₄ alkyl. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

In embodiments, R³ is hydrogen. In embodiments, R³ is halogen. In embodiments, R³ is —F. In embodiments, R³ is —Cl. In embodiments, R³ is —Br. In embodiments, R³ is —I. In embodiments, R³ is —CCl₃. In embodiments, R³ is —CBr₃. In embodiments, R³ is —CF₃. In embodiments, R³ is —CI₃. In embodiments, R³ is —CH₂C₁. In embodiments, R³ is —CH₂Br. In embodiments, R³ is —CH₂F. In embodiments, R³ is —CH₂I. In embodiments, R³ is —CHCl₂. In embodiments, R³ is —CHBr₂. In embodiments, R³ is —CHF₂. In embodiments, R³ is —CHI₂. In embodiments, R³ is —CN. In embodiments, R³ is —OH. In embodiments, R³ is —NH₂. In embodiments, R³ is —COOH. In embodiments, R³ is —CONH₂. In embodiments, R³ is —NO₂. In embodiments, R³ is —SH. In embodiments, R³ is —SO₃H. In embodiments, R³ is —OSO₃H. In embodiments, R³ is —SO₂NH₂. In embodiments, R³ is —NHNH₂. In embodiments, R³ is —ONH₂. In embodiments, R³ is —NHC(O)NHNH₂. In embodiments, R³ is —OCCl₃. In embodiments, R³ is —OCBr₃. In embodiments, R³ is —OCF₃. In embodiments, R³ is —OCl₃. In embodiments, R³ is —OCH₂C₁. In embodiments, R³ is —OCH₂Br. In embodiments, R³ is —OCH₂F. In embodiments, R³ is —OCH₂I. In embodiments, R³ is —OCHCl₂. In embodiments, R³ is —OCHBr₂. In embodiments, R³ is —OCHF₂. In embodiments, R³ is —OCHI₂. In embodiments, R³ is hydrogen or unsubstituted C₁-C₄ alkyl. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂J, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

In embodiments, R⁴ is independently hydrogen. In embodiments, R⁴ is independently halogen. In embodiments, R⁴ is independently —F. In embodiments, R⁴ is independently —Cl. In embodiments, R⁴ is independently —Br. In embodiments, R⁴ is independently —I. In embodiments, R⁴ is independently —CCl₃. In embodiments, R⁴ is independently —CBr₃. In embodiments, R⁴ is independently —CF₃. In embodiments, R⁴ is independently —CI₃. In embodiments, R⁴ is independently —CH₂C₁. In embodiments, R⁴ is independently —CH₂Br. In embodiments, R⁴ is independently —CH₂F. In embodiments, R⁴ is independently —CH₂I. In embodiments, R⁴ is independently —CHCl₂. In embodiments, R⁴ is independently —CHBr₂. In embodiments, R⁴ is independently —CHF₂. In embodiments, R⁴ is independently —CHI₂. In embodiments, R⁴ is independently —CN. In embodiments, R⁴ is independently —OH. In embodiments, R⁴ is independently —NH₂. In embodiments, R⁴ is independently —COOH. In embodiments, R⁴ is independently —CONH₂. In embodiments, R⁴ is independently —NO₂. In embodiments, R⁴ is independently —SH. In embodiments, R⁴ is independently —SO₃H. In embodiments, R⁴ is independently —OSO₃H. In embodiments, R⁴ is independently —SO₂NH₂. In embodiments, R⁴ is independently —NHNH₂. In embodiments, R⁴ is independently —ONH₂. In embodiments, R⁴ is independently —NHC(O)NHNH₂. In embodiments, R⁴ is independently —OCCl₃. In embodiments, R⁴ is independently —OCBr₃. In embodiments, R⁴ is independently —OCF₃. In embodiments, R⁴ is independently —OCI₃. In embodiments, R⁴ is independently —OCH₂C₁. In embodiments, R⁴ is independently —OCH₂Br. In embodiments, R⁴ is independently —OCH₂F. In embodiments, R⁴ is independently —OCH₂I. In embodiments, R⁴ is independently —OCHCl₂. In embodiments, R⁴ is independently —OCHBr₂. In embodiments, R⁴ is independently —OCHF₂. In embodiments, R⁴ is independently —OCHI₂. In embodiments, R⁴ is independently hydrogen or unsubstituted C₁-C₄ alkyl. In embodiments, R⁴ is independently unsubstituted C₁-C₄ alkyl. In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted n-propyl. In embodiments, R⁴ is independently unsubstituted isopropyl. In embodiments, R⁴ is independently unsubstituted butyl. In embodiments, R⁴ is independently unsubstituted n-butyl. In embodiments, R⁴ is independently unsubstituted isobutyl. In embodiments, R⁴ is independently unsubstituted tert-butyl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

In embodiments, R⁵ is hydrogen. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CCl₃. In embodiments, R⁵ is —CBr₃. In embodiments, R⁵ is —CF₃. In embodiments, R⁵ is —CI₃. In embodiments, R⁵ is —CH₂C₁. In embodiments, R⁵ is —CH₂Br. In embodiments, R⁵ is —CH₂F. In embodiments, R⁵ is —CH₂I. In embodiments, R⁵ is —CHCl₂. In embodiments, R⁵ is —CHBr₂. In embodiments, R⁵ is —CHF₂. In embodiments, R⁵ is —CHI₂. In embodiments, R⁵ is —CN. In embodiments, R⁵ is —OH. In embodiments, R⁵ is —NH₂. In embodiments, R⁵ is —COOH. In embodiments, R⁵ is —CONH₂. In embodiments, R⁵ is —NO₂. In embodiments, R⁵ is —SH. In embodiments, R⁵ is —SO₃H. In embodiments, R⁵ is —OSO₃H. In embodiments, R⁵ is —SO₂NH₂. In embodiments, R⁵ is —NHNH₂. In embodiments, R⁵ is —ONH₂. In embodiments, R⁵ is —NHC(O)NHNH₂. In embodiments, R⁵ is —OCCl₃. In embodiments, R⁵ is —OCBr₃. In embodiments, R⁵ is —OCF₃. In embodiments, R⁵ is —OCI₃. In embodiments, R⁵ is —OCH₂C₁. In embodiments, R⁵ is —OCH₂Br. In embodiments, R⁵ is —OCH₂F. In embodiments, R⁵ is —OCH₂I. In embodiments, R⁵ is —OCHCl₂. In embodiments, R⁵ is —OCHBr₂. In embodiments, R⁵ is —OCHF₂. In embodiments, R⁵ is —OCHI₂. In embodiments, R⁵ is hydrogen or unsubstituted C₁-C₄ alkyl. In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₈ alkyl, or substituted or unsubstituted 2 to 6 membered heteroalkyl.

In embodiments, R⁶ is hydrogen. In embodiments, R⁶ is halogen. In embodiments, R⁶ is —F. In embodiments, R⁶ is —Cl. In embodiments, R⁶ is —Br. In embodiments, R⁶ is —I. In embodiments, R⁶ is —CCl₃. In embodiments, R⁶ is —CBr₃. In embodiments, R⁶ is —CF₃. In embodiments, R⁶ is —CI₃. In embodiments, R⁶ is —CH₂C₁. In embodiments, R⁶ is —CH₂Br. In embodiments, R⁶ is —CH₂F. In embodiments, R⁶ is —CH₂I. In embodiments, R⁶ is —CHCl₂. In embodiments, R⁶ is —CHBr₂. In embodiments, R⁶ is —CHF₂. In embodiments, R⁶ is —CHI₂. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃H. In embodiments, R⁶ is —OSO₃H. In embodiments, R⁶ is —SO₂NH₂. In embodiments, R⁶ is —NHNH₂. In embodiments, R⁶ is —ONH₂. In embodiments, R⁶ is —NHC(O)NHNH₂. In embodiments, R⁶ is —OCCl₃. In embodiments, R⁶ is —OCBr₃. In embodiments, R⁶ is —OCF₃. In embodiments, R⁶ is —OCI₃. In embodiments, R⁶ is —OCH₂C₁. In embodiments, R⁶ is —OCH₂Br. In embodiments, R⁶ is —OCH₂F. In embodiments, R⁶ is —OCH₂I. In embodiments, R⁶ is —OCHCl₂. In embodiments, R⁶ is —OCHBr₂. In embodiments, R⁶ is —OCHF₂. In embodiments, R⁶ is —OCHI₂. In embodiments, R⁶ is hydrogen or unsubstituted C₁-C₄ alkyl. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl.

In embodiments, a substituted R¹⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁴ is substituted with a plurality of groups selected from substituent groups, size-limited substituent groups, and lower substituent groups; each substituent group, size-limited substituent group, and/or lower substituent group may optionally be different. In embodiments, when R¹⁴ is substituted, it is substituted with at least one substituent group. In embodiments, when R¹⁴ is substituted, it is substituted with at least one size-limited substituent group. In embodiments, when R¹⁴ is substituted, it is substituted with at least one lower substituent group.

In embodiments, R¹⁴ is hydrogen, hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted C₁-C₆ alkyl, or substituted or unsubstituted 2 to 6 membered heteroalkyl.

In embodiments, R¹⁴ is hydrogen. In embodiments, R¹⁴ is —CCl₃. In embodiments, R¹⁴ is —CBr₃. In embodiments, R¹⁴ is —CF₃. In embodiments, R¹⁴ is —CI₃. In embodiments, R¹⁴ is —CH₂C₁. In embodiments, R¹⁴ is —CH₂Br. In embodiments, R¹⁴ is —CH₂F. In embodiments, R¹⁴ is —CH₂I. In embodiments, R¹⁴ is —CHCl₂. In embodiments, R¹⁴ is —CHBr₂. In embodiments, R¹⁴ is —CHF₂. In embodiments, R¹⁴ is —CHI₂. In embodiments, R¹⁴ is —CN. In embodiments, R¹⁴ is —OH. In embodiments, R¹⁴ is —NH₂. In embodiments, R¹⁴ is —COOH. In embodiments, R¹⁴ is —CONH₂. In embodiments, R¹⁴ is —OCCl₃. In embodiments, R¹⁴ is —OCBr₃. In embodiments, R¹⁴ is —OCF₃. In embodiments, R¹⁴ is —OCI₃. In embodiments, R¹⁴ is —OCH₂C₁. In embodiments, R¹⁴ is —OCH₂Br. In embodiments, R¹⁴ is —OCH₂F. In embodiments, R¹⁴ is —OCH₂I. In embodiments, R¹⁴ is —OCHCl₂. In embodiments, R¹⁴ is —OCHBr₂. In embodiments, R¹⁴ is —OCHF₂. In embodiments, R¹⁴ is —OCHI₂. In embodiments, R¹⁴ is hydrogen or unsubstituted C₁-C₄ alkyl. In embodiments, R¹⁴ is unsubstituted C₁-C₄ alkyl. In embodiments, R¹⁴ is unsubstituted methyl. In embodiments, R¹⁴ is unsubstituted ethyl. In embodiments, R¹⁴ is unsubstituted propyl. In embodiments, R¹⁴ is unsubstituted n-propyl. In embodiments, R¹⁴ is unsubstituted isopropyl. In embodiments, R¹⁴ is unsubstituted butyl. In embodiments, R¹⁴ is unsubstituted n-butyl. In embodiments, R¹⁴ is unsubstituted isobutyl. In embodiments, R¹⁴ is unsubstituted tert-butyl.

In embodiments of Formula (Ia) or Formula (I), R′ is hydrogen.

In embodiments of Formula (Ia) or Formula (I), R² is hydrogen or unsubstituted C₁-C₄ alkyl. In embodiments of Formula (Ia) or Formula (I), R² is unsubstituted methyl, unsubstituted ethyl, or unsubstituted propyl.

In embodiments of Formula (Ia) or Formula (I), R³, R⁴, R⁵, and R⁶ are hydrogen.

In embodiments of Formula (IIa) or Formula (II), R¹ is —CF₃ or substituted or unsubstituted C₁-C₄ alkyl. In embodiments of Formula (IIa) or Formula (II), R¹ is —CF₃ or substituted or unsubstituted methyl.

In embodiments of Formula (IIa) or Formula (II), R², R³, R⁴, R⁵, and R⁶ are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl. In embodiments of Formula (IIa) or Formula (II), R², R³, R⁴, R⁵, and R⁶ are hydrogen.

In embodiments, R¹ and R² are each independently substituted or unsubstituted alkyl.

In embodiments, R¹ and R² are each independently substituted or unsubstituted C₁0.6 alkyl.

In embodiments, R¹ and R² are each independently unsubstituted C₁₋₆ alkyl.

In embodiments, R¹ and R² are each independently substituted C₁₋₆ alkyl wherein the substituent is one or more halogen.

In embodiments, at least one of R¹, R², R³, R⁴, R⁵, R⁶, and R⁷ is a substituent other than hydrogen.

In embodiments, R³, R⁴, R⁵, R⁶, and R⁷ are hydrogen.

In embodiments, R¹, R³, R⁴, R⁵, R⁶, and R⁷ are hydrogen, and R² is a substituent other than hydrogen.

In embodiments, R², R³, R⁴, R⁵, R⁶, and R⁷ are hydrogen, and R² is a substituent other than hydrogen.

In embodiments, R¹ is —CH₃, and R², R³, R⁴, R⁵, R⁶, and R⁷ are hydrogen.

In embodiments, R′ is —CF₃, and R², R³, R⁴, R⁵, R⁶, and R⁷ are hydrogen.

In embodiments, R² is —CH₃, and R¹, R³, R⁴, R⁵, R⁶, and R⁷ are hydrogen.

In embodiments, R² is —CH₂CH₃, and R¹, R³, R⁴, R⁵, R⁶, and R⁷ are hydrogen.

In embodiments, WSG is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₅, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted WSG (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted WSG is substituted with a plurality of groups selected from substituent groups, size-limited substituent groups, and lower substituent groups; each substituent group, size-limited substituent group, and/or lower substituent group may optionally be different. In embodiments, when WSG is substituted, it is substituted with at least one substituent group. In embodiments, when WSG is substituted, it is substituted with at least one size-limited substituent group. In embodiments, when WSG is substituted, it is substituted with at least one lower substituent group.

In embodiments, WSG is hydrogen, R³³-substituted or unsubstituted C₁-C₁₀ alkyl, R³³-substituted or unsubstituted 2 to 10 membered heteroalkyl, R³³-substituted or unsubstituted C₃-C₁₀ cycloalkyl, R³³-substituted or unsubstituted 3 to 10 membered heterocycloalkyl, R³³-substituted or unsubstituted C₆-C₁₀ aryl, or R³³-substituted or unsubstituted 5 to 10 membered heteroaryl.

R³³ is independently halogen, —OR³⁴, —NR³⁵R³⁶, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered).

R³⁴, R³, and R³⁶ are independently hydrogen, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered).

R³⁷ is independently halogen, —OR³⁸, —NR³⁹R⁴⁰, R⁴¹-substituted or unsubstituted alkyl (e.g., C₁-C₈, C₁-C₆, C₁-C₄, or C₁-C₂), R^{4′}-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), —(R⁴¹-substituted or unsubstituted alkylene)-(R⁴¹-substituted or unsubstituted heterocycloalkyl), R⁴¹-substituted or unsubstituted aryl (e.g., C₆-C₁₀ or phenyl), or R⁴¹-substituted or unsubstituted heteroaryl (e.g., 5 to 10 membered, 5 to 9 membered, or 5 to 6 membered).

R⁴¹ is independently halogen, —OR³, —NR³⁹R⁴⁰, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³⁸, R³⁹, and R⁴⁰ are independently hydrogen, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

WSG is a water soluble group. In embodiments, the WSG group in Formula I serves to alter the solubility of the compounds of Formula I in an aqueous system. In embodiments, WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁ heteroarylene, wherein the alkyl, heteroalkyl, cycloalkyl, heterocycloalkyl, arylene, or heteroarylene is optionally substituted with one or more R³³; wherein each R³³ is independently halogen, —OR³⁴, —NR³⁵R³⁶, C₁-C₁₀ alkyl, C₁-C₁₀ heteroalkyl, C₁-C₁ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R³⁷; each R³⁴, R³⁵ and R³⁶ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R³⁷; each R³⁷ is independently halogen, —OR³⁸, —NR³⁹R⁴⁰, C₁-C₁₀ alkyl, C₁-C₁₀ heteroalkyl, C₁-C₁₀ cycloalkyl, C₁-C₁₀ heterocycloalkyl, —(C₁-C₆alkyl)(C₁-C₁₀ heterocycloalkyl), C₁-C₁₀ arylene, or C₁-C₁₀ heteroarylene; and each of R³⁸, R³⁹ and R⁴⁰ is independently hydrogen or C₁-C₁₀ alkyl.

WSG is a water soluble group. The WSG group in Formula I serves to alter the solubility of the compounds of Formula I in an aqueous system. In embodiments, WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R³³; wherein each R³³ is independently halogen, —OR³⁴, —NR³⁵R³⁶, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R³⁷; each R³⁴, R³⁵ and R³⁶ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R³⁷; each R³⁷ is independently halogen, —OR³⁸, —NR³⁹R⁴⁰, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and each of R³⁸, R³⁹ and R⁴⁰ is independently hydrogen or C₁-C₁₀ alkyl.

In embodiments, R³³ is independently halogen, —OR³⁴, —NR³⁵R³⁶, R³⁷-substituted or unsubstituted C₁-C₁₀ alkyl, R³⁷-substituted or unsubstituted 2 to 10 membered heteroalkyl, R³⁷-substituted or unsubstituted C₃-C₁₀ cycloalkyl, R³⁷-substituted or unsubstituted 3 to 10 membered heterocycloalkyl, R³⁷-substituted or unsubstituted C₆-C₁₀ aryl, or R³⁷-substituted or unsubstituted 5 to 10 membered heteroaryl.

In embodiments, R³³ is independently halogen. In embodiments, R³³ is independently —F. In embodiments, R³³ is independently —Cl. In embodiments, R³³ is independently —Br. In embodiments, R³³ is independently —I. In embodiments, R³³ is independently —OR³⁴. In embodiments, R³³ is independently —OH. In embodiments, R³³ is independently —NR³⁵R³⁶. In embodiments, R³³ is independently —NH₂. In embodiments, R³³ is independently R³⁷-substituted or unsubstituted C₁-C₁₀ alkyl. In embodiments, R³³ is independently unsubstituted C₁-C₄ alkyl. In embodiments, R³³ is independently unsubstituted methyl. In embodiments, R³³ is independently unsubstituted ethyl. In embodiments, R³³ is independently unsubstituted propyl. In embodiments, R³³ is independently unsubstituted n-propyl. In embodiments, R³³ is independently unsubstituted isopropyl. In embodiments, R³³ is independently unsubstituted butyl. In embodiments, R³³ is independently unsubstituted n-butyl. In embodiments, R³³ is independently unsubstituted tert-butyl. In embodiments, R³³ is independently R³⁷-substituted or unsubstituted 2 to 10 membered heteroalkyl. In embodiments, R³³ is independently unsubstituted 2 to 4 membered heteroalkyl. In embodiments, R³³ is independently R³⁷-substituted or unsubstituted C₃-C₁₀ cycloalkyl. In embodiments, R³³ is independently R³⁷-substituted or unsubstituted 3 to 10 membered heterocycloalkyl. In embodiments, R³³ is independently R³⁷-substituted or unsubstituted C₆-C₁₀ aryl. In embodiments, R³³ is independently unsubstituted phenyl. In embodiments, R³³ is independently R³⁷-substituted or unsubstituted 5 to 10 membered heteroaryl. In embodiments, R³³ is independently unsubstituted triazole. In embodiments, R³³ is independently unsubstituted 1,2,4-triazole. In embodiments, R³³ is independently unsubstituted 1,2,3-triazole. In embodiments, R³³ is independently unsubstituted imidazole. In embodiments, R³³ is independently unsubstituted pyrazole.

In embodiments, R³⁴ is independently hydrogen. In embodiments, R³⁴ is independently R³⁷-substituted or unsubstituted C₁-C₁₀ alkyl. In embodiments, R³⁴ is independently unsubstituted C₁-C₄ alkyl. In embodiments, R³⁴ is independently unsubstituted methyl. In embodiments, R³⁴ is independently unsubstituted ethyl. In embodiments, R³⁴ is independently unsubstituted propyl. In embodiments, R³⁴ is independently unsubstituted n-propyl. In embodiments, R³⁴ is independently unsubstituted isopropyl. In embodiments, R³⁴ is independently unsubstituted butyl. In embodiments, R³⁴ is independently unsubstituted n-butyl. In embodiments, R³⁴ is independently unsubstituted tert-butyl. In embodiments, R³⁴ is independently R³⁷-substituted or unsubstituted 2 to 10 membered heteroalkyl. In embodiments, R³⁴ is independently unsubstituted 2 to 4 membered heteroalkyl. In embodiments, R³⁴ is independently R³⁷-substituted or unsubstituted C₃-C₁₀ cycloalkyl. In embodiments, R³⁴ is independently R³⁷-substituted or unsubstituted 3 to 10 membered heterocycloalkyl. In embodiments, R³⁴ is independently R³⁷-substituted or unsubstituted C₆-C₁₀ aryl. In embodiments, R³⁴ is independently unsubstituted phenyl. In embodiments, R³⁴ is independently R³⁷-substituted or unsubstituted 5 to 10 membered heteroaryl.

In embodiments, R³⁵ is independently hydrogen. In embodiments, R³⁵ is independently R³⁷-substituted or unsubstituted C₁-C₁₀ alkyl. In embodiments, R³⁵ is independently unsubstituted C₁-C₄ alkyl. In embodiments, R³⁵ is independently unsubstituted methyl. In embodiments, R³⁵ is independently unsubstituted ethyl. In embodiments, R³⁵ is independently unsubstituted propyl. In embodiments, R³⁵ is independently unsubstituted n-propyl. In embodiments, R³⁵ is independently unsubstituted isopropyl. In embodiments, R³⁵ is independently unsubstituted butyl. In embodiments, R³⁵ is independently unsubstituted n-butyl. In embodiments, R³⁵ is independently unsubstituted tert-butyl. In embodiments, R³⁵ is independently R³⁷-substituted or unsubstituted 2 to 10 membered heteroalkyl. In embodiments, R³⁵ is independently unsubstituted 2 to 4 membered heteroalkyl. In embodiments, R³⁵ is independently R³⁷-substituted or unsubstituted C₃-C₁₀ cycloalkyl. In embodiments, R³⁵ is independently R³⁷-substituted or unsubstituted 3 to 10 membered heterocycloalkyl. In embodiments, R³⁵ is independently R³⁷-substituted or unsubstituted C₆-C₁₀ aryl. In embodiments, R³⁵ is independently unsubstituted phenyl. In embodiments, R³⁵ is independently R³⁷-substituted or unsubstituted 5 to 10 membered heteroaryl.

In embodiments, R³⁶ is independently hydrogen. In embodiments, R³⁶ is independently R³⁷-substituted or unsubstituted C₁-C₁₀ alkyl. In embodiments, R³⁶ is independently unsubstituted C₁-C₄ alkyl. In embodiments, R³⁶ is independently unsubstituted methyl. In embodiments, R³⁶ is independently unsubstituted ethyl. In embodiments, R³⁶ is independently unsubstituted propyl. In embodiments, R³⁶ is independently unsubstituted n-propyl. In embodiments, R³⁶ is independently unsubstituted isopropyl. In embodiments, R³⁶ is independently unsubstituted butyl. In embodiments, R³⁶ is independently unsubstituted n-butyl. In embodiments, R³⁶ is independently unsubstituted tert-butyl. In embodiments, R³⁶ is independently R³⁷-substituted or unsubstituted 2 to 10 membered heteroalkyl. In embodiments, R³⁶ is independently unsubstituted 2 to 4 membered heteroalkyl. In embodiments, R³⁶ is independently R³⁷-substituted or unsubstituted C₃-C₁₀ cycloalkyl. In embodiments, R³⁶ is independently R³⁷-substituted or unsubstituted 3 to 10 membered heterocycloalkyl. In embodiments, R³⁶ is independently R³⁷-substituted or unsubstituted C₆-C₁₀ aryl. In embodiments, R³⁶ is independently unsubstituted phenyl. In embodiments, R³⁶ is independently R³⁷-substituted or unsubstituted 5 to 10 membered heteroaryl.

In embodiments, R³⁴, R³⁵, and R³⁶ are independently hydrogen, R³⁷-substituted or unsubstituted C₁-C₁₀ alkyl, R³⁷-substituted or unsubstituted 2 to 10 membered heteroalkyl, R³⁷-substituted or unsubstituted C₃-C₁₀ cycloalkyl, R³⁷-substituted or unsubstituted 3 to 10 membered heterocycloalkyl, R³⁷-substituted or unsubstituted C₆-C₁₀ aryl, or R³⁷-substituted or unsubstituted 5 to 10 membered heteroaryl.

In embodiments, R³⁷ is independently halogen, —OR³⁸, —NR³⁹R⁴⁰, R⁴¹-substituted or unsubstituted C₁-C₁₀ alkyl, R⁴¹-substituted or unsubstituted 2 to 10 membered heteroalkyl, R⁴¹-substituted or unsubstituted C₃-C₁₀ cycloalkyl, R⁴¹-substituted or unsubstituted 3 to 10 membered heterocycloalkyl, —(R⁴¹-substituted or unsubstituted C₁-C₆ alkylene)-(R⁴¹-substituted or unsubstituted 3 to 10 membered heterocycloalkyl), R⁴¹-substituted or unsubstituted C₆-C₁₀ aryl, or R⁴¹-substituted or unsubstituted 5 to 10 membered heteroaryl.

In embodiments, R³⁷ is independently halogen. In embodiments, R³⁷ is independently —F. In embodiments, R³⁷ is independently —Cl. In embodiments, R³⁷ is independently —Br. In embodiments, R³⁷ is independently —I. In embodiments, R³⁷ is independently —OR³⁸. In embodiments, R³⁷ is independently —OH. In embodiments, R³⁷ is independently —NR³⁹R⁴⁰. In embodiments, R³⁷ is independently —NH₂. In embodiments, R³⁷ is independently unsubstituted C₁-C₁₀ alkyl. In embodiments, R³⁷ is independently unsubstituted C₁-C₄ alkyl. In embodiments, R³⁷ is independently unsubstituted methyl. In embodiments, R³⁷ is independently unsubstituted ethyl. In embodiments, R³⁷ is independently unsubstituted propyl. In embodiments, R³⁷ is independently unsubstituted n-propyl. In embodiments, R³⁷ is independently unsubstituted isopropyl. In embodiments, R³⁷ is independently unsubstituted butyl. In embodiments, R³⁷ is independently unsubstituted n-butyl. In embodiments, R³⁷ is independently unsubstituted tert-butyl. In embodiments, R³⁷ is independently unsubstituted 2 to 10 membered heteroalkyl. In embodiments, R³⁷ is independently unsubstituted 2 to 4 membered heteroalkyl. In embodiments, R³⁷ is independently unsubstituted C₃-C₁₀ cycloalkyl. In embodiments, R³⁷ is independently unsubstituted 3 to 10 membered heterocycloalkyl. In embodiments, R³⁷ is independently -(unsubstituted C₁-C₆ alkylene)-(R⁴¹-substituted or unsubstituted 3 to 10 membered heterocycloalkyl). In embodiments, R³⁷ is independently -(unsubstituted methylene)-(R⁴¹-substituted or unsubstituted 3 to 10 membered heterocycloalkyl). In embodiments, R³⁷ is independently -(unsubstituted ethylene)-(R⁴¹-substituted or unsubstituted 3 to 10 membered heterocycloalkyl). In embodiments, R³⁷ is independently -(unsubstituted propylene)-(R⁴¹-substituted or unsubstituted 3 to 10 membered heterocycloalkyl). In embodiments, R³⁷ is independently -(unsubstituted butylene)-(R⁴¹-substituted or unsubstituted 3 to 10 membered heterocycloalkyl). In embodiments, R³⁷ is independently -(unsubstituted pentylene)-(R⁴¹-substituted or unsubstituted 3 to 10 membered heterocycloalkyl). In embodiments, R³⁷ is independently -(unsubstituted hexylene)-(R⁴¹-substituted or unsubstituted 3 to 10 membered heterocycloalkyl). In embodiments, R³⁷ is independently unsubstituted C₆-C₁₀ aryl. In embodiments, R³⁷ is independently unsubstituted phenyl. In embodiments, R³⁷ is independently unsubstituted 5 to 10 membered heteroaryl.

In embodiments, R³⁸ is independently hydrogen. In embodiments, R³⁸ is independently unsubstituted C₁-C₄ alkyl. In embodiments, R³⁸ is independently unsubstituted methyl. In embodiments, R³⁸ is independently unsubstituted ethyl. In embodiments, R³⁸ is independently unsubstituted propyl. In embodiments, R³⁸ is independently unsubstituted n-propyl. In embodiments, R³⁸ is independently unsubstituted isopropyl. In embodiments, R³⁸ is independently unsubstituted butyl. In embodiments, R³⁸ is independently unsubstituted n-butyl. In embodiments, R³⁸ is independently unsubstituted tert-butyl. In embodiments, R³⁸ is independently unsubstituted 2 to 10 membered heteroalkyl. In embodiments, R³⁸ is independently unsubstituted C₃-C₁₀ cycloalkyl. In embodiments, R³⁸ is independently unsubstituted 3 to 10 membered heterocycloalkyl. In embodiments, R³⁸ is independently unsubstituted C₆-C₁₀ aryl. In embodiments, R³⁸ is independently unsubstituted 5 to 10 membered heteroaryl.

In embodiments, R³⁹ is independently hydrogen. In embodiments, R³⁹ is independently unsubstituted C₁-C₄ alkyl. In embodiments, R³⁹ is independently unsubstituted methyl. In embodiments, R³⁹ is independently unsubstituted ethyl. In embodiments, R³⁹ is independently unsubstituted propyl. In embodiments, R³⁹ is independently unsubstituted n-propyl. In embodiments, R³⁹ is independently unsubstituted isopropyl. In embodiments, R³⁹ is independently unsubstituted butyl. In embodiments, R³⁹ is independently unsubstituted n-butyl. In embodiments, R³⁹ is independently unsubstituted tert-butyl. In embodiments, R³⁹ is independently unsubstituted 2 to 10 membered heteroalkyl. In embodiments, R³⁹ is independently unsubstituted C₃-C₁₀ cycloalkyl. In embodiments, R³⁹ is independently unsubstituted 3 to 10 membered heterocycloalkyl. In embodiments, R³⁹ is independently unsubstituted C₆-C₁₀ aryl. In embodiments, R³⁹ is independently unsubstituted 5 to 10 membered heteroaryl.

In embodiments, R⁴⁰ is independently hydrogen. In embodiments, R⁴⁰ is independently unsubstituted C₁-C₄ alkyl. In embodiments, R⁴⁰ is independently unsubstituted methyl. In embodiments, R⁴⁰ is independently unsubstituted ethyl. In embodiments, R⁴⁰ is independently unsubstituted propyl. In embodiments, R⁴⁰ is independently unsubstituted n-propyl. In embodiments, R⁴⁰ is independently unsubstituted isopropyl. In embodiments, R⁴⁰ is independently unsubstituted butyl. In embodiments, R⁴⁰ is independently unsubstituted n-butyl. In embodiments, R⁴⁰ is independently unsubstituted tert-butyl. In embodiments, R⁴⁰ is independently unsubstituted 2 to 10 membered heteroalkyl. In embodiments, R⁴⁰ is independently unsubstituted C₃-C₁₀ cycloalkyl. In embodiments, R⁴⁰ is independently unsubstituted 3 to 10 membered heterocycloalkyl. In embodiments, R⁴⁰ is independently unsubstituted C₆-C₁₀ aryl. In embodiments, R⁴⁰ is independently unsubstituted 5 to 10 membered heteroaryl.

In embodiments, R³⁸, R³⁹, and R⁴⁰ are independently hydrogen, unsubstituted C₁-C₁₀ alkyl, unsubstituted 2 to 10 membered heteroalkyl, unsubstituted C₃-C₁₀ cycloalkyl, unsubstituted 3 to 10 membered heterocycloalkyl, unsubstituted C₆-C₁₀ aryl, or unsubstituted 5 to 10 membered heteroaryl. In embodiments, R³⁸, R³⁹, and R⁴⁰ are independently hydrogen or unsubstituted C₁-C₁₀ alkyl.

In embodiments, R⁴¹ is independently halogen, —OR³⁸, —NR³⁹R⁴⁰, unsubstituted C₁-C₁₀ alkyl, unsubstituted 2 to 10 membered heteroalkyl, unsubstituted C₃-C₁₀ cycloalkyl, unsubstituted 3 to 10 membered heterocycloalkyl, unsubstituted C₆-C₁₀ aryl, or unsubstituted 5 to 10 membered heteroaryl.

In embodiments, R⁴¹ is independently halogen. In embodiments, R⁴¹ is independently —F. In embodiments, R⁴¹ is independently —Cl. In embodiments, R⁴¹ is independently —Br. In embodiments, R⁴¹ is independently —I. In embodiments, R⁴¹ is independently —OR³⁸. In embodiments, R^{4.1} is independently —OH. In embodiments, R⁴¹ is independently —NR³⁹R⁴⁰. In embodiments, R⁴¹ is independently —NH₂. In embodiments, R⁴¹ is independently unsubstituted C₁-C₁₀ alkyl. In embodiments, R⁴¹ is independently unsubstituted C₁-C₄ alkyl. In embodiments, R⁴¹ is independently unsubstituted methyl. In embodiments, R⁴¹ is independently unsubstituted ethyl. In embodiments, R⁴¹ is independently unsubstituted propyl. In embodiments, R⁴¹ is independently unsubstituted n-propyl. In embodiments, R⁴¹ is independently unsubstituted isopropyl. In embodiments, R⁴¹ is independently unsubstituted butyl. In embodiments, R⁴¹ is independently unsubstituted n-butyl. In embodiments, R⁴¹ is independently unsubstituted tert-butyl. In embodiments, R⁴¹ is independently unsubstituted 2 to 10 membered heteroalkyl. In embodiments, R⁴¹ is independently unsubstituted 2 to 4 membered heteroalkyl. In embodiments, R⁴¹ is independently unsubstituted C₃-C₁₀ cycloalkyl. In embodiments, R⁴¹ is independently unsubstituted 3 to 10 membered heterocycloalkyl.

In embodiments, the WSG is

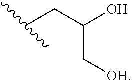 [SMILES: *CC(O)CO]

In embodiments, WSG is polyethylene glycol, polypropylene glycol, co-polymer of polyethylene glycol and polypropylene glycol, or alkoxy derivatives thereof. In embodiments, WSG is

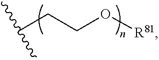

wherein n is an integer from 1 to 50 and R⁸¹ is hydrogen or substituted or unsubstituted alkyl (e.g., C₁-C₈, C₁-C₆, C₁-C₄, or C₁-C₂).

In embodiments, a substituted R⁸¹ (e.g., substituted alkyl) is substituted with at least one substituent group, size-limited substituent group, or lower substituent group; wherein if the substituted R⁸¹ is substituted with a plurality of groups selected from substituent groups, size-limited substituent groups, and lower substituent groups; each substituent group, size-limited substituent group, and/or lower substituent group may optionally be different. In embodiments, when R⁸¹ is substituted, it is substituted with at least one substituent group. In embodiments, when R⁸¹ is substituted, it is substituted with at least one size-limited substituent group. In embodiments, when R⁸¹ is substituted, it is substituted with at least one lower substituent group.

In embodiments, R⁸¹ is hydrogen, substituted or unsubstituted alkyl (e.g., C₁-C₈, C₁-C₆, C₁-C₄, or C₁-C₂), substituted or unsubstituted alkenyl (e.g., C₂-C₅, C₂-C₆, or C₂-C₄), or substituted or unsubstituted alkynyl (e.g., C₂-C₈, C₂-C₆, or C₂-C₄). In embodiments, R¹ is hydrogen, substituted or unsubstituted C₁-C₁₀ alkyl, substituted or unsubstituted C₂-C₁₀alkenyl, or substituted or unsubstituted C₂-C₁₀ alkynyl. In embodiments, R⁸¹ is hydrogen. In embodiments, R⁸¹ is unsubstituted C₁-C₄ alkyl. In embodiments, R¹ is unsubstituted methyl. In embodiments, R⁸¹ is unsubstituted ethyl. In embodiments, R⁸¹ is unsubstituted propyl. In embodiments, R⁸¹ is unsubstituted n-propyl. In embodiments, R⁸¹ is unsubstituted isopropyl. In embodiments, R⁸¹ is unsubstituted butyl. In embodiments, R⁸¹ is unsubstituted n-butyl. In embodiments, R⁸¹ is unsubstituted tert-butyl. In embodiments, R⁸¹ is —CH₂—C≡CH.

In embodiments, WSG is polyethylene glycol, polypropylene glycol, co-polymer of polyethylene glycol and polypropylene glycol, or alkoxy derivatives thereof. In embodiments, WSG is

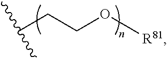

wherein n is an integer from 1 to 50 and R⁸¹ is hydrogen, C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In embodiments, WSG is

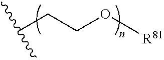

and R⁸¹ is hydrogen. In embodiments, WSG is

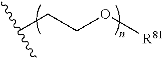

and R⁸¹ is methyl. In embodiments, WSG is

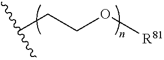

and R⁸¹ is ethyl.

In embodiments, WSG is

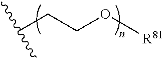

and R⁸¹ is —CH₂—C≡CH.

In embodiments, WSG is

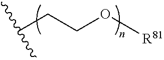

and n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In embodiments, WSG is

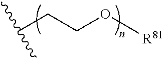

and n is an integer of value 1-10, 1-20, 1-30, 1-40, 1-50, 10-20, 10-30, 10-40, 10-50, 20-30, 20-40, 20-50, 30-40, 30-50, or 40-50.

In embodiments, WSG is

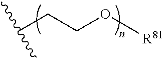

and n is 1, 2, 3, 4, 5, 6, 7, 8, 9, or 10. In embodiments, WSG is

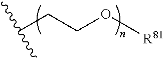

and n is 3 or 6.

In embodiments, WSG is

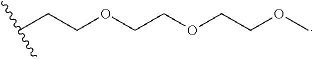 [SMILES: *CCOCCOCCOC ; "."]

In embodiments, WSG is

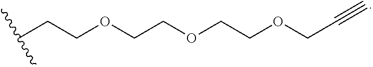 [SMILES: *CCOCCOCCOCC#C ; "."]

In embodiments, the WSG is

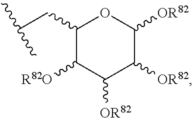

wherein each R⁸² is hydrogen or substituted or unsubstituted alkyl (e.g., C₁-C₈, C₁-C₆, C₁-C₄, or C₁-C₂).

In embodiments, a substituted R⁸² (e.g., substituted alkyl) is substituted with at least one substituent group, size-limited substituent group, or lower substituent group; wherein if the substituted R⁸² is substituted with a plurality of groups selected from substituent groups, size-limited substituent groups, and lower substituent groups; each substituent group, size-limited substituent group, and/or lower substituent group may optionally be different. In embodiments, when R⁸² is substituted, it is substituted with at least one substituent group. In embodiments, when R⁸² is substituted, it is substituted with at least one size-limited substituent group. In embodiments, when R⁸² is substituted, it is substituted with at least one lower substituent group.

In embodiments, R⁸² is independently hydrogen. In embodiments, R⁸² is independently unsubstituted C₁-C₁₀ alkyl. In embodiments, R⁸² is independently unsubstituted C₁-C₄ alkyl. In embodiments, R⁸² is independently unsubstituted methyl. In embodiments, R⁸² is independently unsubstituted ethyl. In embodiments, R⁸² is independently unsubstituted propyl. In embodiments, R⁸² is independently unsubstituted n-propyl. In embodiments, R⁸² is independently unsubstituted isopropyl. In embodiments, R⁸² is independently unsubstituted butyl. In embodiments, R⁸² is independently unsubstituted n-butyl. In embodiments, R⁸² is independently unsubstituted tert-butyl.

In embodiments, the WSG is

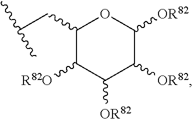

wherein each R⁸² is hydrogen or C₁-C₁₀ alkyl.

In embodiments, each R⁸² is independently a hydrogen, methyl, ethyl, propyl, or butyl.

In embodiments, the WSG is

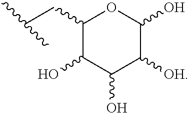 [SMILES: *CC1OC(O)C(O)C(O)C1O]

In embodiments, the WSG is

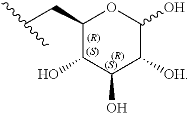

In embodiments, the WSG is

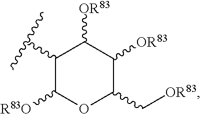

wherein each R⁸³ is hydrogen or substituted or unsubstituted alkyl (e.g., C₁-C₈, C₁-C₆, C₁-C₄, or C₁-C₂).

In embodiments, a substituted R⁸³ (e.g., substituted alkyl) is substituted with at least one substituent group, size-limited substituent group, or lower substituent group; wherein if the substituted R⁸³ is substituted with a plurality of groups selected from substituent groups, size-limited substituent groups, and lower substituent groups; each substituent group, size-limited substituent group, and/or lower substituent group may optionally be different. In embodiments, when R⁸³ is substituted, it is substituted with at least one substituent group. In embodiments, when R⁸³ is substituted, it is substituted with at least one size-limited substituent group. In embodiments, when R⁸³ is substituted, it is substituted with at least one lower substituent group.

In embodiments, R⁸³ is independently hydrogen. In embodiments, R⁸³ is independently unsubstituted C₁-C₁₀ alkyl. In embodiments, R⁸³ is independently unsubstituted C₁-C₄ alkyl. In embodiments, R⁸³ is independently unsubstituted methyl. In embodiments, R⁸³ is independently unsubstituted ethyl. In embodiments, R⁸³ is independently unsubstituted propyl. In embodiments, R⁸³ is independently unsubstituted n-propyl. In embodiments, R⁸³ is independently unsubstituted isopropyl. In embodiments, R⁸³ is independently unsubstituted butyl. In embodiments, R⁸³ is independently unsubstituted n-butyl. In embodiments, R⁸³ is independently unsubstituted tert-butyl.

In embodiments, the WSG is

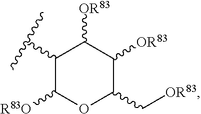

wherein each R⁸³ is hydrogen or C₁-C₁₀ alkyl. In embodiments, each R⁸³ is independently a hydrogen, methyl, ethyl, propyl, or butyl.

In embodiments, the WSG is

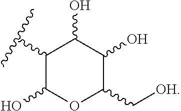

In some compounds of Formula I the WSG is

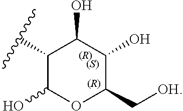

In embodiments, WSG is -(unsubstituted C₁-C₁₀ alkyl)-R³³—R³⁷. In embodiments, WSG is -(unsubstituted C₁-C₁₀ alkyl)-R³³—R³⁷, and R³³ is unsubstituted C₅-C₁₀ heteroarylene. In embodiments, WSG is -(unsubstituted C₁-C₁₀ alkyl)-R³³—R³⁷, and R³³ is unsubstituted C₅-C₁₀ heteroarylene and R³⁷ is -(unsubstituted C₁-C₆ alkyl)-(unsubstituted 3 to 10 membered heterocycloalkyl).

In embodiments, WSG is —(C₁-C₁₁ alkyl)-R³³—R³⁷. In embodiments, WSG is —(C₁-C₁₀ alkyl)-R³³—R³⁷ and R³³ is C₁-C₁₀ heteroarylene. In embodiments, WSG is —(C₁-C₁₀ alkyl)-R³³—R³⁷, R³³ is C₁-C₁₀ heteroarylene and R³⁷ is —(C₁-C₆alkyl)(C₁-C₁₀ heterocycloalkyl).

In embodiments, WSG is —CH₂—R³³—R³⁷.

In embodiments, WSG is —CH₂—R³³—R³⁷ and R³³ is unsubstituted triazolylene, unsubstituted imidazolylene, or unsubstituted pyrazolylene. In embodiments, WSG is —CH₂—R³³—R³⁷ and R³³ is unsubstituted triazolylene. In embodiments, WSG is —CH₂—R³³—R³⁷ and R³³ is unsubstituted 1,2,4-triazolylene. In embodiments, WSG is —CH₂—R³³—R³⁷ and R³³ is unsubstituted 1,2,3-triazolylene.

In embodiments, WSG is —CH₂—R³³—R³⁷ and R³³ is triazole, imidazole, or pyrazole.

In embodiments, WSG is —CH₂—R³³—R³⁷ and R³³ is triazole.

In embodiments, WSG is —CH₂—R³³—R³⁷ and R³³ is 1,2,4-triazole.

In embodiments, WSG is —CH₂—R³³—R³⁷ and R³³ is 1,2,3-triazole.

In embodiments, WSG is —CH₂—R³³—R³⁷, R³³ is unsubstituted 1,2,3-triazolylene and R³⁷ is -(unsubstituted C₁-C₆ alkylene)-(R⁴¹-substituted or unsubstituted 3 to 10 membered heterocycloalkyl). In embodiments, WSG is —CH₂—R³³—R³⁷, R³³ is 1,2,3-triazolylene and R³⁷ is —CH₂—(R⁴¹-substituted or unsubstituted 3 to 10 membered heterocycloalkyl). In embodiments, WSG is —CH₂—R³³—R³⁷, R³³ is 1,2,3-triazolylene and R³⁷ is —CH₂—(R⁴¹-substituted or unsubstituted tetrahydropyranyl). In embodiments, WSG is —CH₃—R³³—R³⁷, R³³ is unsubstituted 1,2,3-triazolylene, and R³⁷ is

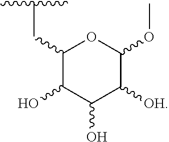 [SMILES: *CC1OC(OC)C(O)C(O)C1O]

In embodiments, WSG is —CH₂—R³³—R³⁷, R³³ is 1,2,3-triazole and R³⁷ is —(C₁-C₆alkyl)(C₁-C₁₀ heterocycloalkyl).

In embodiments, WSG is —CH₂—R³³—R³⁷, R³³ is 1,2,3-triazole and R³⁷ is —(C₁alkyl)(C₁-C₁₀ heterocycloalkyl).

In embodiments, WSG is —CH₂—R³³—R³⁷, R³³ is 1,2,3-triazole, R³⁷ is —(C₁alkyl)(C₁-C₁₀heretocycloalkyl), and C₁-C₁₀heretocycloalkyl is a tetrahydropyran derivative.

In embodiments, WSG is —CH₃—R³³—R³⁷, R³³ is 1,2,3-triazole, and R³⁷ is

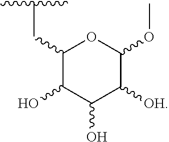 [SMILES: *CC1OC(OC)C(O)C(O)C1O]

In embodiments, WSG is

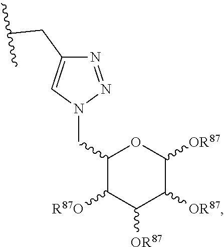

wherein each R⁸⁷ is independently hydrogen, unsubstituted alkyl (e.g., C₁-C₈, C₁-C₆, C₁-C₄, or C₁-C₂), or —C(═O)-(unsubstituted alkyl). In embodiments, WSG is

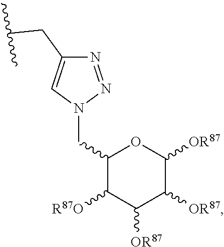

wherein each R⁸⁷ is independently hydrogen, unsubstituted C₁-C₁₀ alkyl, or —C(═O)-(unsubstituted C₁-C₁₀ alkyl). In embodiments, R⁸⁷ is independently hydrogen. In embodiments, R⁸⁷ is independently unsubstituted methyl. In embodiments, R⁸⁷ is independently unsubstituted ethyl. In embodiments, R⁸⁷ is independently unsubstituted propyl. In embodiments, R⁸⁷ is independently unsubstituted butyl. In embodiments, R⁸⁷ is independently unsubstituted acetyl. In embodiments, R⁸⁷ is independently unsubstituted propionyl. In embodiments, R⁸⁷ is independently unsubstituted butyryl. In embodiments, R⁸⁷ is independently hydrogen or methyl. In embodiments, R⁸⁷ is independently methyl or acetyl.

In embodiments, WSG is

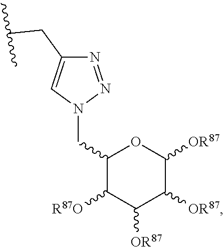

wherein each R⁸⁷ is hydrogen, C₁-C₁₀ alkyl, or —C(═O)C₁-C₁₀ alkyl. In embodiments, each R⁸⁷ is independently a hydrogen, methyl, ethyl, propyl, butyl, acetate, propionate, or butyrate. In embodiments, each R⁸⁷ is independently a hydrogen or methyl. In embodiments, each R⁸⁷ is independently a methyl or acetate.

In embodiments, WSG is

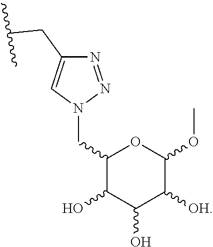 [SMILES: *Cc1cn(CC2OC(OC)C(O)C(O)C2O)nn1]

In embodiments, WSG is

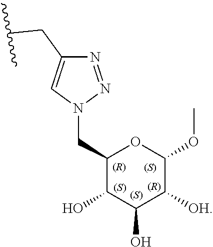

In embodiments, WSG is -(unsubstituted 2 to 10 membered heteroalkylene)-R³³-R³⁷. In embodiments, WSG is -(unsubstituted 2 to 10 membered heteroalkylene)-R³³—R³⁷, and R³³ is unsubstituted 5 to 10 membered heteroarylene. In embodiments, WSG is -(unsubstituted 2 to 10 membered heteroalkylene)-R³³—R³⁷, and R³³ is unsubstituted 5 to 10 membered heteroarylene and R³⁷ is -(unsubstituted C₁-C₆ alkyl)-(R⁴⁻¹-substituted or unsubstituted 3 to 10 membered heterocycloalkyl).

In embodiments, WSG is —(C₁-C₁₀ heteroalkyl)-R³³—R³⁷. In embodiments, WSG is —(C₁-C₁₀ heteroalkyl)-R³³—R³⁷ and R³³ is C₁-C₁₀ heteroarylene. In embodiments, WSG is —(C₁-C₁₀ heteroalkyl)-R³³—R³⁷ and R³³ is C₁-C₁₀ heteroarylene and R³⁷ is —(C₁-C₆alkyl)(C₁-C₁₀ heterocycloalkyl).

In embodiments, WSG is

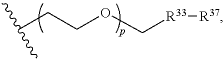

and p is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In embodiments, p is an integer of value 1-10, 1-20, 1-30, 1-40, 1-50, 10-20, 10-30, 10-40, 10-50, 20-30, 20-40, 20-50, 30-40, 30-50, or 40-50. In embodiments, p is 1, 2, 3, 4, 5, 6, 7, 8, 9, or 10. In embodiments, p is 3 or 6.

In embodiments, WSG is

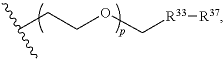

and p is 3.

In embodiments, WSG is

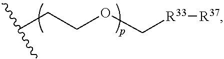

and R³³ is an unsubstituted 5 to 10 membered heteroarylene. In embodiments, WSG is

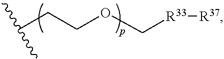

and R³³ is an unsubstituted 5 membered heteroarylene. In embodiments, WSG is

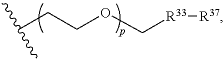

and R³³ is unsubstituted triazolylene, unsubstituted imidazolylene, or unsubstituted pyrazolylene. In embodiments, WSG is

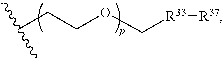

and R³³ is unsubstituted triazolylene. In embodiments, WSG is

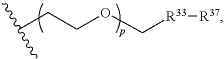

and R³³ is unsubstituted 1,2,4-triazolylene. In embodiments, WSG is

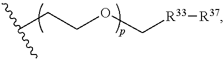

and R³³ is unsubstituted 1,2,3-triazolylene. In embodiments, WSG is

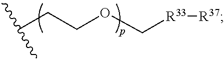

R³³ is unsubstituted 1,2,3-triazolylene, and p is 3. In embodiments, WSG is

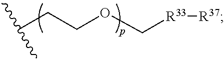

R³ is unsubstituted 1,2,3-triazolylene and R³⁷ is -(unsubstituted C₁-C₆ alkylene)-(R⁴¹-substituted or unsubstituted 3 to 10 membered heterocycloalkyl). In embodiments, WSG is

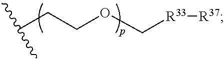

R³³ is unsubstituted 1,2,3-triazolylene and R³⁷ is —CH₂—(R⁴¹-substituted or unsubstituted 3 to 10 membered heterocycloalkyl). In embodiments, WSG is

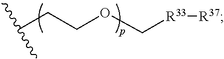

R³³ is unsubstituted 1,2,3-triazolylene and R³⁷ is R⁴¹-substituted or unsubstituted tetrahydropyranyl. In embodiments, WSG is

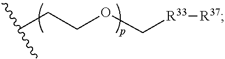

R³³ is unsubstituted 1,2,3-triazolylene, and R³⁷ is

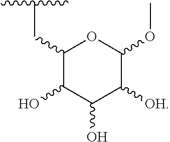 [SMILES: *CC1OC(OC)C(O)C(O)C1O]

In embodiments, WSG is

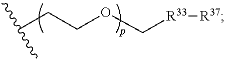

R³³ is unsubstituted 1,2,3-triazolylene, R³⁷ is

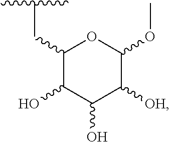 [SMILES: *CC1OC(OC)C(O)C(O)C1O]

and p is 3.

In embodiments, WSG is

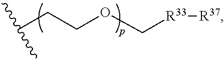

and R³³ is a C₁-C₁₀ heteroarylene.

In embodiments, WSG is

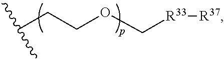

and R³³ is a C₅ heteroarylene.

In embodiments, WSG is

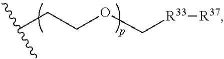

and R³³ is triazole, imidazole, or pyrazole.

In embodiments, WSG is

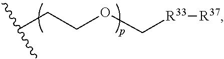

and R³³ is triazole.

In embodiments, WSG is

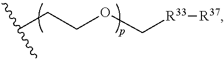

and R³³ is 1,2,4-triazole. In embodiments, WSG is

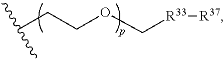

and R³³ is 1,2,3-triazole.

In embodiments, WSG is

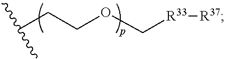

R³³ is 1,2,3-triazole, and p is 3.

In embodiments, WSG is

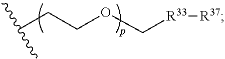

R³³ is 1,2,3-triazole and R³⁷ is —(C₁-C₆alkyl)(C₁-C₁₀ heterocycloalkyl).

In embodiments, WSG is

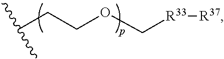

R³³ is 1,2,3-triazole and R³⁷ is —(C₁alkyl)(C₁-C₁₀ heterocycloalkyl).

In embodiments, WSG is

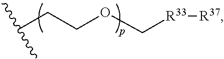

R³³ is 1,2,3-triazole, R³⁷ is a tetrahydropyran derivative.

In embodiments, WSG is

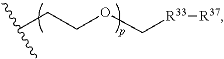

R³³ is 1,2,3-triazole, and R³⁷ is

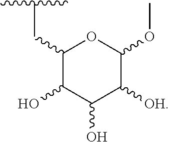 [SMILES: *CC1OC(OC)C(O)C(O)C1O]

In embodiments, WSG is

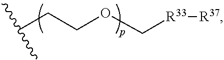

R³³ is 1,2,3-triazole, R³⁷ is

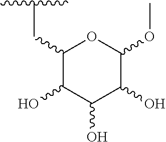 [SMILES: *CC1OC(OC)C(O)C(O)C1O]

and p is 3.

In embodiments, WSG is

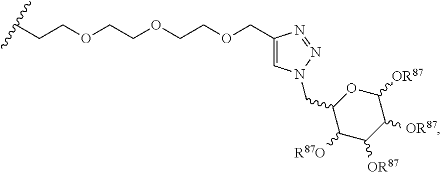

wherein each R⁸⁷ is independently hydrogen, unsubstituted alkyl (e.g., C₁-C₈, C₁-C₆, C₁-C₄, or C₁-C₂), or —C(═O)-(unsubstituted alkyl). In embodiments, WSG is

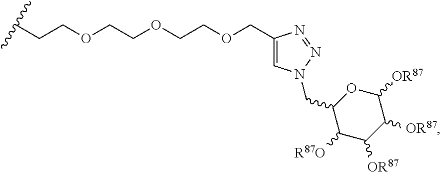

wherein each R⁸⁷ is independently hydrogen, unsubstituted C₁-C₁₀ alkyl, or —C(═O)-(unsubstituted C₁-C₁₀ alkyl). In embodiments, R⁸⁷ is independently hydrogen. In embodiments, R⁸⁷ is independently unsubstituted methyl. In embodiments, R⁸⁷ is independently unsubstituted ethyl. In embodiments, R⁸⁷ is independently unsubstituted propyl. In embodiments, R⁸⁷ is independently unsubstituted butyl. In embodiments, R⁸⁷ is independently unsubstituted acetyl. In embodiments, R⁸⁷ is independently unsubstituted propionyl. In embodiments, R⁸⁷ is independently unsubstituted butyryl. In embodiments, R⁸⁷ is independently hydrogen or methyl. In embodiments, R⁸⁷ is independently methyl or acetyl.

In embodiments, WSG

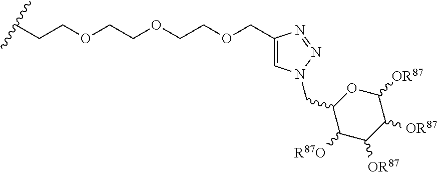

wherein each R⁸⁷ is hydrogen, C₁-C₁₀ alkyl, or —C(═O)C₁-C₁₀ alkyl. In embodiments, each R⁸⁷ is independently a hydrogen, methyl, ethyl, propyl, butyl, acetate, propionate, or butyrate. In embodiments, each R⁸⁷ is independently a hydrogen or methyl. In embodiments, each R⁸⁷ is independently a methyl or acetate.

In embodiments, WSG is

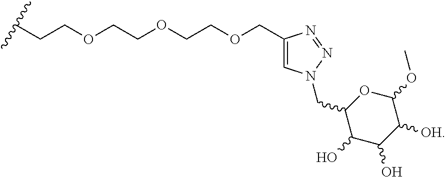 [SMILES: *CCOCCOCCOCc1cn(CC2OC(OC)C(O)C(O)C2O)nn1]

In embodiments, WSG is

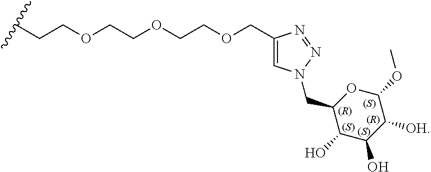

In embodiments, WSG is unsubstituted heteroalkyl. In embodiments, WSG is 2 to 20 membered unsubstituted heteroalkyl. In embodiments, WSG is 2 to 18 membered unsubstituted heteroalkyl. In embodiments, WSG is 2 to 16 membered unsubstituted heteroalkyl. In embodiments, WSG is 2 to 14 membered unsubstituted heteroalkyl. In embodiments, WSG is 2 to 12 membered unsubstituted heteroalkyl. In embodiments, WSG is 2 to 10 membered unsubstituted heteroalkyl. In embodiments, WSG is 2 to 8 membered unsubstituted heteroalkyl. In embodiments, WSG is 2 to 6 membered unsubstituted heteroalkyl. In embodiments, WSG is 2 to 4 membered unsubstituted heteroalkyl. In embodiments, the heteroalkyl group contains at least one heteroatom, wherein the heteroatom is oxygen. In embodiments, the heteroalkyl group contains two heteroatoms, wherein the heteroatoms are oxygen. In embodiments, the heteroalkyl group contains three heteroatoms, wherein the heteroatoms are oxygen. In embodiments, the heteroalkyl group contains four heteroatoms, wherein the heteroatoms are oxygen.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R¹ substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.1} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW2}, and R^{WW.3} correspond to R⁵, R^{5.1}, R^{5.2}, and R^{5.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R¹⁴ is substituted, R¹⁴ is substituted with one or more first substituent groups denoted by R^{14.1} as explained in the definitions section above in the description of “first substituent group(s)”. In embodiments, when an R^{14.1} substituent group is substituted, the R^{14.1} substituent group is substituted with one or more second substituent groups denoted by R^{14.2} as explained in the definitions section above in the description of “first substituent group(s)”. In embodiments, when an R^{14.2} substituent group is substituted, the R^{14.2} substituent group is substituted with one or more third substituent groups denoted by R^{14.3} as explained in the definitions section above in the description of “first substituent group(s)”. In the above embodiments, R¹⁴, R¹⁴, R^{14.2}, and R^{14.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁴, R^{14.1}, R^{14.2}, and R^{14.3}, respectively.

In embodiments, when R⁸¹ is substituted, R⁸¹ is substituted with one or more first substituent groups denoted by R^{81.1} as explained in the definitions section above in the description of “first substituent group(s)”. In embodiments, when an R^{81.1} substituent group is substituted, the R^{81.1} substituent group is substituted with one or more second substituent groups denoted by R^{81.2} as explained in the definitions section above in the description of “first substituent group(s)”. In embodiments, when an R^{81.2} substituent group is substituted, the R^{81.2} substituent group is substituted with one or more third substituent groups denoted by R^{81.3} as explained in the definitions section above in the description of “first substituent group(s)”. In the above embodiments, R⁸¹, R^{81.1}, R^{81.2}, and R^{81.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸¹, R^{81.1}, R^{81.2}, and R^{81.3}, respectively.

In embodiments, when R⁸² is substituted, R⁸² is substituted with one or more first substituent groups denoted by R^{82.1} as explained in the definitions section above in the description of “first substituent group(s)”. In embodiments, when an R^{82.1} substituent group is substituted, the R^{82.1} substituent group is substituted with one or more second substituent groups denoted by R^{82.2} as explained in the definitions section above in the description of “first substituent group(s)”. In embodiments, when an R^{82.2} substituent group is substituted, the R^{82.2} substituent group is substituted with one or more third substituent groups denoted by R^{82.3} as explained in the definitions section above in the description of “first substituent group(s)”. In the above embodiments, R⁸², R^{82.1}, R^{82.2}, and R^{82.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸², R^{82.1}, R^{82.2}, and R^{82.3}, respectively.

In embodiments, when R⁸³ is substituted, R⁸³ is substituted with one or more first substituent groups denoted by R^{83.1} as explained in the definitions section above in the description of “first substituent group(s)”. In embodiments, when an R^{83.1} substituent group is substituted, the R^{83.1} substituent group is substituted with one or more second substituent groups denoted by R^{83.2} as explained in the definitions section above in the description of “first substituent group(s)”. In embodiments, when an R^{83.2} substituent group is substituted, the R^{83.2} substituent group is substituted with one or more third substituent groups denoted by R^{83.3} as explained in the definitions section above in the description of “first substituent group(s)”. In the above embodiments, R⁸³, R^{83.1}, R^{83.2}, and R^{83.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸³, R^{83.1}, R^{83.2}, and R⁸³³, respectively.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

In embodiments, the compound of Formula Ia (e.g., Formula I) binds alpha-synuclein more strongly than the compound of Formula Ia (e.g., Formula I) binds amyloid beta under identical conditions. In embodiments, the compound of Formula Ia (e.g., Formula I) binds alpha-synuclein at least 2-fold stronger than the compound of Formula Ia (e.g., Formula I) binds amyloid beta under identical conditions. In embodiments, the compound of Formula Ia (e.g., Formula I) binds alpha-synuclein at least 5-fold stronger than the compound of Formula Ia (e.g., Formula I) binds amyloid beta under identical conditions. In embodiments, the compound of Formula Ta (e.g., Formula I) binds alpha-synuclein at least 10-fold stronger than the compound of Formula Ta (e.g., Formula I) binds amyloid beta under identical conditions. In embodiments, the compound of Formula Ia (e.g., Formula I) binds alpha-synuclein at least 20-fold stronger than the compound of Formula Ia (e.g., Formula I) binds amyloid beta under identical conditions. In embodiments, the compound of Formula Ia (e.g., Formula I) binds alpha-synuclein at least 40-fold stronger than the compound of Formula Ia (e.g., Formula I) binds amyloid beta under identical conditions. In embodiments, the compound of Formula Ia (e.g., Formula I) binds alpha-synuclein at least 60-fold stronger than the compound of Formula Ia (e.g., Formula I) binds amyloid beta under identical conditions. In embodiments, the compound of Formula Ia (e.g., Formula I) binds alpha-synuclein at least 80-fold stronger than the compound of Formula Ia (e.g., Formula I) binds amyloid beta under identical conditions. In embodiments, the compound of Formula Ia (e.g., Formula I) binds alpha-synuclein at least 100-fold stronger than the compound of Formula Ta (e.g., Formula I) binds amyloid beta under identical conditions. In embodiments, the compound of Formula Ia (e.g., Formula I) binds alpha-synuclein at least 500-fold stronger than the compound of Formula Ia (e.g., Formula I) binds amyloid beta under identical conditions.

In embodiments, the compound of Formula IIa (e.g., Formula II) binds amyloid beta more strongly than the compound of Formula IIa (e.g., Formula II) binds alpha-synuclein under identical conditions. In embodiments, the compound of Formula IIa (e.g., Formula II) binds amyloid beta at least 2-fold stronger than the compound of Formula IIa (e.g., Formula II) binds alpha-synuclein under identical conditions. In embodiments, the compound of Formula IIa (e.g., Formula II) binds amyloid beta at least 5-fold stronger than the compound of Formula IIa (e.g., Formula II) binds alpha-synuclein under identical conditions. In embodiments, the compound of Formula IIa (e.g., Formula II) binds amyloid beta at least 10-fold stronger than the compound of Formula IIa (e.g., Formula II) binds alpha-synuclein under identical conditions. In embodiments, the compound of Formula IIa (e.g., Formula II) binds amyloid beta at least 20-fold stronger than the compound of Formula IIa (e.g., Formula II) binds alpha-synuclein under identical conditions. In embodiments, the compound of Formula IIa (e.g., Formula II) binds amyloid beta at least 40-fold stronger than the compound of Formula IIa (e.g., Formula II) binds alpha-synuclein under identical conditions. In embodiments, the compound of Formula IIa (e.g., Formula II) binds amyloid beta at least 60-fold stronger than the compound of Formula IIa (e.g., Formula II) binds alpha-synuclein under identical conditions. In embodiments, the compound of Formula IIa (e.g., Formula II) binds amyloid beta at least 80-fold stronger than the compound of Formula IIa (e.g., Formula II) binds alpha-synuclein under identical conditions. In embodiments, the compound of Formula IIa (e.g., Formula II) binds amyloid beta at least 100-fold stronger than the compound of Formula IIa (e.g., Formula II) binds alpha-synuclein under identical conditions.

In embodiments, the compound of Formula IIa (e.g., Formula II) binds amyloid beta at least 500-fold stronger than the compound of Formula IIa (e.g., Formula II) binds alpha-synuclein under identical conditions.

In an aspect is provided a composition including a compound as described herein, or a pharmaceutically acceptable salt thereof, and an amyloid or amyloid like protein. In embodiments, the amyloid or amyloid like protein is Aβ peptide. In embodiments, the amyloid or amyloid like protein is prion peptide. In embodiments, the amyloid or amyloid like protein is alpha-synuclein. In embodiments, the amyloid or amyloid like protein is superoxide dismutase. In embodiments, the amyloid or amyloid like protein is tau. In embodiments, the amyloid or amyloid like protein is phosphorylated tau. In embodiments, the amyloid or amyloid like protein is TDP-43. In embodiments, the amyloid or amyloid like protein is TMEM106b. In embodiments, the amyloid or amyloid like protein is beta amyloid (1-42) (AD (1-42)).

### Iv. Routes of Administration

In embodiments, the formulations of the present disclosure draw upon many suitable modes of administration. In embodiments, delivery is achieved either via local or systemic administration. Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections. In embodiments, the compounds of the disclosure are administered in a systemic manner. In embodiments, administration is parenteral. In embodiments, the administration is intravenous. In embodiments, the administration is subcutaneous. In embodiments, the administration is intramuscular. In embodiments, the administration is intrathecal. Administration can take place via enteral administration (absorption of the drug through the gastrointestinal tract) or parenteral administration, for example by injection, infusion, or implantation. In embodiments, administration is transmucosal, such as oral, buccal, sublingual, nasal, pulmonary, or rectal. In embodiments, administration is oral. In embodiments, administration is buccal. In embodiments, administration is sublingual. In embodiments, administration is nasal. In embodiments, administration is pulmonary. In embodiments, administration is rectal. In embodiments, administration is transdermal. In embodiments, administration is intradermal. In embodiments, administration is topica. In embodiments, administration is topical ocular.

In certain cases, a compound as described herein is administered in a local rather than systemic manner, for example, via injection of the compound directly into an organ, often in a depot preparation or sustained release formulation. In specific cases, long acting formulations are administered by implantation (for example subcutaneously or intramuscularly) or by intramuscular injection. Furthermore, in other cases, the drug is delivered in a targeted drug delivery system, for example, in a liposome coated with organ-specific antibody. In such cases, the liposomes are targeted to and taken up selectively by the organ. In yet other cases, the compound as described herein is provided in the form of a rapid release formulation, in the form of an extended release formulation, or in the form of an intermediate release formulation. In yet other cases, the compound described herein is administered topically.

In embodiments, the compounds are administered to the eye. In embodiments, the pharmaceutical composition of the disclosure administered to eye is delivered to the retina, intraocular space, ocular surface, interconnecting innervation, conjunctiva, lacrimal glands, or meibomian glands. In embodiments, the compounds are administered topically to the eye. In embodiments, the compounds are administered as an eye drop.

The compounds according to the disclosure are effective over a wide dosage range. In embodiments, in the treatment of adult humans, dosages are from 0.01 to 1000 mg, from 0.5 to 100 mg, from 1 to 50 mg per day, and from 5 to 40 mg per day are examples of dosages that are used. An exemplary dosage is 10 to 30 mg per day. In embodiments, the effective amount the compound corresponds to about 50-500 mg of compound per adult subject. The exact dosage will depend upon the route of administration, the form in which the compound is administered, the subject to be treated, the body weight of the subject to be treated, and the preference and experience of the attending physician.

In embodiments, the effective amount the compound corresponds to about 0.01-1000 mg of compound per adult human subject per dosage. In embodiments, the effective dose of compound is be 50-500 mg per adult human per dosage. In embodiments, the effective amount corresponds to about 0.01-100 mg, 0.01-200 mg, 0.01-300 mg, 0.01-400 mg, 0.01-500 mg, 0.01-600 mg, 0.01-700 mg, 0.01-800 mg, 0.01-900 mg, 0.01-1000 mg, 0.1-100 mg, 0.1-200 mg, 0.1-300 mg, 0.1-400, 0.1-500 mg, 0.1-600 mg, 0.1-700 mg, 0.1-800 mg, 0.1-900 mg, 0.1-1000 mg, 1-100 mg, 1-200 mg, 1-300 mg, 1-400 mg, 1-500 mg, 1-600 mg, 1-700 mg, 1-800 mg, 1-900 mg, 100-200 mg, 100-300 mg, 100-400 mg, 100-500 mg, 100-600 mg, 100-700 mg, 100-800 mg, 100-900 mg, 100-1000 mg, 200-300 mg, 200-400 mg, 200-500 mg, 200-600 mg, 200-700 mg, 200-800 mg, 200-900 mg, 200-1000 mg, 300-400 mg, 300-500 mg, 300-600 mg, 300-700 mg, 300-800 mg, 300-900 mg, 300-1000 mg, 400-500 mg, 400-600 mg, 400-700 mg, 400-800 mg, 400-900 mg, 400-1000 mg, 500-600 mg, 500-700 mg, 500-800 mg, 500-900 mg, 500-1000 mg, 600-700 mg, 600-800 mg, 600-900 mg, 600-1000 mg, 700-800 mg, 700-900 mg, 700-1000 mg, 800-900 mg, 800-1000 mg or about 900-1000 mg per adult human per dosage. In embodiments, the effective amount corresponds to about 50-100 mg, 50-400 mg, 50-500 mg, 100-200 mg, 100-300 mg, 100-400 mg, 100-500 mg, 200-300 mg, 200-400 mg, 200-500, 300-400 mg, 300-500 mg, or 400-500 mg per adult human per dosage. The exact dosage will depend upon the route of administration, the form in which the compound is administered, the subject to be treated, the body weight of the subject to be treated, and the preference and experience of the attending physician.

In embodiments, a compound of the disclosure is administered in a single dose. In embodiments, a compound of the disclosure is administered in multiple doses. In embodiments, dosing is about once, twice, three times, four times, five times, six times, or more than six times per day. In embodiments, dosing is about once a month, once every two weeks, once a week, or once every other day. In another case a compound of the disclosure and another agent are administered together about once per day to about 6 times per day. In embodiments the administration of a compound of the disclosure and an agent continues for less than about 7 days. In yet another case, the administration continues for more than about 6, 10, 14, 28 days, two months, six months, or one year. In embodiments, continuous dosing is achieved and maintained as long as necessary.

In embodiments, administration of the compounds of the disclosure continues as long as necessary. In embodiments, a compound of the disclosure is administered for more than 1, 2, 3, 4, 5, 6, 7, 14, or 28 days. In embodiments, a compound of the disclosure is administered for less than 28, 14, 7, 6, 5, 4, 3, 2, or 1 day. In embodiments, a compound of the disclosure is administered chronically on an ongoing basis, e.g., for the treatment of chronic effects.

In embodiments, for administration to the eyes, compounds are administered several times a day per eye. In embodiments, the compounds are administered one to ten times, one to four times, or once a day. In embodiments, the compounds are administered 1, 2, 3, 4, 5, 6, 7, 8, 9, or 10 times a day. In embodiments, the size of the drop administered is in the range of about 10-100 μL, about 10-90 μL, about 10-80 μL, about 10-70 μL, about 10-60 μL, about 10-50 μL, about 10-40 μL, about 10-30 μL, about 20-100 μL, about 20-90 μL, about 20-80 μL, about 20-70 μL, about 20-60 μL, about 20-50 μL, about 20-40 μL, or about 20-30 μL. One example of the disclosure administers a drop in the range of about 10 to about 30 μL. One example of the disclosure administers a drop in the range of about 10 to about 100 μL. One example of the disclosure administers a drop in the range of about 20 to about 50 μL. One example of the disclosure administers a drop in the range of about 20 to about 40 μL. One example of the disclosure administers a drop in the range of about 10 to about 60 μL. In embodiments, the eye formulations of the disclosure is administered several drops per time, for example 1-3 drops per time, 1-3 drops per time, 1-4 drops per time, 1-5 drops per time, 1-6 drops per time, 1-7 drops per time, 1-8 drops per time, 1-9 drops per time, 1-10 drops per time, 3-4 drops per time, 3-5 drops per time, 3-6 drops per time, 3-7 drops per time, 3-8 drops per time, 3-9 drops per time, 3-10 drops per time, 5-6 drops per time, 5-7 drops per time, 5-8 drops per time, 5-9 drops per time, 5-10 drops per time, 7-8 drops per time, 7-9 drops per time or 9-10 drops per time. In one example, the formulations of the disclosure are administered about one drop per time and 1-6 times per day.

In embodiments, the compounds of the disclosure are administered in dosages. It is known in the art that due to intersubject variability in compound pharmacokinetics, individualization of dosing regimen is necessary for optimal therapy. In embodiments, dosing for a compound of the disclosure is found by routine experimentation in light of the instant disclosure.

### V. Pharmaceutical Compositions/Formulations

In an aspect is provided a pharmaceutical composition including a compound described herein, or a pharmaceutically acceptable salt thereof, and a pharmaceutically acceptable excipient.

In embodiments, the pharmaceutically acceptable excipient is ethanol, dimethylsulfoxide, polyethylene glycol, polypropylene glycol, aqueous acetate buffer, aqueous citrate buffer, aqueous phosphate buffer, aqueous carbonate buffer, cyclodextrin, corn oil, vitamin E, polysorbate, bile acid, or a combination of two or more thereof. In embodiments, the pharmaceutically acceptable excipient is ethanol. In embodiments, the pharmaceutically acceptable excipient is dimethylsulfoxide. In embodiments, the pharmaceutically acceptable excipient is polyethylene glycol. In embodiments, the pharmaceutically acceptable excipient is polypropylene glycol. In embodiments, the pharmaceutically acceptable excipient is aqueous acetate buffer. In embodiments, the pharmaceutically acceptable excipient is aqueous citrate buffer. In embodiments, the pharmaceutically acceptable excipient is aqueous phosphate buffer. In embodiments, the pharmaceutically acceptable excipient is aqueous carbonate buffer. In embodiments, the pharmaceutically acceptable excipient is cyclodextrin. In embodiments, the pharmaceutically acceptable excipient is corn oil. In embodiments, the pharmaceutically acceptable excipient is vitamin E. In embodiments, the pharmaceutically acceptable excipient is polysorbate. In embodiments, the pharmaceutically acceptable excipient is bile acid.

In embodiments, the compounds described herein are formulated into pharmaceutical compositions. In embodiments, pharmaceutical compositions are formulated in a conventional manner using one or more physiologically acceptable carriers comprising excipients and auxiliaries, which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein, inter alia, are pharmaceutical compositions comprising a compound of Formula Ia or Formula IIa (e.g., Formula I or Formula II) and a pharmaceutically acceptable diluent(s), excipient(s), or carrier(s). In certain cases, the compounds described are administered as pharmaceutical compositions in which compounds of any of Formula Ia or Formula IIa (e.g., Formula I or Formula II), are mixed with other active ingredients, as in combination therapy. In specific cases, the pharmaceutical compositions include one or more compounds of any of Formula Ia or Formula IIa (e.g., Formula I or Formula II).

A pharmaceutical composition, as used herein, refers to a mixture of a compound of any of Formula Ia or Formula IIa (e.g., Formula I or Formula II), with other chemical components, such as carriers, stabilizers, diluents, dispersing agents, suspending agents, thickening agents, and/or excipients. In certain cases, the pharmaceutical composition facilitates administration of the compound to an organism. In embodiments, practicing the methods of treatment or use provided herein, therapeutically effective amounts of compounds of any of Formula Ia or Formula IIa (e.g., Formula I or Formula II), provided herein are administered in a pharmaceutical composition to a mammal having a disease or condition to be detected, diagnosed or treated. In specific cases, the mammal is a human. In certain cases, therapeutically effective amounts vary depending on the severity of the disease, the age and relative health of the subject, the potency of the compound used and other factors. The compounds described herein are used singly or in combination with one or more therapeutic agents as components of mixtures.

In embodiments, one or more compounds of any of Formula Ia or Formula IIa (e.g., Formula I or Formula II) is formulated in an aqueous solution. In specific cases, the aqueous solution is selected from, by way of example only, a physiologically compatible buffer, such as Hank's solution, Ringer's solution, aqueous acetate buffer, aqueous citrate buffer, aqueous carbonate buffer, aqueous phosphate buffer or physiological saline buffer.

In other cases, one or more compound of any of Formula Ta or Formula IIa (e.g., Formula I or Formula II) is formulated for transmucosal administration. In specific cases, transmucosal formulations include penetrants that are appropriate to the barrier to be permeated. In still other cases wherein the compounds described herein are formulated for other parenteral injections, appropriate formulations include aqueous or nonaqueous solutions. In specific cases, such solutions include physiologically compatible buffers and/or excipients.

In embodiments, the compounds described herein are formulated for ocular administration. In embodiments, the ocular formulations is liquid (in form of solutions, suspensions, powder for reconstitution, sol to gel systems), semi solids (ointments and gels), solids (ocular inserts), and intraocular dosage forms (injections, irrigating solutions and implants).

In another case, compounds described herein are formulated for oral administration. Compounds described herein, including compounds of any of Formula Ia or Formula IIa (e.g., Formula I or Formula II), are formulated by combining the active compounds with, e.g., pharmaceutically acceptable carriers or excipients. In various cases, the compounds described herein are formulated in oral dosage forms that include, by way of example only, tablets, powders, pills, dragees, capsules, liquids, gels, syrups, elixirs, slurries, suspensions and the like.

In certain cases, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific cases, disintegrating agents are optionally added. Disintegrating agents include, by way of example only, cross-linked croscarmellose sodium, polyvinylpyrrolidone, agar, or alginic acid or a salt thereof such as sodium alginate.

In embodiments, dosage forms, such as dragee cores and tablets, are provided with one or more suitable coating. In specific cases, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain cases, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific cases, push-fit capsules contain the active ingredients in admixture with one or more filler. Fillers include, by way of example only, lactose, binders such as starches, and/or lubricants such as talc or magnesium stearate and, optionally, stabilizers. In other cases, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other cases, therapeutically effective amounts of at least one of the compounds described herein are formulated for buccal or sublingual administration. Formulations suitable for buccal or sublingual administration include, by way of example only, tablets, lozenges, or gels. In still other cases, the compounds described herein are formulated for parental injection, including formulations suitable for bolus injection or continuous infusion. In specific cases, formulations for injection are presented in unit dosage form (e.g., in ampoules) or in multi-dose containers. Preservatives are, optionally, added to the injection formulations. In still other cases, the pharmaceutical composition of any of Formula Ia or Formula IIa (e.g., Formula I or Formula II), are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific cases, pharmaceutical formulations for parenteral administration include aqueous solutions of the active compounds in water-soluble form. In additional cases, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific cases, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other cases, the active ingredient is in powder form for constitution with a suitable vehicle, e.g., sterile pyrogen-free water, before use.

In still other cases, the compounds of any of Formula Ia or Formula IIa (e.g., Formula I or Formula II) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other cases, the compounds of any of Formula Ia or Formula IIa (e.g., Formula I or Formula II) are formulated for transdermal administration. In specific cases, transdermal formulations employ transdermal delivery devices and transdermal delivery patches and can be lipophilic emulsions or buffered, aqueous solutions, dissolved and/or dispersed in a polymer or an adhesive. In various cases, such patches are constructed for continuous, pulsatile, or on demand delivery of pharmaceutical agents. In additional cases, the transdermal delivery of the compounds of any of Formula Ia or Formula IIa (e.g., Formula I or Formula II) is accomplished by means of iontophoretic patches and the like. In certain cases, transdermal patches provide controlled delivery of the compounds of any of Formula Ia or Formula IIa (e.g., Formula I or Formula II). In specific cases, the rate of absorption is slowed by using rate-controlling membranes or by trapping the compound within a polymer matrix or gel. In alternative cases, absorption enhancers are used to increase absorption. Absorption enhancers or carriers include absorbable pharmaceutically acceptable solvents that assist passage through the skin. In embodiments, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other cases, the compounds of any of Formula Ia or Formula IIa (e.g., Formula I or Formula II) are formulated for administration by inhalation. Various forms suitable for administration by inhalation include, but are not limited to, aerosols, mists or powders. Pharmaceutical compositions of any of Formula Ia or Formula IIa (e.g., Formula I or Formula II) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific cases, the dosage unit of a pressurized aerosol is determined by providing a valve to deliver a metered amount. In certain cases, capsules and cartridges of, such as, by way of example only, gelatin for use in an inhaler or insufflator are formulated containing a powder mix of the compound and a suitable powder base such as lactose or starch.

In still other cases, the compounds of any of Formula Ia or Formula IIa (e.g., Formula I or Formula II) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain cases, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of any of Formula Ia or Formula IIa (e.g., Formula I or Formula II) are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of any of Formula Ia or Formula IIa (e.g., Formula I or Formula II) described herein as an active ingredient. The active ingredient is in free-acid or free-base form, or in a pharmaceutically acceptable salt form. In addition, the methods and pharmaceutical compositions described herein include the use ofN-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In embodiments, pharmaceutical composition comprising at least one compound of any of Formula Ia or Formula IIa (e.g., Formula I or Formula II) illustratively takes the form of a liquid where the agents are present in solution, in suspension or both. Typically, when the composition is administered as a solution or suspension a first portion of the agent is present in solution and a second portion of the agent is present in particulate form, in suspension in a liquid matrix. In embodiments, a liquid composition includes a gel formulation. In other cases, the liquid composition is aqueous.

In certain cases, useful aqueous suspension contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of any of Formula Ta or Formula IIa (e.g., Formula I or Formula II). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In embodiments, the formulations of the disclosure is packaged in multidose form or in single dose units. In embodiments, the formulations are packaged in multidose forms. In embodiments, the formulations are packaged as single dose from. In embodiments of the disclosure, single dose packaging of the formulations can offer several advantages over multi dose packaging including dosage control, increased patient compliance, improved product labeling, and reduced counterfeiting. In various cases, single dosage packaging of the formulations of the disclosure can be in form of vials, ampoules, tubes, bottles, pouches, packets, syringes or blister packs.

In alternative cases, other delivery systems for hydrophobic pharmaceutical compounds are employed. Liposomes and emulsions are examples of delivery vehicles or carriers useful herein. In certain cases, organic solvents such as N-methylpyrrolidone are also employed. In additional cases, the compounds described herein are delivered using a sustained-release system, such as semipermeable matrices of solid hydrophobic polymers containing the therapeutic agent. Various sustained-release materials are useful herein. In embodiments, sustained-release capsules release the compounds for a few weeks up to over 100 days. Depending on the chemical nature and the biological stability of the therapeutic reagent, additional strategies for protein stabilization are employed.

In certain cases, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In embodiments, the concentration of one or more compounds provided in the pharmaceutical compositions of the present disclosure is less than 100%, 90%, 80%, 70%, 60%, 50, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In embodiments, the concentration of one or more compounds of the disclosure is greater than 90% 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In embodiments, the concentration of one or more compounds of the disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. In embodiments, the concentration of one or more compounds of the disclosure is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In embodiments, the amount of one or more compounds of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g. In embodiments, the amount of one or more compounds of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g. In embodiments, the amount of one or more compounds of the disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

### VI. Kits/Articles of Manufacture

The disclosure also provides a kit comprising a compound according to the disclosure. In embodiments, the compounds of the disclosure are contained in a container as formulations. In embodiments, the kit comprises the compounds of the disclosure contained in a container as a sterile liquid formulation. In embodiments, the compounds are also placed in the containers as a sterile freeze-dried formulation. In embodiments, the container is a vial. In embodiments, the container is an amber vial. In embodiments, the container is capable of protecting light sensitive compounds or formulation.

In embodiments, such kits comprise a carrier, package, or container that is compartmentalized to receive one or more containers such as vials, tubes, and the like, wherein one or more of the container(s) comprise the compound of Formula Ia or Formula IIa (e.g., Formula I or Formula II). Suitable containers include, for example, bottles, vials, syringes, and test tubes. The containers are formed from a variety of materials such as glass or plastic. In embodiments, the containers are chosen so as to protect, limit or minimize the exposure of the compounds of Formula Ia or Formula IIa (e.g., Formula I or Formula II) to light. In embodiments, the container is an amber vial.

The articles of manufacture provided herein contain packaging materials. Packaging materials for use in packaging pharmaceutical products Include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

In embodiments, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain cases, the pharmaceutical composition is presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In embodiments, Compositions containing a compound provided herein formulated in a compatible pharmaceutical carrier are prepared, placed in an appropriate container, and labeled for treatment of an indicated condition.

### VII. Methods of Use

In one aspect, the disclosure provides a method for detecting one or more amyloid or amyloid like proteins comprising contacting a compound according to according to any one of Formula Ia or Formula IIa (e.g., Formula I or Formula II) or a pharmaceutical composition thereof with a sample potentially comprising the amyloid or amyloid like protein, wherein in presence of an amyloid or amyloid like protein the compound forms a detectable complex, detecting the formation of the detectable complex such that the presence or absence of the detectable complex correlates with the presence or absence of the amyloid or amyloid like protein. In embodiments, the sample is derived from a subject (e.g., human subject) having or suspected of having an amyloid-based disease or condition. In embodiments, the sample is derived from a subject (e.g., human subject) having an amyloid-based disease or condition. In embodiments, the sample is derived from a subject (e.g., human subject) suspected of having an amyloid-based disease or condition.

In embodiments, the detection of the formation of the detectable complex is performed by measuring a signal generated by the detectable complex. In embodiments, the signal generated by the detectable complex is an electromagnetic signal. In embodiments, the electromagnetic signal is a fluorescence signal. In embodiments, the fluorescence signal is measured at a wavelength of from 250 nm to 700 nm. In embodiments, the fluorescence signal is measured at a wavelength of from 450 nm to 650 nm. In embodiments, the fluorescence signal is measured at a wavelength of from 520 nm to 540 nm. In embodiments, the fluorescence signal is measured at a wavelength of 250 nm. In embodiments, the fluorescence signal is measured at a wavelength of 275 nm. In embodiments, the fluorescence signal is measured at a wavelength of 300 nm. In embodiments, the fluorescence signal is measured at a wavelength of 325 nm. In embodiments, the fluorescence signal is measured at a wavelength of 350 nm. In embodiments, the fluorescence signal is measured at a wavelength of 375 nm. In embodiments, the fluorescence signal is measured at a wavelength of 400 nm. In embodiments, the fluorescence signal is measured at a wavelength of 425 nm. In embodiments, the fluorescence signal is measured at a wavelength of 450 nm. In embodiments, the fluorescence signal is measured at a wavelength of 475 nm. In embodiments, the fluorescence signal is measured at a wavelength of 500 nm. In embodiments, the fluorescence signal is measured at a wavelength of 510 nm. In embodiments, the fluorescence signal is measured at a wavelength of 520 nm. In embodiments, the fluorescence signal is measured at a wavelength of 530 nm. In embodiments, the fluorescence signal is measured at a wavelength of 540 nm. In embodiments, the fluorescence signal is measured at a wavelength of 550 nm. In embodiments, the fluorescence signal is measured at a wavelength of 575 nm. In embodiments, the fluorescence signal is measured at a wavelength of 600 nm. In embodiments, the fluorescence signal is measured at a wavelength of 625 nm. In embodiments, the fluorescence signal is measured at a wavelength of 650 nm. In embodiments, the fluorescence signal is measured at a wavelength of 675 nm. In embodiments, the fluorescence signal is measured at a wavelength of 700 nm.

In embodiments, the amyloid or amyloid like protein is Aβ peptide. In embodiments, the amyloid or amyloid like protein is prion peptide. In embodiments, the amyloid or amyloid like protein is alpha-synuclein. In embodiments, the amyloid or amyloid like protein is superoxide dismutase. In embodiments, the amyloid or amyloid like protein is tau. In embodiments, the amyloid or amyloid like protein is phosphorylated tau. In embodiments, the amyloid or amyloid like protein is TDP-43. In embodiments, the amyloid or amyloid like protein is TMEM106b. In embodiments, the amyloid or amyloid like protein is beta amyloid (1-42) (Aβ(1-42)).

In embodiments, the detection of the formation of the detectable complex is performed within about 1 sec, about 5 sec, about 1 min, about 10 min, about 30 min or about 60 min of the contacting of the compound with the sample. In embodiments, the detection of the formation of the detectable complex is performed within about 1-5 minutes of the contacting of the compound with the sample. In embodiments, the detection of the formation of the detectable complex is performed within 1 sec of the contacting of the compound with the sample. In embodiments, the detection of the formation of the detectable complex is performed within 5 sec of the contacting of the compound with the sample. In embodiments, the detection of the formation of the detectable complex is performed within 1 min of the contacting of the compound with the sample. In embodiments, the detection of the formation of the detectable complex is performed within 2 min of the contacting of the compound with the sample. In embodiments, the detection of the formation of the detectable complex is performed within 3 min of the contacting of the compound with the sample. In embodiments, the detection of the formation of the detectable complex is performed within 4 min of the contacting of the compound with the sample. In embodiments, the detection of the formation of the detectable complex is performed within 5 min of the contacting of the compound with the sample. In embodiments, the detection of the formation of the detectable complex is performed within 6 min of the contacting of the compound with the sample. In embodiments, the detection of the formation of the detectable complex is performed within 7 min of the contacting of the compound with the sample. In embodiments, the detection of the formation of the detectable complex is performed within 8 min of the contacting of the compound with the sample. In embodiments, the detection of the formation of the detectable complex is performed within 9 min of the contacting of the compound with the sample. In embodiments, the detection of the formation of the detectable complex is performed within 10 min of the contacting of the compound with the sample. In embodiments, the detection of the formation of the detectable complex is performed within 30 min of the contacting of the compound with the sample. In embodiments, the detection of the formation of the detectable complex is performed within 60 min of the contacting of the compound with the sample.

In embodiments, the compounds of the instant disclosure are used for detecting one or more amyloid or amyloid like protein with high sensitivity. In embodiments, the compounds predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% sensitivity. In embodiments, the compounds are capable of detecting one or more amyloid or amyloid like protein with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity. In embodiments, the compounds are capable of detecting one or more amyloid or amyloid like protein with greater than 99.5%, 99.6%, 99.7%, 99.8% or 99.9% sensitivity.

In embodiments, the compounds of the instant disclosure are used for detecting one or more amyloid or amyloid like protein with high specificity. In embodiments, the compounds detect one or more amyloid or amyloid like protein with greater than 10%, 15%, 20%, 25%, 30%, 35%, 40%, 45%, 50%, 55%, 60%, 65%, 70%, 75%, 80%, 85%, 90%, 95% or 99% specificity. In embodiments, the compounds are capable of detecting one or more amyloid or amyloid like protein with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity. In embodiments, the compounds are capable of detecting one or more amyloid or amyloid like protein with greater than 99.5%, 99.6%, 99.7%, 99.8% or 99.9% specificity.

In embodiments, the compounds of the disclosure are also used for detecting one or more amyloid or amyloid like protein with both high specificity and high specificity. In embodiments, the compounds are capable of detecting one or more amyloid or amyloid like protein with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity and greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity.

The disclosure also provides a method for treating or preventing one or more disease or condition comprising administering to a subject in need of treatment an effective amount of a compound to any one of Formula Ia or Formula IIa (e.g., Formula I or Formula II) or a pharmaceutical composition thereof. In embodiments, the compounds of the disclosure are used to treat or prevent diseases or conditions characterized by protein aggregation or protein misfolding. In embodiments, the disease or condition is an amyloid-based disease or condition.

In embodiments, the compounds of the instant disclosure are used for treating or preventing a disease or condition with high sensitivity. In embodiments, the compounds treat or prevent a disease or condition with greater than 10%, 15%, 20%, 25%, 30%, 35%, 40%, 45%, 50%, 55%, 60%, 65%, 70%, 75%, 80%, 85%, 90%, 95% or 99% sensitivity. In embodiments, the compounds are capable of treating or preventing a disease or condition with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity. In embodiments, the compounds are capable of treating or preventing a disease or condition with greater than 99.5%, 99.6%, 99.7%, 99.8% or 99.9% sensitivity.

In embodiments, the compounds of the instant disclosure are used for treating or preventing a disease or condition with high specificity. In embodiments, the compound predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% specificity. In embodiments the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity. In embodiments the compounds are capable of diagnosis with greater than 99.5%, 99.6%, 99.7%, 99.8% or 99.9% specificity.

In embodiments, the compounds of the disclosure are also used for treating or preventing a disease or condition with both high specificity and high specificity. In embodiments, the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity and greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity.

Also provided herein is a method of determining the presence or absence of one or more disease or condition in a subject comprising administering to the subject an effective amount of a compound according to any one of Formula Ia or Formula IIa (e.g., Formula I or Formula II) or a pharmaceutical composition thereof, wherein in presence of the disease or condition the administered compound forms a detectable complex, and detecting the formation of the detectable complex such that presence or absence of detectable complex correlates with the presence or absence of the disease or condition. In embodiments, the compounds of the disclosure are used for determining the presence or absence of one or more amyloid-based disease or condition, wherein in presence of the amyloid-based disease or condition the administered compound forms a detectable complex, and detecting the formation of the detectable complex such that presence or absence of detectable complex correlates with the presence or absence of the amyloid-based disease or condition. In embodiments, the compounds of the disclosure are used for determining the presence or absence of one or more disease or condition characterized by protein aggregation or protein misfolding.

In embodiments, the method includes comparing the amount of the detectable complex to a normal control value, wherein an increase in the amount of the complex compared to a normal control value indicates that said patient is suffering from or is at risk of developing the disease or condition.

In embodiments, a single dose of the compounds of the disclosure is used to determining the presence or absence of multiple diseases disease or conditions in a subject. In embodiments, a single dose is used to detect the presence or absence of 2, 3, 4, 5, 6, 7, 8, 9, 10, 11, 12, 13, 14, 15, 16, 17, 18, 19 or 20 diseases in a subject. In embodiments, a single dose is used to determine the presence of 1, 2, 3, 4, or 5 disease or conditions.

In embodiments, the compounds of the instant disclosure are used for diagnosis with high sensitivity. In embodiments, the compounds predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% sensitivity. In embodiments, the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity. In embodiments, the compounds are capable of diagnosis with greater than 99.5%, 99.6%, 99.7%, 99.8% or 99.9% sensitivity.

In embodiments, the compounds of the instant disclosure are used for diagnosis with high specificity. In embodiments, the compounds predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% specificity. In embodiments, the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity. In embodiments, the compounds are capable of diagnosis with greater than 99.5%, 99.6%, 99.7%, 99.8% or 99.9% specificity.

In embodiments, the compounds of the disclosure are also used for diagnosis with both high specificity and high specificity. In embodiments, the compounds are capable of diagnosis with greater than 50%, 51%, 52%, 53%, 54%, 55% 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity and greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity.

Also provided herein is a method of monitoring minimal residual disease in a patient following treatment with a compound or a mixture according to the disclosure. The method includes bringing the sample or a specific body part or body area suspected to contain the amyloid or amyloid like protein into contact with a compound of the disclosure, allowing the compound to bind to the amyloid or amyloid like protein to form a detectable complex, detecting the formation of the detectable complex and correlating the presence or absence of the detectable complex with the presence or absence of amyloid or amyloid like protein in the sample or specific body part or area. In embodiments, the method includes comparing the amount of said detectable complex to a normal control value, wherein an increase in the amount of said detectable complex compared to a normal control value indicates that said patient is still be suffering from a minimal residual disease.

Also provided herein is a method of predicting responsiveness of a patient to a treatment, wherein the method includes bringing the sample or a specific body part or body area suspected to contain the amyloid or amyloid like protein into contact with a compound of the disclosure, allowing the compound to bind to the amyloid or amyloid like protein to form a detectable complex, detecting the formation of the detectable complex and correlating the presence or absence of the detectable complex with the presence or absence of amyloid or amyloid like protein in the sample or specific body part or area. In embodiments, the method optionally includes comparing the amount of the detectable complex before and after onset of the treatment, wherein a decrease in the amount of the detectable complex indicates that the patient is being responsive to the treatment. In embodiments, the sample is derived from a subject (e.g., human subject) having or suspected of having an amyloid-based disease or condition. In embodiments, the sample is derived from a subject (e.g., human subject) having an amyloid-based disease or condition. In embodiments, the sample is derived from a subject (e.g., human subject) suspected of having an amyloid-based disease or condition.

Also provided herein is screening method, wherein the method comprises administering to a subject an effective amount of a compound of Formula Ta or Formula IIa (e.g., Formula I or Formula II), or a pharmaceutical composition thereof. In embodiments, upon administration, the compound of Formula Ia or Formula IIa (e.g., Formula I or Formula II) forms a detectable complex. In embodiments, the method further comprises measuring a signal generated by the compound of Formula Ia or Formula IIa (e.g., Formula I or Formula II) upon administration to the subject, or by the detectable complex formed by the compound of Formula Ia or Formula IIa (e.g., Formula I or Formula II). In embodiments, the method also comprises making a clinical decision based on the measured signal.

In embodiments, the detection of the detectable complex disclosure comprises illuminating the sample with light of an appropriate wavelength for a peak region of a fluorescent excitation spectrum for the detectable complex and detecting light received from the sample of an appropriate wavelength for a peak region of a fluorescent emission spectrum for the detectable complex. In embodiments, the detectable complex is a complex of a compound of Formula Ia or Formula IIa (e.g., Formula I or Formula II) with an amyloid or amyloid-like protein. In embodiments, the excitation spectrum has a peak at about 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 n, 490 nm, 500 nm, 510 nm, 520 nm, 530 nm, 540 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, or 900 nm. In embodiments, the fluorescent excitation spectrum of the detectable complex has a peak at about 350-400, 350-450 nm, 350-500 nm, 350-550 nm, 350-600 nm, 400-450 nm, 400-500, 400-550 nm, 400-600 nm, 450-500 nm, 450-550 nm, 450-600 nm, 500-550, or 550-600 nm. In embodiments, the fluorescent excitation spectrum of the detectable complex has a peak at about 350-400 nm, 400-500 nm or 450-500 nm. In embodiments, the illuminating of the sample is at a wavelength within plus or minus about 100 nm, 90 nm, 80 nm, 70 nm, 60 nm, 50 nm, 40 nm, 30 nm, 20 nm, 10 nm, or 0 nm of the peak of the excitation spectrum. In embodiments, the illuminating light has a wavelength of 300-500 nm, 350-450 nm, 400-500 nm. In embodiments, the illuminating light has a wavelength of 400 nm.

In embodiments, the emission spectrum has a peak of about 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 n, 490 nm, 500 nm, 510 nm, 520 nm, 530 nm, 540 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, or 900 nm. In embodiments, the emission spectrum of the detectable complex has a peak at about 500-550 nm, in embodiments at about 510-540 nm. In embodiments, the emission spectrum of the detectable complex has a peak at about 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm or 540 nm. In embodiments, the detecting of light received from the sample is at a wavelength within plus or minus about 100 nm, 90 nm, 80 nm, 70 nm, 60 nm, 50 nm, 40 nm, 30 nm, 20 nm, 10 nm, or 0 nm of the peak of the emission spectrum.

In embodiments, the term amyloid-based disease or condition refers to any disease or condition. The term also includes any disease or condition characterized by protein aggregation or protein misfolding. In embodiments, amyloid-based disease or condition is any disease or condition that is associated with the increased or decreased presence of amyloid or amyloid like proteins or proteins, such as the presence of amyloid plaques. In embodiments, the amyloid based disease or condition is a neuronal disease or condition, for example, neurodegenerative diseases, in which amyloid-beta peptides, oligomers, fibrils, or plaques are implicated. Non limiting examples of amyloid-based neurodegenerative diseases include Alzheimer's disease, Parkinson's disease, Huntington's disease, Down's Syndrome, and spongiform encephalopathies such as, for example, bovine spongiform encephalopathy (mad cow disease), kuru, Creutzfeldt-Jakob disease, and fatal familial insomnia. In embodiments, other amyloid based diseases that are detected, treated or prevented by the methods of the disclosure include reactive systemic amyloidosis, senile systemic amyloidosis (SAA), familial amyloid polyneuropathy (FAP), familial amyloid cardiomyopathy (FAC), prion disease, coronary heart disease, atherosclerosis, cerebral hemorrhage, AL amyloidosis, type 2 diabetes, diseases or conditions characterized by a loss of cognitive memory capacity such as, for example, mild cognitive impairment (MCI), Lewy body dementia (LBD), hereditary cerebral hemorrhage with amyloidosis (Dutch type) and the Guam Parkinson-Dementia complex. Other diseases which are based on or associated with amyloid-like proteins are progressive supranuclear palsy, multiple sclerosis, HIV-related dementia, ALS (amyotropic lateral sclerosis), inclusion-body myositis (IBM), Adult Onset Diabetes; endocrine tumors, and other diseases, including amyloid-associated ocular diseases that target different tissues of the eye, such as the visual cortex, including cortical visual deficits; the anterior chamber and the optic nerve, including glaucoma; the lens, including cataract due to beta-amyloid deposition; the vitreous, including ocular amyloidosis; the retina, including primary retinal degenerations and macular degeneration, in particular age-related macular degeneration; the optic nerve, including optic nerve drusen, optic neuropathy and optic neuritis; and the cornea, including lattice dystrophy.

In an aspect is provided a method of treating an amyloid-based disease in a subject in need thereof, the method including administering to the subject a therapeutically effective amount of a compound described herein, or a pharmaceutically acceptable salt thereof. In embodiments, the amyloid-based disease is Alzheimer's disease (AD), Parkinson's disease, Huntington's disease, amyotrophic lateral sclerosis (ALS), Lewy body dementia (LBD), or Down's syndrome. In embodiments, the amyloid-based disease is Alzheimer's disease (AD).

In embodiments, the amyloid-based disease is Parkinson's disease. In embodiments, the amyloid-based disease is Huntington's disease. In embodiments, the amyloid-based disease is amyotrophic lateral sclerosis (ALS). In embodiments, the amyloid-based disease is Lewy body dementia (LBD). In embodiments, the amyloid-based disease is Down's syndrome. In embodiments, the amyloid-based disease is a spongiform encephalopathy. In embodiments, the amyloid-based disease is bovine spongiform encephalopathy (mad cow disease). In embodiments, the amyloid-based disease is kuru. In embodiments, the amyloid-based disease is Creutzfeldt-Jakob disease. In embodiments, the amyloid-based disease is fatal familial insomnia. In embodiments, the amyloid-based disease is reactive systemic amyloidosis. In embodiments, the amyloid-based disease is senile systemic amyloidosis (SAA). In embodiments, the amyloid-based disease is familial amyloid polyneuropathy (FAP). In embodiments, the amyloid-based disease is familial amyloid cardiomyopathy (FAC). In embodiments, the amyloid-based disease is prion disease. In embodiments, the amyloid-based disease is coronary heart disease. In embodiments, the amyloid-based disease is atherosclerosis. In embodiments, the amyloid-based disease is cerebral hemorrhage. In embodiments, the amyloid-based disease is AL amyloidosis. In embodiments, the amyloid-based disease is type 2 diabetes. In embodiments, the amyloid-based disease is a disease or conditions characterized by a loss of cognitive memory capacity. In embodiments, the amyloid-based disease is mild cognitive impairment (MCI). In embodiments, the amyloid-based disease is Lewy body dementia (LBD). In embodiments, the amyloid-based disease is hereditary cerebral hemorrhage with amyloidosis (Dutch type). In embodiments, the amyloid-based disease is Guam Parkinson-Dementia complex. In embodiments, the amyloid-based disease is progressive supranuclear palsy. In embodiments, the amyloid-based disease is multiple sclerosis. In embodiments, the amyloid-based disease is HIV-related dementia.

In embodiments, the amyloid-based disease is ALS (amyotropic lateral sclerosis). In embodiments, the amyloid-based disease is inclusion-body myositis (IBM). In embodiments, the amyloid-based disease is Adult Onset Diabetes. In embodiments, the amyloid-based disease is endocrine tumors. In embodiments, the amyloid-based disease is an amyloid-associated ocular disease. In embodiments, the amyloid-based disease is glaucoma. In embodiments, the amyloid-based disease is cataract (e.g., due to beta-amyloid deposition). In embodiments, the amyloid-based disease is ocular amyloidosis. In embodiments, the amyloid-based disease is primary retinal degeneration. In embodiments, the amyloid-based disease is macular degeneration. In embodiments, the amyloid-based disease is age-related macular degeneration. In embodiments, the amyloid-based disease is optic nerve drusen. In embodiments, the amyloid-based disease is optic neuropathy. In embodiments, the amyloid-based disease is optic neuritis. In embodiments, the amyloid-based disease is lattice dystrophy.

In embodiments, the compounds of the present disclosure can be employed for the treatment of Alzheimer's disease, Alzheimer's disease (AD), Parkinson's disease, Huntington's disease, amyotrophic, lateral sclerosis (ALS), Lewy body dementia (LBD), or Down's syndrome. In embodiments, the compounds of the present disclosure can be employed for the detection, diagnosis, treatment and monitoring of Alzheimer's disease. Or the compounds of the present disclosure can be employed for the detection, diagnosis, treatment and monitoring of Creutzfeldt-Jakob disease (CJD).

In embodiments, amyloid-based diseases or conditions also include ocular diseases associated with pathological abnormalities/changes in the tissues of the visual system, particularly associated with amyloid-beta-related pathological abnormalities/changes in the tissues of the visual system, such as, for example, neuronal degradation. In embodiments, said pathological abnormalities occur in different tissues of the eye, such as the visual cortex leading to cortical visual deficits; the anterior chamber and the optic nerve leading to glaucoma; the lens leading to cataract due to beta-amyloid deposition; the vitreous leading to ocular amyloidosis; the retina leading to primary retinal degeneration and macular degeneration, for example age-related macular degeneration; the optic nerve leading to optic nerve drusen, optic neuropathy and optic neuritis; and the cornea leading to lattice dystrophy.

In embodiments, the amyloid or amyloid like proteins and/or proteins that are detected using the methods of the disclosure include amyloid beta peptides (AD), prion peptide (PrP), alpha-synuclein, IAPP (amylin), huntingtin, calcitonin (ACal), atrial natriuretic factor (AANF), apolipoprotein A1 (ApoAl), serum amyloid A (SAA), medin (AMed), prolactin (APro), transthyretin (ATTR), lysozyme (ALys), beta 2 microglobulin (A02M), gelsolin (AGel), keratoepithelin (Aker), cystatin (ACys), immunoglobulin light chain AL (AL), S-IBM or superoxide dismutase. In embodiments, the amyloid peptide detected by the method of the disclosure is Aβ peptide, prion peptide, alpha-synuclein, or superoxide dismutase.

In embodiments, the subjects for the methods of the instant disclosure are any mammal. In embodiments, the subject is a primate (such as a human), canine, feline, ovine, bovine and the like. In embodiments, biological samples that are used in the diagnosis of an amyloid-associated disease or condition for diagnosing a predisposition to an amyloid-associated disease or condition or for monitoring minimal residual disease in a patient or for predicting responsiveness of a patient to a treatment with a compound or a composition or a mixture according to the disclosure and as described herein before are, for example, fluids such as serum, plasma, saliva, gastric secretions, mucus, cerebrospinal fluid, lymphatic fluid, and the like, or tissue or cell samples obtained from an organism such as neural, brain, cardiac or vascular tissue. For determining the presence or absence of the amyloid or amyloid like protein in a sample any immunoassay known to those of ordinary skill in the art may be used such as, for example, assays which utilize indirect detection methods using secondary reagents for detection, ELISA's and immunoprecipitation and agglutination assays.

### VIII. Embodiments

Embodiment P1. A compound of Formula (I), a compound of Formula (II), a pharmaceutically acceptable salt of the compound of Formula (I), or a pharmaceutically acceptable salt of the compound of Formula (II):

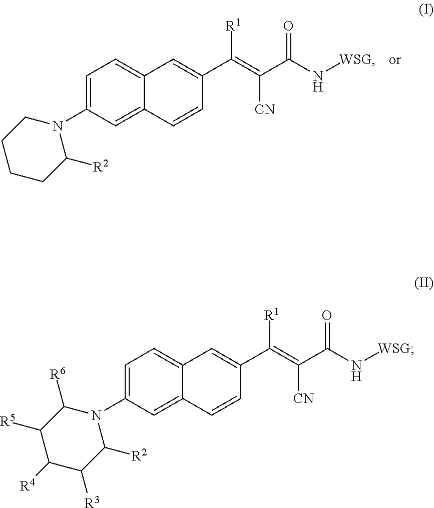

wherein:
WSG is a water soluble group;
R¹, R², R³, R⁴, R⁵, R⁶, and R⁷ are each independently hydrogen, halogen, —CX¹₃, —CHX¹₂, —CH₂X¹, —OCX¹₃, —OCH₂X¹, —OCHX¹₂, —SOₙ₁R^{1A}, —SOᵥ₁NR^{1A}R^{1B}, —CN, —C(O)R^{1A}, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, —NHNR^{1A}R^{1B}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹ is —F, —Cl, —Br, or —I;
n1 is an integer from 0 to 4; and
m1 is 1 or 2; and
v1 is 1 or 2;

Embodiment P2. The compound of embodiment P1, wherein R¹ and R² are each independently substituted or unsubstituted alkyl.

Embodiment P3. The compound of embodiment P1, wherein R¹ and R² are each independently substituted or unsubstituted C₁₋₆ alkyl.

Embodiment P4. The compound of embodiment P1, wherein R¹ and R² are each independently unsubstituted C₁0.6 alkyl.

Embodiment P5. The compound of embodiment P1, wherein R¹ and R² are each independently substituted C₁₋₆ alkyl wherein the substituent is one or more halogen.

Embodiment P6. The compound of embodiment P1 to P5, wherein R³, R⁴, R⁵, R⁶, and R⁷ are hydrogen.

Embodiment P7. The compound of embodiment P1, wherein R¹ is -CL₃, and R², R³, R⁴, R⁵, R⁶, and R⁷ are hydrogen.

Embodiment P8. The compound of embodiment P1, wherein R¹ is —CF₃, and R², R³, R⁴, R⁵, R⁶, and R⁷ are hydrogen.

Embodiment P9. The compound of embodiment P1, wherein R² is —CH₃, and R¹, R³, R⁴, R⁵, R⁶, and R⁷ are hydrogen.

Embodiment P10. The compound of embodiment P1, wherein R² is —CH₂CH₃, and R¹, R³, R⁴, R⁵, R⁶, and R⁷ are hydrogen.

Embodiment P11. The compound of any one of embodiments P1 to P10, wherein WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₃;
each R₃₃ is independently halogen, —OR₃₄, —NR₃₅R₃₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇;
each R₃₄, R₃₅ and R₃₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇;
each R₃₇ is independently halogen, —OR₃₈, —NR₃₉R₄₀, C₁-C₁₀ alkyl, C₁-C₁₀ heteroalkyl, C₁-C₁₀ cycloalkyl, C₁-C₁₀ heterocycloalkyl, —(C₁-C₆alkyl)(C₁-C₁₀heretocycloalkyl), C₁-C₁₀ arylene, or C₁-C₁₀ heteroarylene; and
each of R₃₈, R₃₉ and R₄₀ is independently hydrogen or C₁-C₁₀ alkyl.

Embodiment P12. The compound of any one of embodiments P1 to P10, wherein WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₃;
each R₃₃ is independently halogen, —OR₃₄, —NR₃₅R₃₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇;
each R₃₄, R₃₅ and R³⁶ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇;
each R₃₇ is independently halogen, —OR₃₈, —NR₃₉R₄₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and
each of R₃₈, R₃₉ and R₄₀ is independently hydrogen or C₁-C₁₀ alkyl.

Embodiment P13. The compound of any one of embodiments P1 to P10, wherein WSG is

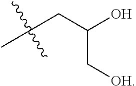 [SMILES: *CC(O)CO]

Embodiment P14. The compound of any one of embodiments P1 to P10, wherein WSG is polyethylene glycol, polypropylene glycol, co-polymer of polyethylene glycol and polypropylene glycol, or alkoxy derivatives thereof.

Embodiment P15. The compound of embodiments P1 to P10, wherein WSG is

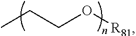

wherein n is an integer from 1-50 and R₈₁ is hydrogen, a C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

Embodiment P16. The compound of embodiment P15, wherein R₈₁ is methyl.

Embodiment P17. The compound of embodiment P15, wherein R₈₁ is CH₂—C═CH.

Embodiment P18. The compound of any one of embodiments P1 to P10, wherein the WSG is

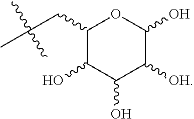 [SMILES: *CC1OC(O)C(O)C(O)C1O]

Embodiment P19. The compound of any one of embodiments P1 to P10, wherein the WSG is

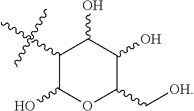

Embodiment P20. The compound of any one of embodiments P1 to P10, wherein WSG is —(C₁-C₁₀ alkyl)-R₃₃—R₃₇, wherein: R³³ is C₁-C₁₀ heteroarylene; and R³⁷ is —(C₁-C₆alkyl)(C₁-C₁₀heretocycloalkyl).

Embodiment P21. The compound of any one of embodiments P1 to P10, wherein WSG is —CH₂OCH₂CH₂OCH₂CH₂OCH₃.

Embodiment P22. A pharmaceutical composition comprising a compound according to any one of embodiments P1 to P20 and a pharmaceutically acceptable excipient.

Embodiment P23. The pharmaceutical composition of embodiment P21, wherein the pharmaceutically acceptable excipient is ethanol, dimethylsulfoxide, polyethylene glycol, polypropylene glycol, aqueous acetate buffer, aqueous citrate buffer, aqueous phosphate buffer, aqueous carbonate buffer, cyclodextrin, corn oil, vitamin E, polysorbate, bile acid, or a combination of two or more thereof.

Embodiment P24. A composition comprising a compound according to any one of embodiments P1 to P20 and an amyloid or amyloid like protein.

Embodiment P25. The composition of embodiment P23, wherein the amyloid or amyloid like protein is Aβ peptide, prion peptide, alpha-synuclein, or superoxide dismutase.

Embodiment P26. A method of detecting an amyloid or amyloid like protein comprising
(a) contacting a compound according to any one of claims 1 to 20 with a sample potentially comprising the amyloid or amyloid like protein, wherein in presence of an amyloid or amyloid like protein the compound forms a detectable complex; and
(b) detecting the formation of the detectable complex such that the presence or absence of the detectable complex correlates with the presence or absence of the amyloid or amyloid like protein.

Embodiment P27. The method of embodiment P25, wherein the detection of the formation of the detectable complex is performed by measuring a signal generated by the detectable complex.

Embodiment P28. The method of embodiment P26, wherein the signal generated by the detectable complex is an electromagnetic signal.

Embodiment P29. The method of embodiment P27, wherein the electromagnetic signal is a fluorescence signal.

Embodiment P30. The method of embodiment P28, wherein the fluorescence signal is measures at a wavelength of 450-650 nm.

Embodiment P31. The method of embodiment P28, wherein the fluorescence signal is measures at a wavelength of 520-540 nm.

Embodiment P32. The method of any one of embodiments P25 to P30, wherein the amyloid or amyloid like protein is Aβ peptide, prion peptide, alpha-synuclein, or superoxide dismutase.

Embodiment P33. The method of any one of embodiments P25 to P30, wherein the amyloid or amyloid like protein is beta amyloid (1-42) (Aβ(1-42)).

Embodiment P34. The method of any one of embodiments P25 to P30, wherein the detection of the formation of the detectable complex is performed within about 1 sec, about 5 sec, about 1 min, about 10 min, about 30 min or about 60 min of the contacting of the compound according to any one of embodiments P1 to P20 with the sample.

Embodiment P35. The method of any one of embodiments P25 to P30, wherein the detection of the formation of the detectable complex is performed within about 1-5 minutes of the contacting of the compound according to any one of embodiments P1 to P20 with the sample.

Embodiment P36. A method of determining the presence or absence of one or more disease or condition in a subject comprising
(a) administering to the subject an effective amount of a compound according to any one of Embodiments P1 to P20 or a pharmaceutical composition thereof, wherein in presence of the disease or condition the administered compound forms a detectable complex; and
(b) detecting the formation of the detectable complex such that presence or absence of detectable complex correlates with the presence or absence of the disease or condition.

Embodiment P37. The method of embodiment P35, wherein the disease is characterized by protein aggregation or protein misfolding.

Embodiment P38. The method of embodiment P35, wherein the disease is an amyloid based disease or condition.

Embodiment P39. The method of embodiment P35, wherein the disease is Alzheimer's disease, Parkinson's disease, Huntington's disease, amyotrophic lateral sclerosis, Lewy body dementia, or Down's syndrome.

### IX. Additional Embodiments

Embodiment 1. A compound having the formula:

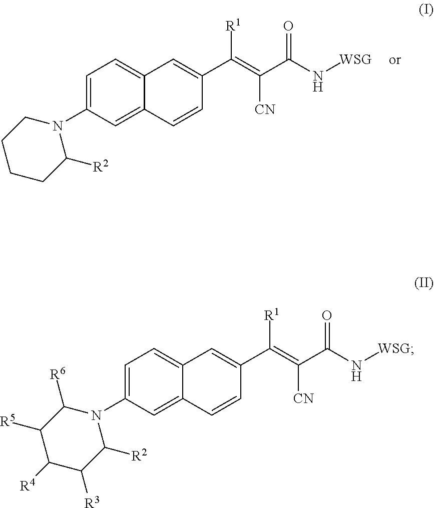

or a pharmaceutically acceptable salt thereof;
wherein:
WSG is a water soluble group;
W¹ is O, N(R¹⁴), or C(R⁴)₂;
R¹⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹, R², R³, R⁴, R⁵, and R⁶ are each independently hydrogen, halogen, —CX¹₃, —CHX¹₂, —CH₂X¹, —OCX¹₃, —OCH₂X¹, —OCHX₂, —SOₙ₁R^{1A}, —SOᵥ₁NR^{1A}R^{1B}, —CN, —C(O)R^{1A}, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, NHNR^{1A}R^{1B}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂J, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹ is —F, —Cl, —Br, or —I;
n1 is independently an integer from 0 to 4;
m1 is independently 1 or 2; and
v1 is independently 1 or 2.

Embodiment 2. The compound of embodiment 1, having the formula:

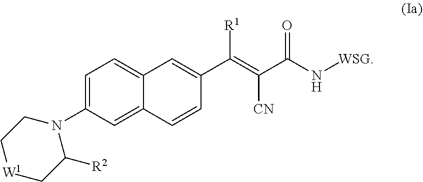

Embodiment 3. The compound of embodiment 1 or embodiment 2, having the formula:

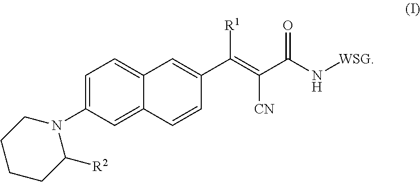

Embodiment 4. The compound of embodiment 2 or embodiment 3, wherein R¹ is hydrogen.

Embodiment 5. The compound of one of embodiments 2 to 4, wherein R² is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

Embodiment 6. The compound of one of embodiments 2 to 4, wherein R² is hydrogen or unsubstituted C₁-C₄ alkyl.

Embodiment 7. The compound of one of embodiments 2 to 4, wherein R² is unsubstituted methyl, unsubstituted ethyl, or unsubstituted propyl.

Embodiment 8. The compound of one of embodiments 2 to 4, wherein R³, R⁴, R⁵, and R⁶ are hydrogen.

Embodiment 9. The compound of embodiment 1, having the formula:

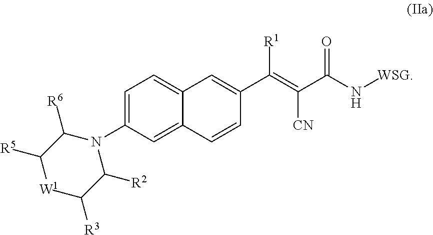

Embodiment 10. The compound of embodiment 1 or embodiment 9, having the formula:

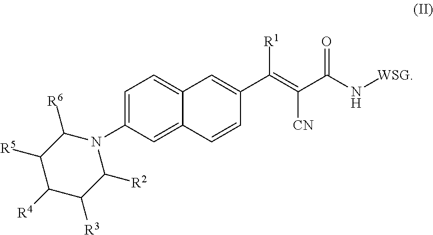

Embodiment 11. The compound of embodiment 9 or embodiment 10, wherein R¹ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

Embodiment 12. The compound of embodiment 9 or embodiment 10, wherein R¹ is —CF₃ or substituted or unsubstituted C₁-C₄ alkyl.

Embodiment 13. The compound of embodiment 9 or embodiment 10, wherein R¹ is —CF₃ or unsubstituted methyl.

Embodiment 14. The compound of one of embodiments 9 to 13, wherein R², R³, R⁴, R, and R⁶ are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OC13, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

Embodiment 15. The compound of one of embodiments 9 to 13, wherein R², R³, R⁴, R, and R⁶ are hydrogen.

Embodiment 16. The compound of one of embodiments 1 to 15, wherein WSG is hydrogen, R³³-substituted or unsubstituted C₁-C₁₀ alkyl, R³³-substituted or unsubstituted 2 to 10 membered heteroalkyl, R³³-substituted or unsubstituted C₃-C₁₀ cycloalkyl, R³³-substituted or unsubstituted 3 to 10 membered heterocycloalkyl, R³³-substituted or unsubstituted C₆-C₁₀ aryl, or R³³-substituted or unsubstituted 5 to 10 membered heteroaryl;
R³³ is independently halogen, —OR³⁴, —NR³⁵R³⁶, R³⁷-substituted or unsubstituted C₁-C₁₀ alkyl, R³⁷-substituted or unsubstituted 2 to 10 membered heteroalkyl, R³⁷-substituted or unsubstituted C₃-C₁₀ cycloalkyl, R³⁷-substituted or unsubstituted 3 to 10 membered heterocycloalkyl, R³⁷-substituted or unsubstituted C₆-C₁₀ aryl, or R³⁷-substituted or unsubstituted 5 to 10 membered heteroaryl;
R³⁴, R⁵, and R³⁶ are independently hydrogen, R³⁷-substituted or unsubstituted C₁-C₁₀ alkyl, R³⁷-substituted or unsubstituted 2 to 10 membered heteroalkyl, R³⁷-substituted or unsubstituted C₃-C₁₀ cycloalkyl, R³⁷-substituted or unsubstituted 3 to 10 membered heterocycloalkyl, R³⁷-substituted or unsubstituted C₆-C₁₀ aryl, or R³⁷-substituted or unsubstituted 5 to 10 membered heteroaryl;
R³⁷ is independently halogen, —OR³⁸, —NR³⁹R⁴⁰, unsubstituted C₁-C₁₀ alkyl, unsubstituted 2 to 10 membered heteroalkyl, unsubstituted C₃-C₁₀ cycloalkyl, unsubstituted 3 to 10 membered heterocycloalkyl, -(unsubstituted C₁-C₆ alkyl)-(unsubstituted 3 to 10 membered heterocycloalkyl), unsubstituted C₆-C₁₀ aryl, or unsubstituted 5 to 10 membered heteroaryl; and R³⁸, R³⁹, and R⁴⁰ are independently hydrogen or unsubstituted C₁-C₁₀ alkyl.

Embodiment 17. The compound of one of embodiments 1 to 15, wherein WSG is

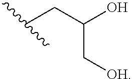 [SMILES: *CC(O)CO]

Embodiment 18. The compound of one of embodiments 1 to 15, wherein WSG is polyethylene glycol, polypropylene glycol, co-polymer of polyethylene glycol and polypropylene glycol, or alkoxy derivatives thereof.

Embodiment 19. The compound of one of embodiments 1 to 15, wherein WSG is

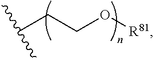

wherein n is an integer from 1 to 50 and R⁸¹ is hydrogen, substituted or unsubstituted C₁-C₁₀ alkyl, substituted or unsubstituted C₂-C₁₀ alkenyl, or substituted or unsubstituted C₂-C₁₀ alkynyl.

Embodiment 20. The compound of embodiment 19, wherein R⁸¹ is unsubstituted methyl.

Embodiment 21. The compound of embodiment 19, wherein R⁸¹ is —CH₂—C≡CH.

Embodiment 22. The compound of one of embodiments 1 to 15, wherein WSG is

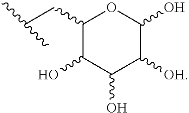 [SMILES: *CC1OC(O)C(O)C(O)C1O]

Embodiment 23. The compound of one of embodiments 1 to 15, wherein WSG is

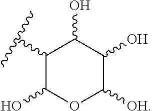

Embodiment 24. The compound of one of embodiments 1 to 15, wherein WSG is -(unsubstituted C₁-C₁₀ alkyl)-R³³—R³⁷, wherein
R³³ is unsubstituted 5 to 10 membered heteroarylene; and
R³⁷ is -(unsubstituted C₁-C₆ alkyl)-(unsubstituted 5 to 10 membered heterocycloalkyl).

Embodiment 25. The compound of one of embodiments 1 to 15, wherein WSG is

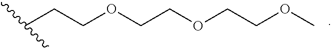 [SMILES: *CCOCCOCCOC]

Embodiment 26. The compound of one of embodiments 1 to 15, wherein WSG is —CH₂OCH₂CH₂OCH₂CH₂OCH₃.

Embodiment 27. A pharmaceutical composition comprising a compound of one of embodiments 1 to 26, and a pharmaceutically acceptable excipient.

Embodiment 28. The pharmaceutical composition of embodiment 27, wherein the pharmaceutically acceptable excipient is ethanol, dimethylsulfoxide, polyethylene glycol, polypropylene glycol, aqueous acetate buffer, aqueous citrate buffer, aqueous phosphate buffer, aqueous carbonate buffer, cyclodextrin, corn oil, vitamin E, polysorbate, bile acid, or a combination of two or more thereof.

Embodiment 29. A composition comprising a compound of one of embodiments 1 to 26, and an amyloid or amyloid like protein.

Embodiment 30. The composition of embodiment 29, wherein the amyloid or amyloid like protein is Aβ peptide, prion peptide, alpha-synuclein, superoxide dismutase, tau, phosphorylated tau, TDP-43, or TMEM106b.

Embodiment 31. A method of detecting an amyloid or amyloid like protein comprising
(a) contacting a compound of one of embodiments 1 to 26, with a sample potentially comprising the amyloid or amyloid like protein, wherein in presence of an amyloid or amyloid like protein the compound forms a detectable complex; and
(b) detecting the formation of the detectable complex such that the presence or absence of the detectable complex correlates with the presence or absence of the amyloid or amyloid like protein.

Embodiment 32. The method of embodiment 31, wherein the detection of the formation of the detectable complex is performed by measuring a signal generated by the detectable complex.

Embodiment 33. The method of embodiment 32, wherein the signal generated by the detectable complex is an electromagnetic signal.

Embodiment 34. The method of embodiment 33, wherein the electromagnetic signal is a fluorescence signal.

Embodiment 35. The method of embodiment 34, wherein the fluorescence signal is measured at a wavelength of from 450 nm to 650 nm.

Embodiment 36. The method of embodiment 34, wherein the fluorescence signal is measured at a wavelength of from 520 nm to 540 nm.

Embodiment 37. The method of one of embodiments 31 to 36, wherein the amyloid or amyloid like protein is Aβ peptide, prion peptide, alpha-synuclein, superoxide dismutase, tau, phosphorylated tau, TDP-43, or TMEM106b.

Embodiment 38. The method of one of embodiments 31 to 36, wherein the amyloid or amyloid like protein is beta amyloid (1-42) (Aβ (1-42)).

Embodiment 39. The method of one of embodiments 31 to 36, wherein the detection of the formation of the detectable complex is performed within about 1 sec, about 5 sec, about 1 min, about 10 min, about 30 min or about 60 min of the contacting of the compound with the sample.

Embodiment 40. The method of one of embodiments 31 to 36, wherein the detection of the formation of the detectable complex is performed within about 1-5 minutes of the contacting of the compound with the sample.

Embodiment 41. A method of determining the presence or absence of one or more disease or condition in a subject comprising
(a) administering to the subject an effective amount of a compound of one of embodiments 1 to 26, or a pharmaceutical composition thereof, wherein in presence of the disease or condition the administered compound forms a detectable complex; and
(b) detecting the formation of the detectable complex such that presence or absence of detectable complex correlates with the presence or absence of the disease or condition.

Embodiment 42. The method of embodiment 41, wherein the disease is characterized by protein aggregation or protein misfolding.

Embodiment 43. The method of embodiment 41, wherein the disease is an amyloid based disease or condition.

Embodiment 44. The method of embodiment 41, wherein the disease is Alzheimer's disease, Parkinson's disease, Huntington's disease, amyotrophic lateral sclerosis, Lewy body dementia, or Down's syndrome.

### EXAMPLES

### Example 1: Exploring the Effect of Aliphatic Substituents on Aryl Cyano Amides on Enhancement of Fluorescence Upon Binding to Amyloid-β Aggregates

Alzheimer's Disease (AD) is a neurodegenerative disease associated with cognitive decline and dementia and is the 6th leading cause of death in the United States.^{1,2} Pathologically, AD is characterized by the accumulation and deposition of aggregated Amyloid Beta (AD) peptides that purportedly cause damage to healthy neurons in the brain.^{3,4} Current methods for detection of amyloids comprised of Aβ or other amyloidogenic proteins include the use of small molecule probes that target these aggregates.^{5,6} For instance, Positron Emission Tomography (PET) agents are currently used to aid in clinical diagnosis of AD, but their utility outside of clinical trials has been limited by the short half-life of the radioligands and high cost associated with PET scans.^{7,8} Fluorescent amyloid-binding agents, on the other hand, have emerged as a viable alternative approach to aiding in the diagnosis of amyloid-associated neurodegenerative diseases⁹⁻¹¹ that have many advantages over PET agents including: 1) high spatial resolution, 2) low cost, and 3) the ability to spectroscopically discriminate between amyloid species of different disease origin.^{6,12-14}

Historically, fluorescent probes such as Thioflavin T (ThT) and Congo Red (CR) have been used for detection of Aβ plaques in post-mortem brain samples,¹⁵ but these probes have not been used for in vivo ante-mortem detection of amyloidosis, presumably due, in part, to their poor biocompatibility properties.¹⁶ ThT and CR are part of a large class of fluorescent compounds called molecular rotors, which consist of electron-rich donor (D) units in conjugation through a π scaffold to electron-poor acceptors (A) (otherwise known as the D-π-A motif).¹⁵⁻¹⁷ Upon photoexcitation, molecular rotors either form a fluorescent locally excited (LE) state or a Twisted Intramolecular Charge-Transfer (TICT) complex.¹⁷ When in the LE state, photoexcited molecular rotors can relax to the ground state and release energy through emission of a photon. However, while in the TICT state, molecular rotors typically release energy through internal non-radiative modes.¹⁸

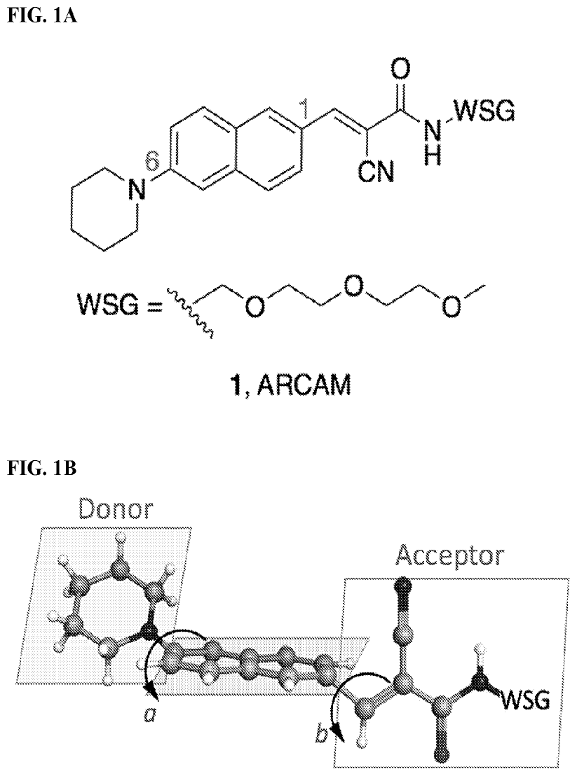

The fluorescence properties of molecular rotors can be significantly influenced by the surrounding microenvironment.^{14,19-22} For instance, ARyl Cyano AMide 1 (ARCAM, FIG. 1A) exhibits enhanced fluorescence intensity upon binding to Aβ aggregates in solution and in tissue compared to free probe in solution.¹¹⁻²³ The binding of ARCAM to an amyloid binding pocket can restrict rotation of specific bonds (e.g., bonds a and b in FIG. 1B) between the D and A groups, leading to a higher fraction of molecules in the LE state versus the TICT state and resulting in a higher overall emission intensity compared to unbound molecules. This enhanced fluorescence property of ARCAM when bound to amyloids has recently been shown to enable and facilitate ante-mortem detection of Aβ-containing deposits in the retinas of a mouse model for AD.²³

In order to develop molecular rotors with improved fluorescence contrast when bound to amyloid versus free in solution, small aliphatic substituents on the vinyl group or on the 2-position of the piperidine of ARCAM (compounds 2-5, FIG. 1C) were systematically introduced to explore whether these modifications could affect the fluorescence intensity of the probe when bound versus unbound to Aβ aggregates. These substituents could sterically impede rotation of bonds a or b between the naphthalene and the donor piperidine or acceptor cyano groups in ARCAM. Introducing these steric interactions could, in turn, disfavor the formation of the LE state (or promote the formation of the TICT state) and reduce the overall background fluorescence intensity of the free molecule in solution. Binding of these rotationally restricted analogs of ARCAM (compounds 2-5) to amyloid aggregates could help drive planarization of the molecules and result in an increased ratio of the LE versus TICT photoexcited state, leading to improved enhancement of fluorescence intensity of the amyloid-bound compounds compared to the parent ARCAM compound. The effects of aliphatic substituents on the quantum yield (QY) of ARCAM analogs 2-5 were compared to fluorescence enhancement of these probes bound to Aβ aggregates in solution. Whether electronic effects of aliphatic substituents could influence the excitation or emission profile of these ARCAM analogs were investigated. Only certain aliphatic substituents on the ARCAM scaffold led to an improvement of fluorescence enhancement when bound versus not bound to amyloid, which represents an important step towards developing new design principles for generating high contrast fluorescent probes for detecting amyloids associated with neurodegenerative diseases.

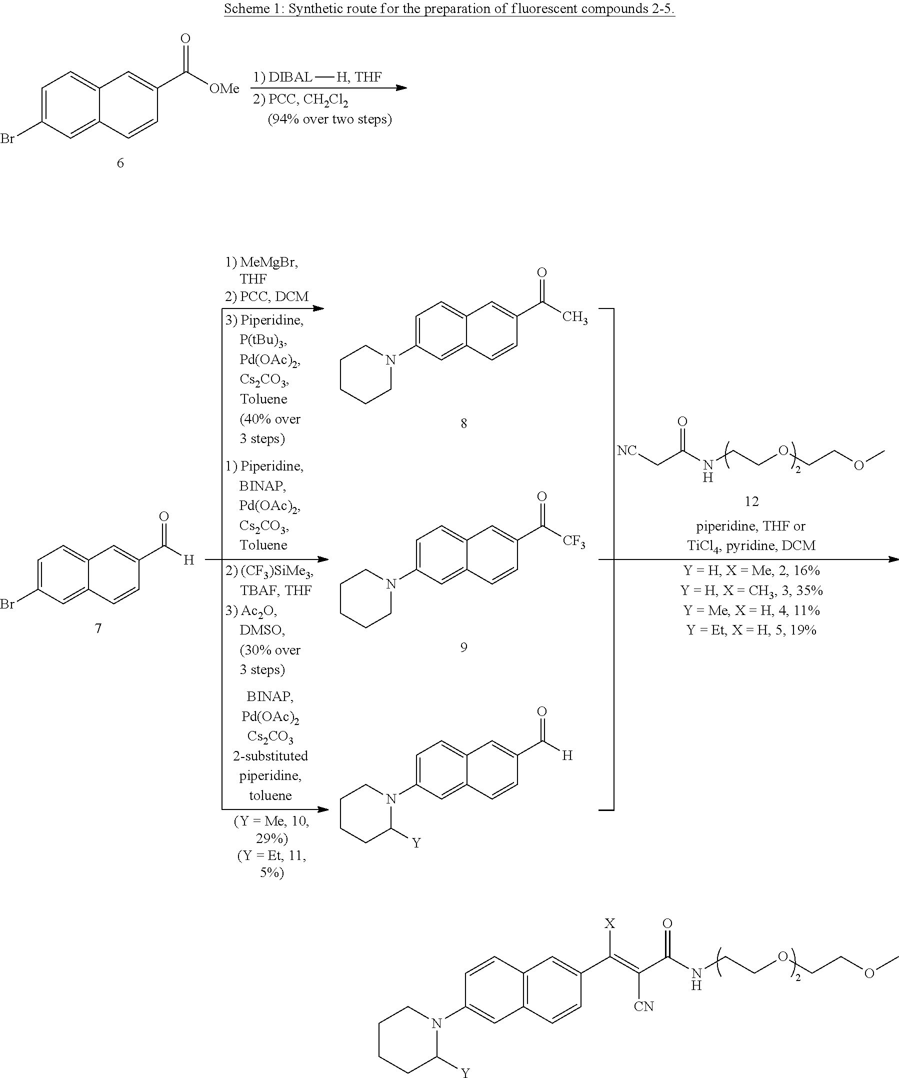

### Example 2: Experimental Procedures and Characterization Data

### General Synthetic Procedures

### Buchwald-Hartwig Coupling:

To a solution of Pd(OAc)₂ (0.1 eq) in degassed anhydrous toluene (0.026 M) in a microwave vial, rac-BINAP (0.1 eq) was added. The solution was degassed and stirred for 20 minutes at room temperature. After, 6-bromo-2-naphthaldehyde (1 eq), Cs₂CO₃ (1.34 eq), and substituted piperidine (1.2 eq) were added. The solution was degassed, the vial sealed, and the mixture was left stirring overnight at reflux. Upon completion, DCM was added, and the mixture was filtered through a pad of celite and purified via silica column chromatography.

### Knoevenagel Condensation:

To a solution of aryl aldehyde (1 eq) in anhydrous THE (0.29 M) in a microwave tube was added 2-cyano-N-(2-(2-(2-methoxyethoxy)ethoxy)ethyl)acetamide (1.2 eq) and piperidine (0.01 eq). The tube was sealed, heated to 50° C. under inert atmosphere, and left stirring overnight. Upon completion, the reaction mixture was cooled to room temperature, concentrated under reduced pressure, and purified via silica column chromatography.

### Titanium-Catalyzed Knoevenagel Condensation:

To a solution of ketone (1 eq) and 2-cyano-N-(2-(2-(2-methoxyethoxy)ethoxy)ethyl)acetamide (1.2 eq) in anhydrous DCM (0.0725 M) at 0° C. was added TiCl₄ (1.0 M in DCM, 2 eq). Molecular sieves (4 A) was added and the mixture was stirred for 30 minutes. Pyridine (1.1 eq) was added dropwise and the mixture was stirred for 1 hour. The remaining pyridine (2.2 eq) was added dropwise. The mixture was allowed to warm to room temperature and stirred overnight. Upon completion, the reaction mixture was filtered through celite. The reaction was quenched with 3M HCl (0.27 M) and extracted with DCM. After, it was dried with sodium sulfate, filtered, concentrated under reduced pressure, and purified via silica column chromatography.

### Synthesis of Naphthyl Aldehyde 7

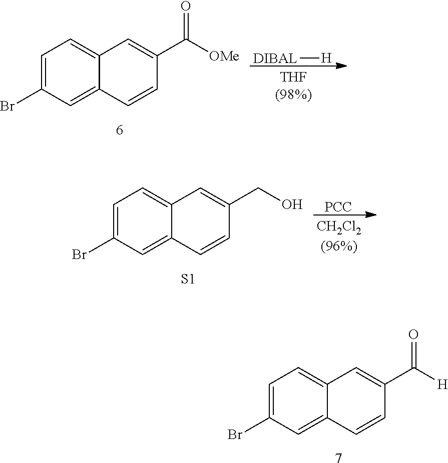

### (6-bromonaphthalen-2-yl)methanol (S1)

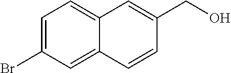 [SMILES: OCc1ccc2cc(Br)ccc2c1]

To a solution of methyl 6-bromo-2-naphthoate (6) (2.6 g, 10 mmol) in anhydrous THE (30 mL) at 0° C. under inert atmosphere was added diisobutylaluminium hydride (1.0 M in heptane, 30 mL, 30 mmol) dropwise. The reaction mixture was stirred and allowed to reach room temperature overnight. Upon completion, the reaction mixture was quenched with isopropyl alcohol, methanol, and saturated sodium potassium tartrate solution. The THE was concentrated under reduced pressure, the new mixture dissolved in DCM and extracted, retaining the organic layer. After, it was dried with sodium sulfate, filtered, and concentrated under reduced pressure. (6-bromonaphthalen-2-yl) methanol (S1) (2.3 g, 98%) was obtained without purification. ¹H NMR (Nuclear Magnetic Resonance) data matched previously reported data²¹.

### 6-bromo-2-naphthaldehyde (7)

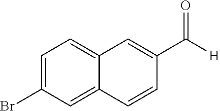 [SMILES: O=Cc1ccc2cc(Br)ccc2c1]

To a solution of (6-bromonaphthalen-2-yl)methanol (S1) (1.5 g, 6.3 mmol) in Dichloromethane (DCM) (60 mL) at 0° C. was added pyridininum chlorochromate (1.5 g, 6.9 mmol). The reaction mixture was stirred at room temperature overnight. Upon completion, diethyl ether was added, and the resulting mixture was filtered through a pad of silica and concentrated under reduced pressure. 6-bromo-2-naphthaldehyde (7) (1.4 g, 96%) was obtained without purification. ¹H NMR data matched previously reported data²′.

### Synthetic Procedure for Probes 2 and 3

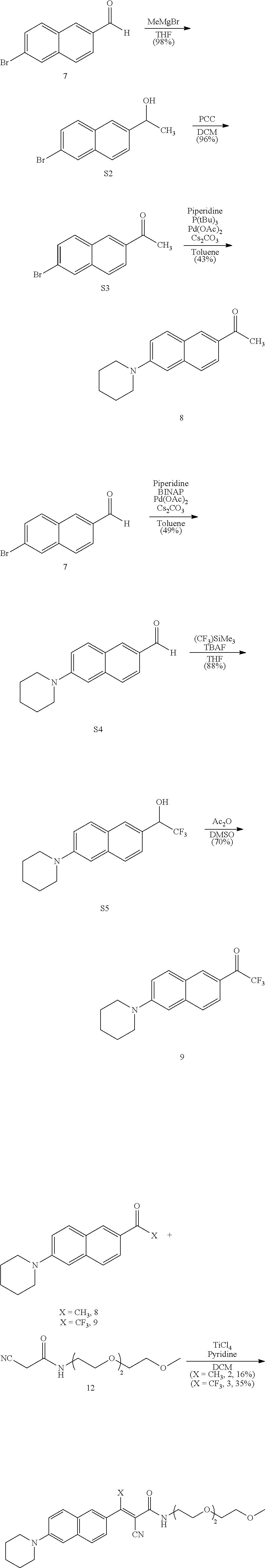

### 1-(6-bromonaphthalen-2-yl)ethan-1-ol (S2)

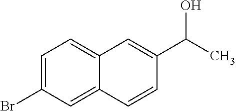

To a solution of 6-bromo-2-naphthaldehyde (7) (500 mg, 2.1 mmol) in anhydrous THF (24 mL) at 0° C. under inert atmosphere was added methylmagnesium bromide (3.0 M in ether, 0.85 mL, 2.5 mmol) dropwise. The reaction mixture was stirred and allowed to reach room temperature overnight. Upon completion, it was quenched with ammonium chloride, followed by an extraction with EtOAc. The organic layer was dried with sodium sulfate, filtered, and concentrated under reduced pressure. Alcohol 1-(6-bromonaphthalen-2-yl)ethan-1-ol (S2) (515 mg, 98%) was obtained as a white solid.

Rf=0.29 (hexane/EtOAc 80:20); ¹H NMR (400 MHz, Chloroform-d) δ 7.99 (s, 1H), 7.81-7.67 (m, 3H), 7.54 (td, J=8.0, 7.1, 1.9 Hz, 2H), 5.06 (q, J=6.5 Hz, 1H), 1.75 (s, 1H), 1.58 (d, J=6.5 Hz, 3H); ¹³C NMR (101 MHz, CDCl₃) δ 143.78, 133.97, 131.78, 129.87, 129.71, 127.53, 124.93, 123.89, 123.72, 119.76, 70.48, 25.23; ESI-MS: 233.13 [M−H2O]⁺.

### 1-(6-bromonaphthalen-2-yl)ethan-1-one (S3)

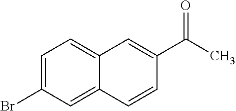

To a solution of 1-(6-bromonaphthalen-2-yl)ethan-1-ol (S2) (151 mg, 0.6 mmol) in DCM (10 mL) at 0° C. was added pyridininum chlorochromate (142 mg, 0.66 mmol). The reaction mixture was stirred at room temperature overnight. Upon completion, diethyl ether was added and the resulting mixture was filtered through a pad of silica and concentrated under reduced pressure. 1-(6-bromonaphthalen-2-yl)ethan-1-one (compound S3) (143 mg, 96%) was obtained without purification as a white solid.

Rf=0.52 (hexane/EtOAc 80:20); ¹H NMR (400 MHz, Chloroform-d) δ 8.34 (s, 1H), 8.02-7.91 (m, 2H), 7.72 (dd, J=13.4, 8.5 Hz, 2H), 7.55 (d, J=6.2 Hz, 1H), 2.67 (s, 3H); ¹³C NMR (101 MHz, CDCl₃) δ 197.66, 136.38, 134.70, 131.11, 130.88, 129.97, 127.56, 127.38, 125.10, 124.94, 122.83, 26.76; ESI-MS: 251.15 [M+H]⁺.

### 1-(6-(piperidin-1-yl)naphthalen-2-yl)ethan-1-one (8)

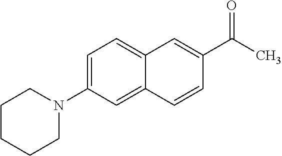 [SMILES: CC(=O)c1ccc2cc(N3CCCCC3)ccc2c1]

To a solution of Pd(OAc)₂ (28 mg, 0.12 mmol) in degassed anhydrous toluene (4.5 mL) in a microwave vial, P(tBu)₃ (0.11 mL, 0.45 mmol) was added. The solution was degassed and stirred for 20 minutes at room temperature. After, 1-(6-bromonaphthalen-2-yl)ethan-1-one (S3), (295 mg, 1.2 mmol), Cs₂CO₃ (521 mg, 1.6 mmol), and piperidine (0.14 mL, 1.42 mmol) were added. The solution was degassed, the vial sealed, and the mixture was left stirring overnight at reflux. Upon completion, DCM was added and the mixture was filtered through a pad of celite and purified via silica column chromatography using hexane/EtOAc (88:12) as the eluent. 1-(6-(piperidin-1-yl)naphthalen-2-yl)ethan-1-one (compound 8) was obtained as a yellow solid (73 mg, 24%).

Rf=0.31 (hexane/EtOAc 90:10); ¹H NMR (400 MHz, Chloroform-d) δ 8.33 (s, 1H), 7.97 (d, J=8.7 Hz, 1H), 7.80 (d, J=9.1 Hz, 1H), 7.67 (d, J=8.7 Hz, 1H), 7.32 (dd, J=9.1, 2.5 Hz, 1H), 7.09 (s, 1H), 3.39-3.32 (m, 4H), 2.69 (s, 3H), 1.81-1.73 (m, 4H), 1.72-1.62 (m, 2H); ¹³C NMR (126 MHz, cdcl₃) δ 197.84, 151.63, 137.52, 131.58, 130.41, 130.08, 126.66, 126.37, 124.44, 119.69, 108.85, 49.85, 26.51, 25.63, 24.35; ESI-MS: 254.26 [M+H]⁺.

### (E)-2-cyano-N-(2-(2-(2-methoxyethoxy)ethoxy)ethyl)-3-(6-(piperidin-1-yl)naphthalen-2-yl)but-2-enamide (2)

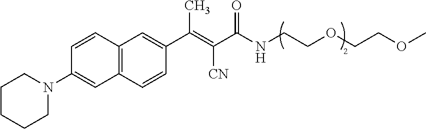

Compound such as (E)-2-cyano-N-(2-(2-(2-methoxyethoxy)ethoxy)ethyl)-3-(6-(piperidin-1-yl)naphthalen-2-yl)but-2-enamide (2) was synthesized from 1-(6-(piperidin-1-yl)naphthalen-2-yl)ethan-1-one (8) (73 mg, 0.29 mmol) and α-cyanoamide (12)²¹ (80 mg, 0.35 mmol) according to the general procedure for Titanium-Catalyzed Knoevenagel Condensation. (E)-2-cyano-N-(2-(2-(2-methoxyethoxy)ethoxy)ethyl)-3-(6-(piperidin-1-yl)naphthalen-2-yl)but-2-enamide (2) (11 mg, 16%) was obtained as a yellow solid after purification by column chromatography on silica gel using hexane/EtOAc (90:10) as the eluent.

Rf=0.52 (100% EtOAc); ¹H NMR (400 MHz, Methylene Chloride-d₂) δ 7.80 (d, J=2.0 Hz, 1H), 7.76-7.59 (m, 2H), 7.42 (dd, J=8.6, 2.0 Hz, 1H), 7.31 (dd, J=9.1, 2.4 Hz, 1H), 7.09 (d, J=2.5 Hz, 1H), 6.80 (s, 1H), 3.66-3.41 (m, 11H), 3.33-3.18 (m, 8H), 2.62 (s, 3H), 1.73-1.67 (m, 4H), 1.67-1.56 (m, 2H); ¹³C NMR (126 MHz, cd₂cl₂) δ 167.38, 162.57, 151.66, 136.04, 134.81, 129.66, 127.51, 127.33, 127.21, 125.28, 120.74, 118.44, 109.53, 106.79, 72.39, 70.94, 70.86, 69.88, 59.14, 50.65, 40.30, 30.21, 26.22, 24.87, 22.88; HRMS 466.2700 calcd for [C₂₇ H₃₆ N₃ O₄]⁺, found 466.2669.

Upon synthesis and purification of (E)-2-cyano-N-(2-(2-(2-methoxyethoxy)ethoxy)ethyl)-3-(6-(piperidin-1-yl)naphthalen-2-yl)but-2-enamide (2), a mixture of inseparable E and Z isomers were made, with an estimated 8.5:1 ratio of E:Z by NMR and assigned according to literature precedence³⁰. This issue with isomer mixture formation was observed previously with the synthesis of ARCAM, and does not appear to significantly affect the fluorescence and binding to amyloids²¹⁻²².

### 6-(piperidin-1-yl)-2-naphthaldehyde (S4)

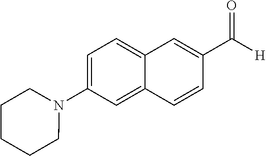 [SMILES: O=Cc1ccc2cc(N3CCCCC3)ccc2c1]

Aldehyde such as 6-(piperidin-1-yl)-2-naphthaldehyde (S4) was synthesized from 6-bromo-2-naphthaldehyde (7) (1 g, 4.25 mmol) and piperidine (0.5 mL, 5.1 mmol) according to the general procedure for Buchwald-Hartwig Coupling (see above). 6-(piperidin-1-yl)-2-naphthaldehyde (S4) (500 mg, 49%) was obtained as a yellow solid after purification by column chromatography on silica gel using hexane/EtOAc (90:10) as the eluent. ¹H NMR data matched previously reported data²¹.

### 2,2,2-trifluoro-1-(6-(piperidin-1-yl)naphthalen-2-yl)ethan-1-ol (S5)

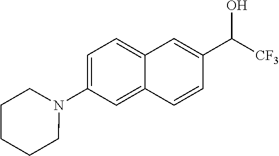 [SMILES: OC(c1ccc2cc(N3CCCCC3)ccc2c1)C(F)(F)F]

To a solution of 6-(piperidin-1-yl)-2-naphthaldehyde (S4) (318 mg, 1.33 mmol) in anhydrous THF (2 mL) at 0° C. was added (Trifluoromethyl)trimethylsilane (0.25 mL, 1.73 mmol). Tetrabutylammonium fluoride (1.0 M in THF, 0.01 mL, 0.001 mmol) was added dropwise and allowed to reach room temperature overnight. The reaction was cooled back down to 0° C. and tetrabutylammonium fluoride (1.0 M in THF, 0.13 mL, 0.13 mmol) and H₂O (0.2 mL) was added. The reaction mixture was allowed to stir for 1.5 hour at room temperature. Upon completion, the reaction was extracted with EtOAc, dried with sodium sulfate, filtered, and concentrated under reduced pressure. 2,2,2-trifluoro-1-(6-(piperidin-1-yl)naphthalen-2-yl)ethan-1-ol (compound S5) was obtained as a colorless oil after silica column chromatography with hexane/EtOAc (70:30) as the eluent (364 mg, 88%).

Rf=0.36 (hexane/EtOAc 80:20); ¹H NMR (500 MHz, Chloroform-d) δ 7.78 (s, 1H), 7.70 (dd, J=8.7, 5.5 Hz, 2H), 7.46 (d, J=8.5 Hz, 1H), 7.31 (d, J=9.0 Hz, 1H), 7.11 (s, 1H), 5.12 (q, J=6.8 Hz, 1H), 3.28 (t, J=5.4 Hz, 4H), 2.69 (s, 1H), 1.76 (p, J=5.7 Hz, 4H), 1.63 (p, J=6.0 Hz, 2H); ¹³C NMR (126 MHz, cdcl₃) δ 153.81, 150.85, 135.33, 128.98, 127.33, 127.04, 125.69, 124.76, 123.45, 120.67, 109.96, 73.37, 50.85, 25.86, 24.45; ESI-MS: 310.38 [M+H]⁺.

### 2,2,2-trifluoro-1-(6-(piperidin-1-yl)naphthalen-2-yl)ethan-1-one (9)

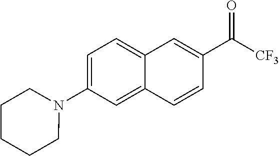 [SMILES: O=C(c1ccc2cc(N3CCCCC3)ccc2c1)C(F)(F)F]

A solution of acetic anhydride (109 μL, 1.16 mmol) in Dimethyl Sulfoxide DMSO (2 mL) was stirred at room temperature for 10 minutes. 2,2,2-trifluoro-1-(6-(piperidin-1-yl)naphthalen-2-yl)ethan-1-ol (S5) (90 mg, 0.29 mmol) in DMSO (0.5 mL) was added and allowed to stir at room temperature overnight. Upon completion, 10% NaHCO₃ (12.5 mL) was added and stirred for one hour. The reaction mixture was then extracted with EtOAc, dried with sodium sulfate, filtered, and concentrated under reduced pressure. 2,2,2-trifluoro-1-(6-(piperidin-1-yl)naphthalen-2-yl)ethan-1-one (compound 9) was obtained as a yellow solid after silica column chromatography with hexane/EtOAc (96:4) as the eluent (62 mg, 70%).

Rf=0.76 (hexane/EtOAc 80:20); ¹H NMR (400 MHz, Chloroform-d) δ 8.43 (s, 1H), 7.95 (d, J=8.7 Hz, 1H), 7.80 (d, J=9.1 Hz, 1H), 7.65 (d, J=8.7 Hz, 1H), 7.31 (dd, J=9.2, 2.5 Hz, 1H), 7.04 (s, 1H), 3.45-3.38 (m, 4H), 1.79-1.64 (m, 6H); ¹³C NMR (126 MHz, cdcl₃) δ 179.77, 154.17, 138.88, 133.23, 131.53, 127.18, 125.16, 119.47, 118.44, 116.12, 108.20, 49.45, 29.85, 25.58, 24.44; ESI-MS: 308.35 [M+H]⁺.

### (Z)-2-cyano-4,4,4-trifluoro-N-(2-(2-(2-methoxyethoxy)ethoxy)ethyl)-3-(6-(piperidin-1-yl)naphthalen-2-yl)but-2-enamide (3)

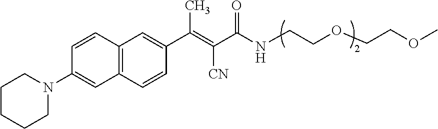

Compound (Z)-2-cyano-4,4,4-trifluoro-N-(2-(2-(2-methoxyethoxy)ethoxy)ethyl)-3-(6-(piperidin-1-yl)naphthalen-2-yl)but-2-enamide (3) was synthesized from 2,2,2-trifluoro-1-(6-(piperidin-1-yl)naphthalen-2-yl)ethan-1-one (9) (62 mg, 0.2 mmol) and α-cyanoamide (12) (55 mg, 0.24 mmol) according to the general procedure for Titanium-Catalyzed Knoevenagel Condensation. (Z)-2-cyano-4,4,4-trifluoro-N-(2-(2-(2-methoxyethoxy)ethoxy)ethyl)-3-(6-(piperidin-1-yl)naphthalen-2-yl)but-2-enamide (Compound 3) (36 mg, 35%) was obtained as an orange solid after purification by column chromatography on silica gel using hexane/EtOAc (30:70) as the eluent.

Rf=0.14 (hexane/EtOAc 50:50); ¹H NMR (400 MHz, Chloroform-d) δ 7.86 (s, 1H), 7.76-7.63 (m, 2H), 7.39-7.27 (m, 2H), 7.07 (d, J=17.7 Hz, 1H), 6.69 (t, J=5.6 Hz, 1H), 3.71-3.65 (m, 4H), 3.69-3.50 (m, 4H), 3.41-3.28 (m, 8H), 3.24 (q, J=5.2 Hz, 1H), 3.06 (dt, J=8.9, 4.5 Hz, 2H), 1.75 (dt, J=8.4, 4.4 Hz, 4H), 1.65 (q, J=5.8 Hz, 2H); ¹³C NMR (126 MHz, cdcl₃) δ 160.11, 159.66, 154.14, 135.86, 135.75, 129.60, 129.12, 128.79, 127.41, 127.16, 125.41, 125.12, 120.43, 114.65, 113.04, 71.92, 70.29, 69.91, 69.21, 68.99, 59.06, 40.30, 39.91, 29.79, 25.60, 24.33; ¹⁹F NMR (471 MHz, CHLOROFORM-D) δ −59.80, −61.39; HRMS 520.2418 calcd for [C₂₇H₃₃F₃N₃O₄]⁺, found 520.2421.

Upon synthesis and purification of compound (Z)-2-cyano-4,4,4-trifluoro-N-(2-(2-(2-methoxyethoxy)ethoxy)ethyl)-3-(6-(piperidin-1-yl)naphthalen-2-yl)but-2-enamide (3), a mixture of inseparable E and Z isomers were made, with an estimated 1.3:1 ratio of Z:E by NMR and assigned according to literature precedence³⁰. This issue with isomer mixture formation was observed previously with the synthesis of ARCAM, and does not appear to significantly affect the fluorescence and binding to amyloids²¹⁻²².

### Synthetic Procedure for Compounds (4 and 5)

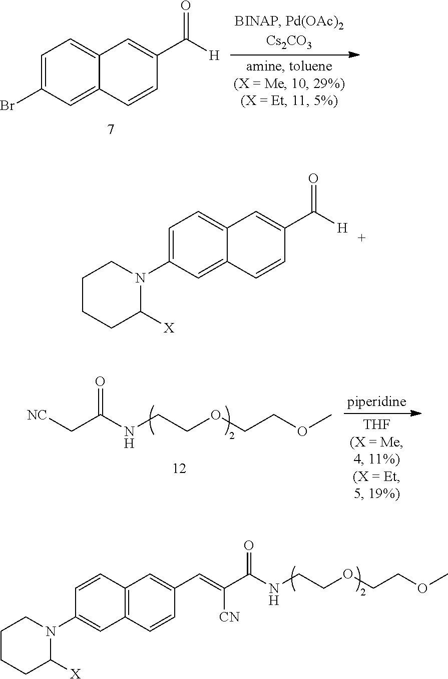

### 6-(2-methylpiperidin-1-yl)-2-naphthaldehyde (10)

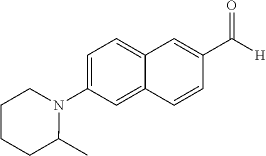 [SMILES: CC1CCCCN1c1ccc2cc(C=O)ccc2c1]

Aldehyde such as 6-(2-methylpiperidin-1-yl)-2-naphthaldehyde (10) was synthesized from compound 6-bromo-2-naphthaldehyde (7) (250 mg, 1.06 mmol) and racemic 2-methylpiperidine (0.15 mL, 1.28 mmol) according to the general procedure for Buchwald-Hartwig Coupling. 6-(2-methylpiperidin-1-yl)-2-naphthaldehyde (compound 10) (78 mg, 29%) was obtained as a yellow solid after purification by column chromatography on silica gel using hexane/EtOAc (90:10) as the eluent.

Rf=0.30 (hexane/EtOAc 90:10); ¹H NMR (500 MHz, Chloroform-d) δ 10.02 (s, 1H), 8.15 (d, J=1.7 Hz, 1H), 7.89-7.75 (m, 2H), 7.66 (d, J=8.5 Hz, 1H), 7.33 (dd, J=9.2, 2.5 Hz, 1H), 7.05 (d, J=2.5 Hz, 1H), 4.34 (p, J=5.7, 4.9 Hz, 1H), 3.59 (dt, J=12.8, 3.7 Hz, 1H), 3.08 (td, J=12.1, 3.1 Hz, 1H), 1.97-1.87 (m, 1H), 1.89-1.80 (m, 1H), 1.67 (m, 4H), 1.12 (d, J=6.8 Hz, 3H); ¹³C NMR (126 MHz, Chloroform-d) δ 192.13, 151.47, 138.77, 134.64, 131.25, 130.67, 127.27, 126.13, 123.50, 119.12, 109.09, 50.29, 42.54, 31.00, 25.89, 18.74, 13.61; ESI-MS: 254.32 [M+H]⁺.

### (E)-2-cyano-N-(2-(2-(2-methoxyethoxy)ethoxy)ethyl)-3-(6-(2-methylpiperidin-1-yl)naphthalen-2-yl)acrylamide (4)

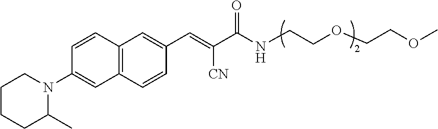

(E)-2-cyano-N-(2-(2-(2-methoxyethoxy)ethoxy)ethyl)-3-(6-(2-methylpiperidin-1-yl)naphthalen-2-yl)acrylamide (compound 4) was synthesized from 6-(2-methylpiperidin-1-yl)-2-naphthaldehyde (10) (78.3 mg, 0.31 mmol) and α-cyanoamide (12) (85.4 mg, 0.37 mmol) according to the general procedure for Knoevenagel Condensation. (E)-2-cyano-N-(2-(2-(2-methoxyethoxy)ethoxy)ethyl)-3-(6-(2-methylpiperidin-1-yl)naphthalen-2-yl)acrylamide (compound 4) (15 mg, 11%) was obtained as a yellow solid after purification by column chromatography on silica gel using hexane/EtOAc (20:80) as the eluent.

Rf=0.37 (100% EtOAc); ¹H NMR (500 MHz, Chloroform-d) δ 8.36 (s, 1H), 8.16 (d, J=1.9 Hz, 1H), 8.04 (dd, J=8.8, 1.9 Hz, 1H), 7.75 (d, J=9.2 Hz, 1H), 7.63 (d, J=8.8 Hz, 1H), 7.30 (dd, J=9.2, 2.4 Hz, 1H), 7.02 (d, J=2.4 Hz, 1H), 6.90 (t, J=4.8 Hz, 1H), 3.73-3.55 (m, 15H), 3.38 (s, 3H), 1.96-1.75 (m, 4H), 1.73-1.60 (m, 2H), 1.11 (d, J=6.8 Hz, 3H); ¹³C NMR (126 MHz, Chloroform-d) δ 161.48, 153.22, 151.30, 137.55, 134.04, 130.60, 127.25, 126.47, 125.99, 125.85, 119.10, 118.06, 108.89, 100.33, 72.04, 70.73, 70.61, 69.62, 59.18, 50.24, 42.50, 40.34, 30.96, 29.83, 25.86, 18.71, 13.66. HRMS 466.2706 calcd for [C₂₇ H₃₅ N₃O₄]⁺, found 466.2697.

### 6-(2-ethylpiperidin-1-yl)-2-naphthaldehyde (11)

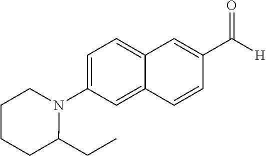 [SMILES: CCC1CCCCN1c1ccc2cc(C=O)ccc2c1]

Aldehyde such as 6-(2-ethylpiperidin-1-yl)-2-naphthaldehyde (11) was synthesized from 6-bromo-2-naphthaldehyde (compound 7) (250 mg, 1.06 mmol) and racemic 2-ethylpiperidine (0.17 mL, 1.28 mmol) according to the general procedure for Buchwald-Hartwig Coupling. 6-(2-ethylpiperidin-1-yl)-2-naphthaldehyde (compound 11) (14 mg, 5%) was obtained as a yellow solid after purification by column chromatography on silica gel using hexane/EtOAc (90:10) as the eluent.

Rf=0.63 (hexane/EtOAc 80:20); ¹H NMR (500 MHz, Chloroform-d) δ 10.01 (s, 1H), 8.13 (s, 1H), 7.80 (t, J=9.0 Hz, 2H), 7.63 (d, J=8.7 Hz, 1H), 7.30 (d, J=9.3 Hz, 1H), 7.01 (s, 1H), 4.01 (s, 1H), 3.66 (d, J=13.0 Hz, 1H), 3.11 (t, J=12.1 Hz, 1H), 1.78 (s, 2H), 1.71-1.61 (m, 6H), 0.89 (t, J=7.5 Hz, 3H); ¹³C NMR (126 MHz, Chloroform-d) δ 192.10, 154.07, 151.65, 138.92, 134.67, 132.26, 131.02, 130.74, 128.70, 127.09, 125.78, 123.55, 118.84, 108.32, 56.79, 42.51, 29.84, 27.24, 25.43, 21.01, 18.92, 11.64; ESI-MS: 268.29 [M+Na]⁺.

### (E)-2-cyano-3-(6-(2-ethylpiperidin-1-yl)naphthalen-2-yl)-N-(2-(2-(2-methoxyethoxy)ethoxy)ethyl)acrylamide (5)

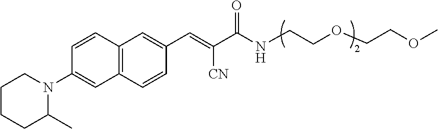

(E)-2-cyano-3-(6-(2-ethylpiperidin-1-yl)naphthalen-2-yl)-N-(2-(2-(2-methoxyethoxy)ethoxy)ethyl)acrylamide (5) was synthesized from Compound 6-(2-ethylpiperidin-1-yl)-2-naphthaldehyde 11 (14 mg, 0.05 mmol) and α-cyanoamide (12) (15 mg, 0.06 mmol) according to the general procedure for Knoevenagel Condensation. Compound (E)-2-cyano-3-(6-(2-ethylpiperidin-1-yl)naphthalen-2-yl)-N-(2-(2-(2-methoxyethoxy)ethoxy)ethyl)acrylamide (5) (4.5 mg, 19%) was obtained as an orange solid after purification by column chromatography on silica gel using hexane/EtOAc (20:80) as the eluent.

Rf=0.59 (100% EtOAc); ¹H NMR (400 MHz, Chloroform-d) δ 8.36 (s, 1H), 8.15 (s, 1H), 8.03 (d, J=8.8 Hz, 1H), 7.64 (m, 2H), 7.28 (s, 1H), 6.97 (d, J=14.5 Hz, 1H), 6.87 (s, 1H), 3.73-3.31 (m, 18H), 1.78 (s, 2H), 0.89 (t, J=6.9 Hz, 3H); ¹³C NMR (126 MHz, cdcl₃) δ 161.51, 154.16, 153.25, 151.52, 137.72, 134.15, 130.69, 127.09, 125.90, 118.86, 118.16, 108.16, 99.98, 72.07, 70.74, 69.62, 59.22, 56.75, 42.48, 40.33, 32.07, 29.85, 27.26, 25.44, 21.09, 18.91, 14.30, 11.64; HRMS 480.2857 calcd for [C₂₈ H₃₈ N₃ O₄]⁺, found 480.2853.

### Example 3

### Results and Discussion

Within the ARCAM scaffold, there are two potential rotatable bonds between the D and A that may affect fluorescence intensity, one between the piperidine and 6th position of the naphthalene group (rotatable bond a, FIG. 1B) and one between the vinyl and the 1 position of the naphthalene group (rotatable bond b). In order to influence the rotational barrier and planarity of the molecule at rotatable bond a, ARCAM analogs 4 and 5 (FIG. 1C) were designed and synthesized, which contained either a methyl or ethyl group on the 2-piperidinyl position of the ARCAM scaffold. To affect the rotation and planarity of rotatable bond b, ARCAM analogs 2 and 3 (FIG. 1C) were designed and synthesized compounds, which comprised either a methyl or trifluoromethyl group on the vinylic position of the ARCAM scaffold.

The synthesis of ARCAM (1) was reported previously.²¹ The syntheses of ARCAM analogs (2-5) are shown in Scheme 1. Briefly, commercially available methyl 6-bromo-2-naphthoate (6) was converted to the corresponding bromoaldehyde (7) by reduction of the ester using Diisobutylaluminium Hydride (DIBAL-H), followed by oxidation of the primary alcohol to the aldehyde using (Pyridinium Chlorochromate) PCC. Intermediate bromoaldehyde such as 6-bromo-2-naphthaldehyde (7) was then further reacted in three ways: 1) for the synthesis of piperidinylketone such as 1-(6-(piperidin-1-yl)naphthalen-2-yl)ethan-1-one (8) through nucleophilic attack with methyl Grignard, oxidation to the ketone, and Buchwald-Hartwig coupling with piperidine, 2) for the synthesis of trifluoromethylketone such as 2,2,2-trifluoro-1-(6-(piperidin-1-yl)naphthalen-2-yl)ethan-1-one (9) by first Buchwald-Hartwig coupling with piperidine, followed by nucleophilic attack with (trifluoromethyl)trimethylsilane and oxidation, or 3) for the synthesis of piperidinylaldehydes such as 6-(2-methylpiperidin-1-yl)-2-naphthaldehyde (10) and 6-(2-ethylpiperidin-1-yl)-2-naphthaldehyde (11) through Buchwald-Hartwig coupling with 2-methyl or 2-ethyl piperidine, respectively. Intermediates piperidinylketone-piperidinylaldehydes (8-11) were then subjected to Knoevenagel condensation with previously reported²¹ α-cyanoamide (12) to afford fluorescent ARCAM analogs (2-5). Due to the limitations on commercial availability of enantiomerically pure 2-substituted piperidines, ARCAM analogs (4 and 5) were generated as racemic mixtures in this initial study.

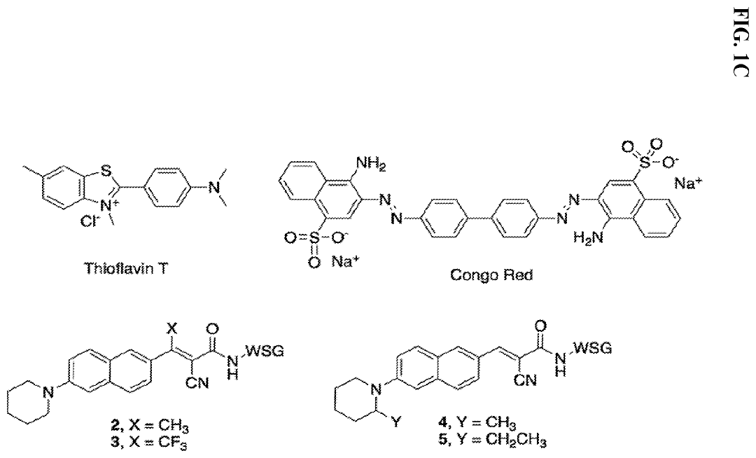

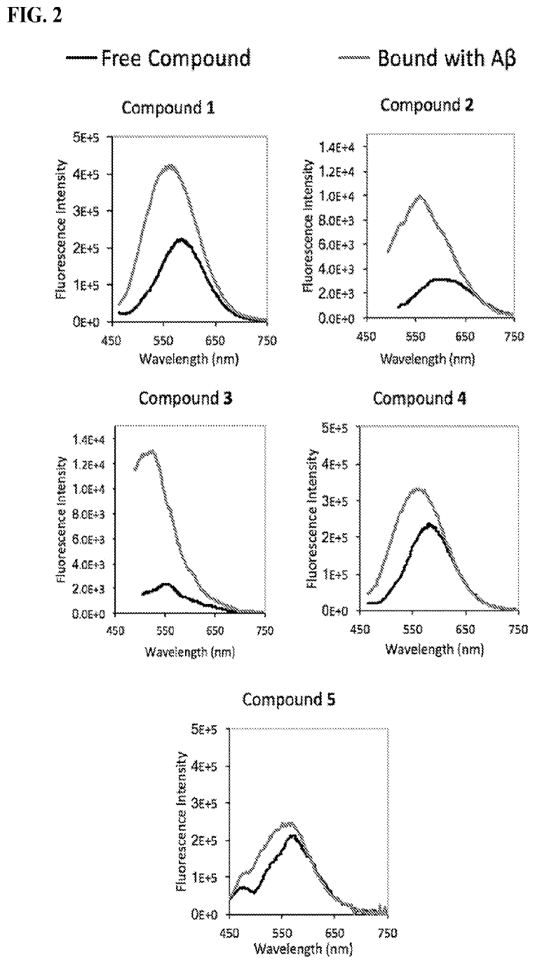

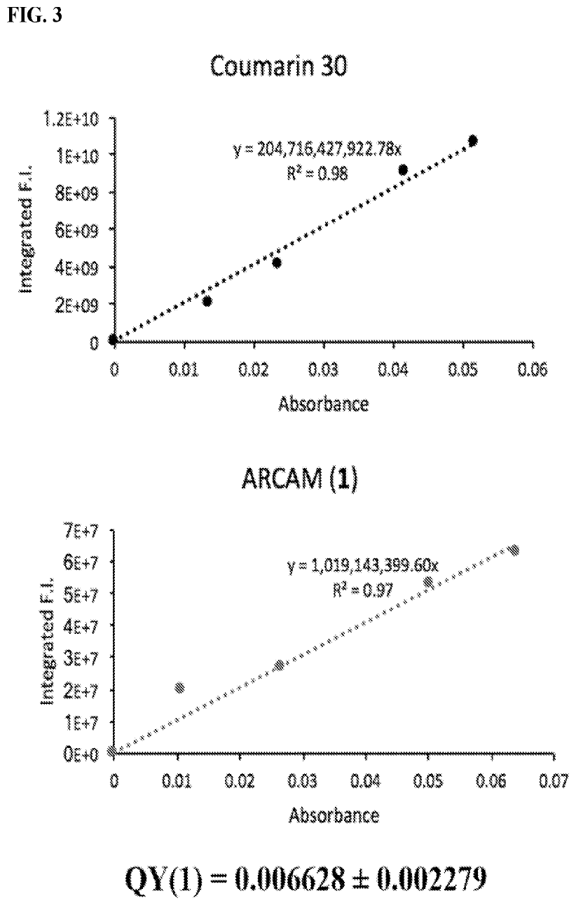

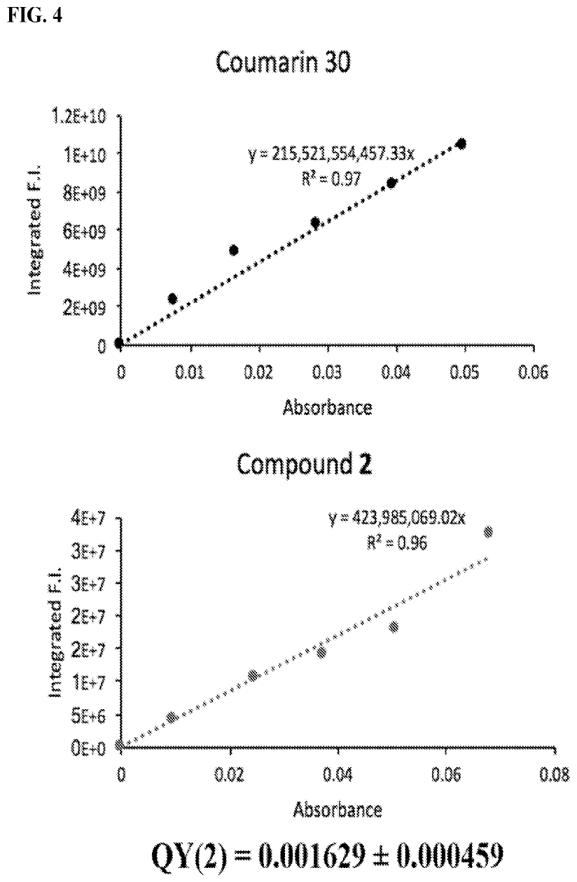

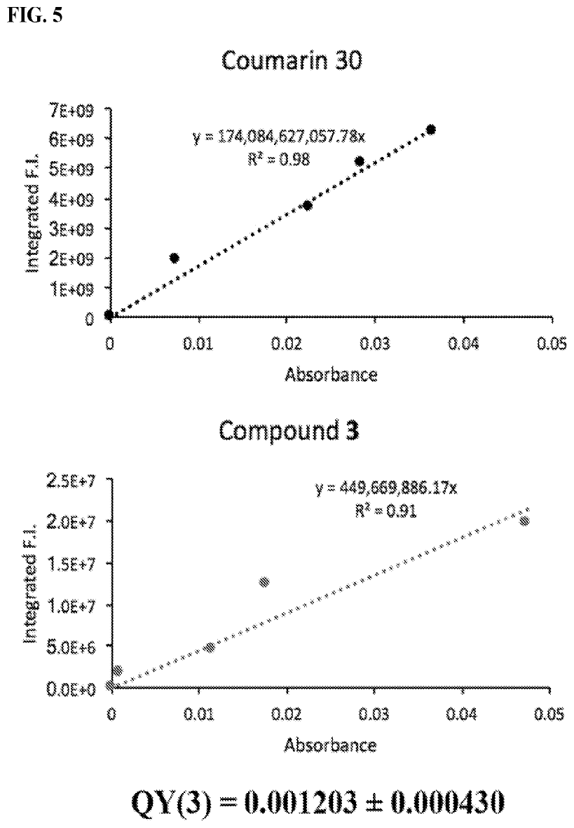

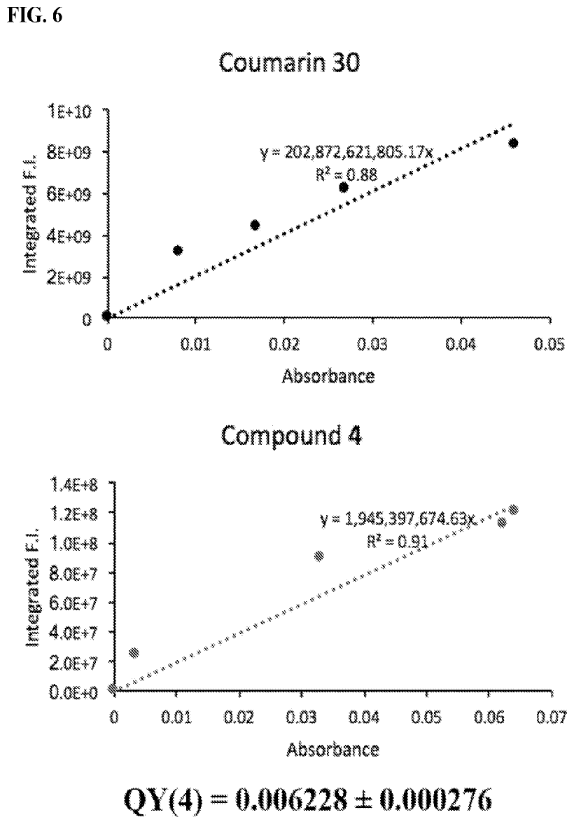

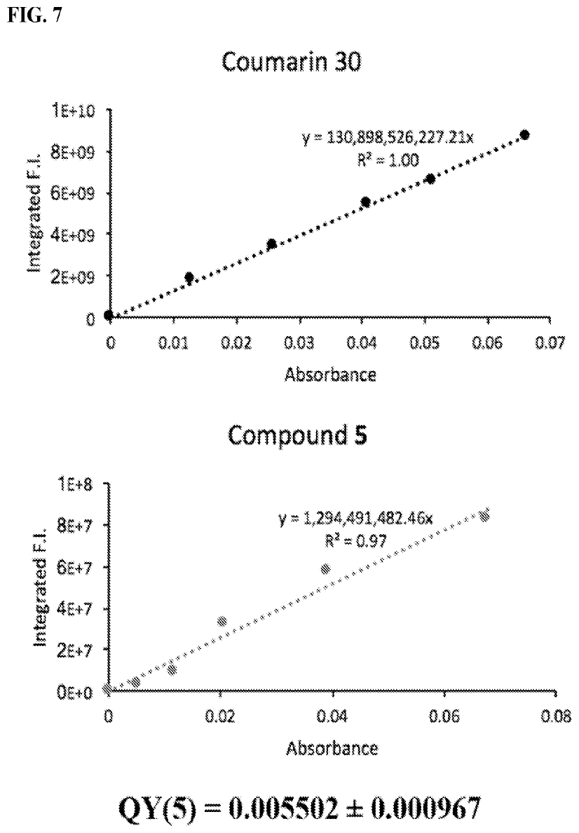

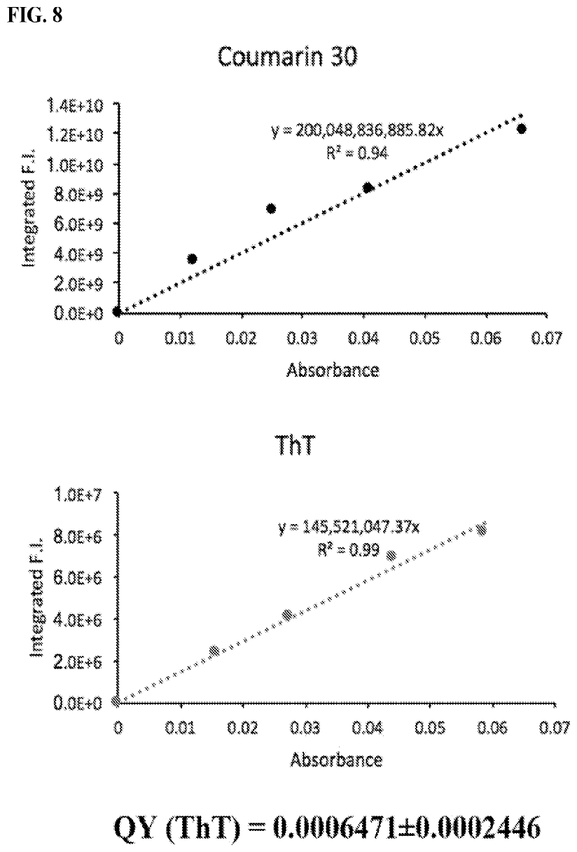

With ARCAM analogs, (1-5), aliphatic substituents near the rotatable single bonds a and b on the ARCAM scaffold (FIG. 1B) had an effect on the QY of the free probes in solution compared to the parent ARCAM compound (FIGS. 3-7). The absorption and emission spectra of analogs of ARCAM, (1-5) in aqueous solution was measured by using a similar protocol as previously described for estimation of the QY of ThT.²⁴ Table 1 summarizes the estimates for QY for analogs ARCAM, (1-5). These solution studies revealed that the QY of ARCAM (1) is an order of magnitude higher than the QY estimated for ThT (0.0006) (FIG. 8). Interestingly, in all cases where aliphatic substituents on the ARCAM scaffold were introduced, the QY decreased compared to the parent ARCAM (1), with the substituents on the vinylic position causing a larger decrease in QY than substituents on the piperidine ring (Table 1). This trend of decreased QY for substituted ARCAM analogs was reflected in the relative intensity of the fluorescence emission spectra of the free probes (black lines, FIG. 2) in aqueous solution, as compounds 2 and 3 decreased in fluorescence intensity by two orders of magnitude compared to ARCAM (1), while the fluorescence intensity of free compounds 4 and 5 in solution were lower but similar in magnitude to ARCAM (1).

TABLE 1
Spectroscopic and amyloid-binding characteristics of compounds 1-5.

| | | Ex | | | | | |
|---|---|---|---|---|---|---|---|
| | | (λₘₐₓ) | Em | | | | |
| | Ex (λₘₐₓ) | (with | (λₘₐₓ) | Em (λₘₐₓ) | Fold | | |
| Cmpd | (Free) | Aβ) | (Free) | (with Aβ) | Increase | K_{D} with | |
| no. | (nm) | (nm) | (nm) | (nm) | with Aβ | Aβ (μM) | QY |
| 1 | 409 ± 11 | 421 ± 4 | 590 ± 4 | 560 ± 10 | 2.1 ± 0.7 | 6.3 ± 0.7 | 0.0066 ± 0.0023 |
| 2 | 372 ± 14 | 364 ± 6 | 601 ± 2 | 547 ± 4 | 3.5 ± 0.9 | 5.0 ± 1.7 | 0.0016 ± 0.0005 |
| 3 | 419 ± 4 | 410 ± 5 | 547 ± 5 | 514 ± 6 | 5.4 ± 0.9 | 3.7 ± 1.1 | 0.0012 ± 0.0004 |
| 4 | 431 ± 4 | 439 ± 3 | 583 ± 1 | 559 ± 8 | 1.9 ± 0.5 | 4.4 ± 2.3 | 0.0062 ± 0.0003 |
| 5 | 445 ± 1 | 443 ± 5 | 575 ± 6 | 567 ± 5 | 1.2 ± 0.1 | 3.2 ± 0.6 | 0.0055 ± 0.0010 |

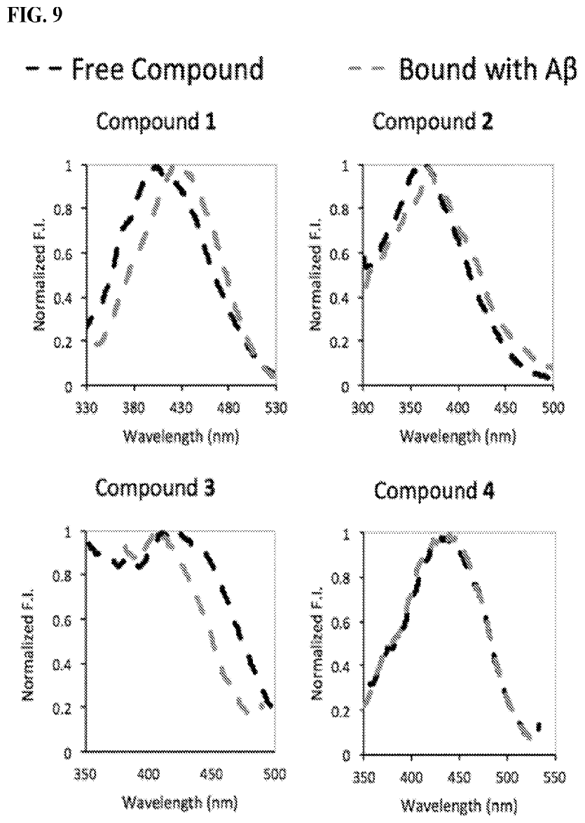

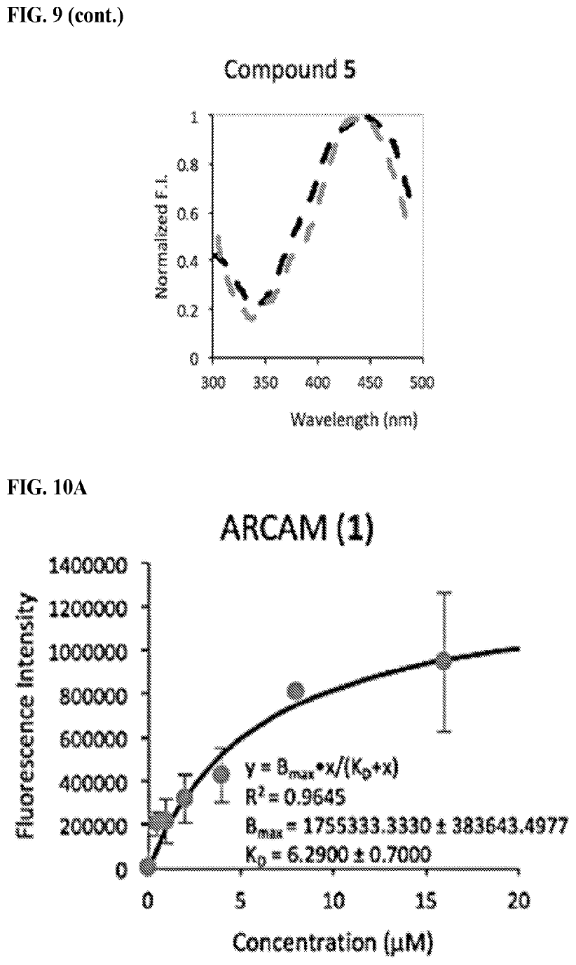

In order to probe whether the aliphatic substituents could introduce any electronic effects on the spectroscopic properties of ARCAM analogs, the excitation (FIG. 9) and emission profiles (black lines, FIG. 2) of analogs of ARCAM 1-5 were compared. Introduction of the methyl or ethyl group on the piperidine ring (as in probes 4 and 5, respectively) led to a bathochromic or red-shift in the excitation maximum (Table 1) relative to ARCAM (1). These observations suggest that introduction of α-donating groups near the electron donating nitrogen within the piperidine group lowers the energy required for photoexcitation. Introduction of substituents on the vinylic position of the ARCAM scaffold also exhibited an effect on excitation maxima, where a σ-donating methyl group on the vinylic position (as in probe 2) led to a hypsochromic or blue-shift in excitation maximum and a σ-withdrawing trifluoromethyl group on the vinylic position (as in probe 3) led to a bathochromic shift in excitation maximum compared to parent ARCAM (1); the effects of substituents at the vinylic position in probes 2 and 3 are consistent with the expectation that σ-donating groups near the electron acceptor of the fluorophore will increase the energy required to generate the photoexcited state and σ-withdrawing groups will have the opposite effect. For the effects of substituents on emission profiles, introduction of the methyl group in 2 resulted in a bathochromic shift in the maximum emission wavelength (λₘₐₓ) of the free probe in solution compared to the parent ARCAM (1) (Table 1), whereas introduction of the trifluoromethyl group in 3 resulted in a hypsochromic shift in emission λₘₐₓ compared to 1. These observations suggest that electron donating groups (e.g., the methyl group in 2) near the electron acceptor region (e.g., the nitrile group in 1-5) of the molecular rotor can help stabilize the dipolar photoexcited LE state leading to a lower energy (or higher wavelength) of photon emission, whereas electron withdrawing groups (e.g., the trifluoromethyl group in 3) at the same position leads to destabilization of the LE state and a higher energy (or lower wavelength) of photon emission upon relaxation to the ground state. Electron donating substituents on the 2-piperidinyl position of the ARCAM scaffold (e.g., the methyl or ethyl groups in 4 and 5), on the other hand, apparently increased the energy of fluorescence emission of the free probe compared to the parent ARCAM probe, albeit the effect was relatively small. The excitation and emission λₘₐₓ of 1-5 spanned a range of 73 nm and 54 nm, respectively, demonstrating that additions of small aliphatic groups on the ARCAM scaffold can lead to very large changes in spectral characteristics of these fluorophores and can help fine tune the spectroscopic properties of these probes for specialized applications.^{14,16,17,25}

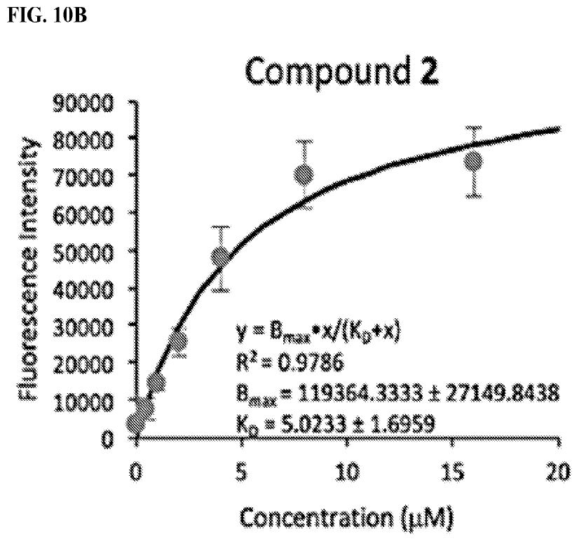

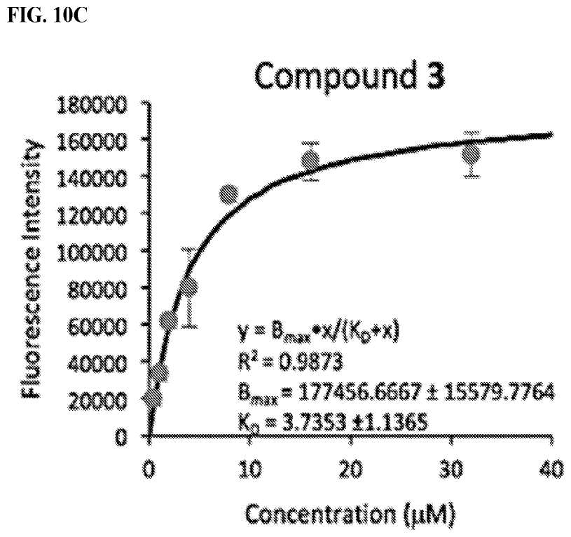

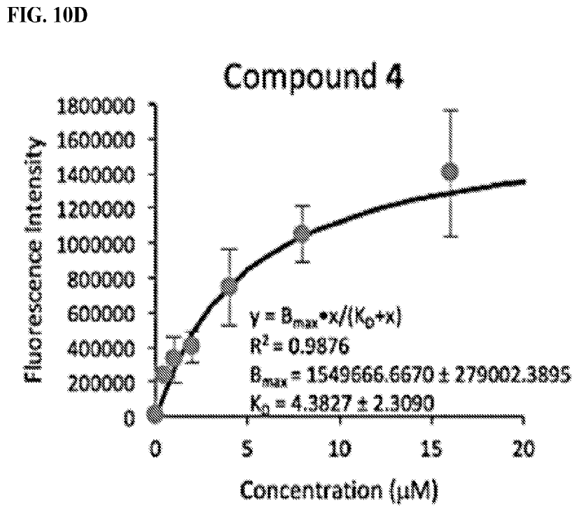

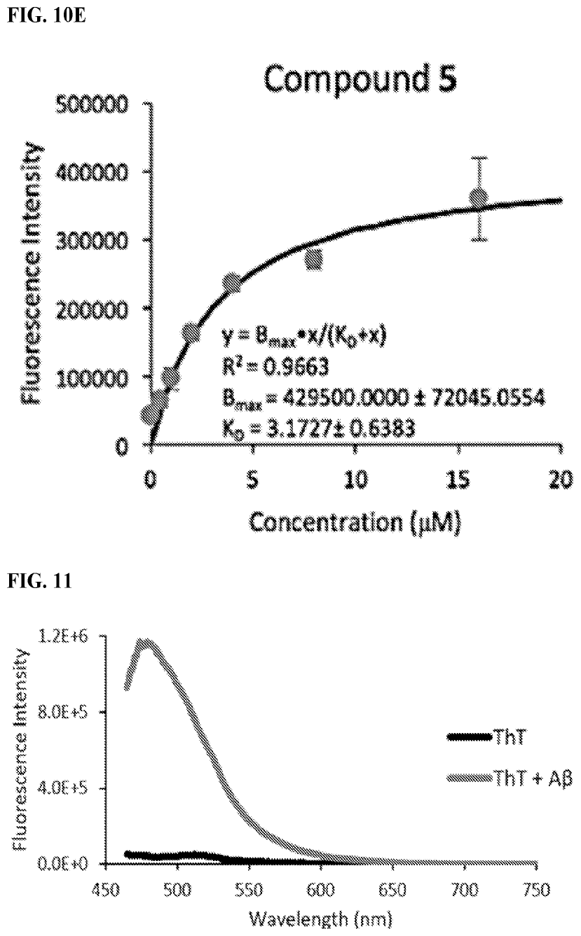

In order to examine the binding and fluorescence properties of ARCAM and its analogs in the presence of Aβ aggregates, a solution of aggregated Aβ (1-42) peptides was prepared by using a previously reported protocol (see the Methods section for details of this preparation and FIGS. 3-12 for characterization)²⁶. Binding measurements²¹ revealed that all of the analogs of ARCAM, 1-5 bound with similar low micromolar affinities to aggregated Aβ (Table 1 and FIG. 10), demonstrating that small aliphatic substituents on the ARCAM scaffold do not significantly affect binding to amyloids. However, substantial differences were found in the fluorescence enhancement properties between these probes in amyloid-containing versus amyloid-free solutions. For the parent compound ARCAM (1), a 2.1-fold increase was observed in fluorescence intensity in the presence of aggregated Aβ compared to background fluorescence of the probe in the absence of Aβ (FIG. 2 and Table 1). For analogs of ARCAM, 2 and 3, containing substituents on the vinylic group, a 3.5- and 5.4-fold increase was found in fluorescence intensity in the presence of aggregated Aβ compared to background fluorescence, respectively. ARCAM analogs such as new fluorescent ARCAM 4 and 5 with substituents on the piperidine ring, on the other hand, exhibited only a 1.9 and 1.2-fold increase in fluorescence intensity in the presence of aggregated Aβ compared to background, respectively. While the changes in the maximal excitation wavelength of all probes varied upon binding to aggregated Aβ (Table 1 and FIG. 9), all of the probes displayed a hypsochromic shift in emission λₘₐₓ in the presence of aggregated Aβ compared to free probes in solution. These observations are consistent with previous studies on ARCAM (1) and suggest that spectroscopic measurements for all of the probes in the presence of aggregated Aβ were dominated by their fluorescence properties in the bound state.^{14,15,17-21} These results also demonstrate that the location and the identity of the substituent on the ARCAM scaffold is important for exhibiting the overall effect on fluorescence enhancement of these probes in the presence of aggregated Aβ. While aliphatic substituents at the vinylic position on ARCAM significantly lowered the background fluorescence of these molecules, these substituents apparently had a larger effect on increasing the relative fluorescence intensity when bound to an amyloid. Conversely, aliphatic substituents on the 2-position of the piperidine ring of ARCAM appear to only lower the fluorescence enhancement properties of the probes when bound to amyloids, without any substantial effect on the fluorescence of the free probes in solution.

In conclusion, aliphatic substituents near the rotatable bonds of ARCAM can affect the fluorescence properties of probes both free in solution and when bound to aggregated Aβ. When substituents were introduced on the vinylic position of the ARCAM scaffold, the overall brightness of the probes were lowered, but the enhancement upon binding to aggregated Aβ compared to background was increased. Electronic effects of aliphatic substituents on this position of ARCAM can affect the emission wavelength of the free probe, with a red-shift or blue-shift depending on whether the group is electron donating or electron withdrawing, respectively. Such changes in emission wavelength are typically achieved by varying the extent of conjugation in the π-framework of the D-π-A motif of molecular rotors,¹⁴ rather than structural changes as simple as introduction of an aliphatic group outside of the π system. When introducing aliphatic substituents on the 2-piperidinyl position of the ARCAM saffold, the overall brightness of the free probe in solution was essentially unchanged, but the relative fluorescence intensity of these probes upon binding to aggregated Aβ was decreased compared to the parent ARCAM compound. Probes 1-5 all had similar binding affinities to aggregated Aβ, supporting that the changes in spectroscopic properties observed in the presence of aggregated Aβ were not due to differences in the amyloid-binding capabilities of these compounds. This study shows that considering the rotational freedom in the design of molecular rotors can lead to fluorescent amyloid-targeting probes with increased contrast between bound and unbound states, which may serve as new design principles for tuning both their spectroscopic as well as fluorescence enhancement properties for development as chemical tools to aid in the diagnosis of amyloid-associated diseases.

### Example 4

### Methods

Synthesis and Characterization of ARCAM Analogs. The synthesis of ARCAM (1) was reported previously.²¹ The Supporting Information summarizes the synthesis and characterization for ARCAM analogs (2-5).

Quantum Yield Measurements. To determine the quantum yield (QY) of compounds ARCAM, 1-5, a procedure that was similar to a previously reported protocol for estimating the QY for Thioflavin T²⁴ was used. Briefly, the absorbance spectrum of each probe (in 5% Dimethyl Sulfoxide (DMSO)/water (H₂O)), in addition to the absorbance spectrum for the reference standard (Coumarin 30, QY=0.67)²⁷ was measured. The wavelength at which the two normalized absorbance curves intersected was used as the excitation wavelength. A serial dilution of each probe and the standard was made with absorbance values at the excitation wavelength in the range of 0 to 0.1 absorbance units. At each concentration, the emission spectra for each probe and the standard were measured. Technical triplicates of these experiments were performed for all probes, and the averages were recorded. From these emission spectra, the area under the curve was calculated and plotted against the absorbance at each concentration (FIGS. 3-7). The data were fitted to Equation 1 to obtain estimates for QY:

$${QY}_{p} = {QY}_{r} ( \frac{{A}_{r}}{{A}_{p}} ) ( \frac{{E}_{p}}{{E}_{r}} ) {( \frac{{η}_{p}}{{η}_{r}} )}^{2} \qquad ( 1 )$$

where QY represents the quantum yield, A represents the absorbance, E represents the integrated fluorescence emission, η represents the refractive index of the solvent^{28,29}, and the ‘p’ and ‘r’ subscripts signify the probe or the reference compound, respectively.

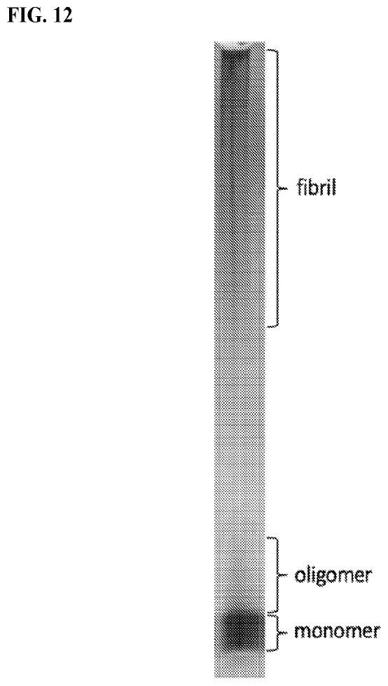

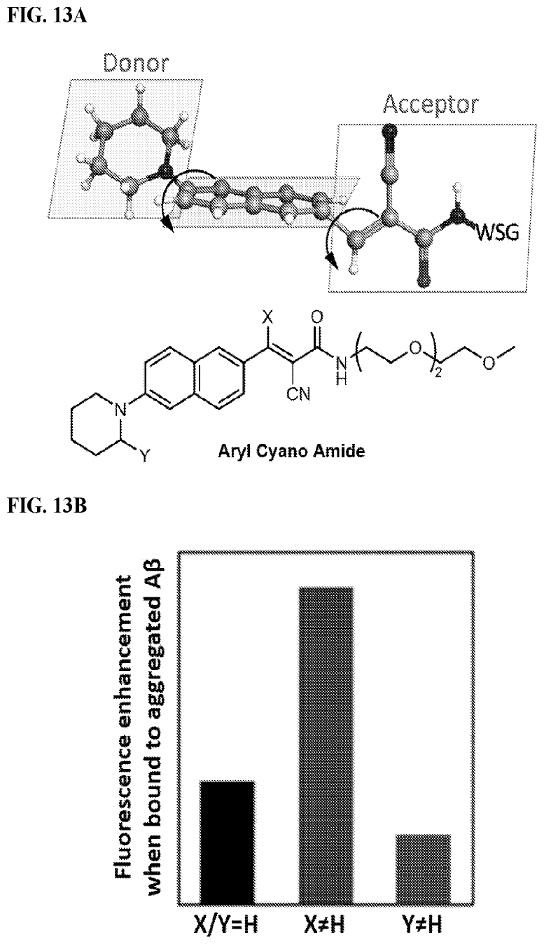

Preparation of aggregated Aβ (1-42) peptides. Synthetic Aβ (1-42) peptide was purchased from Biopeptide, Co LLC (San Diego, California). Aggregated Aβ (1-42) was prepared as previously described.²⁶ Briefly, Aβ (1-42) was dissolved in 100% 1,1,1,3,3,3,-hexafluoro-2-propanol (HFIP) to a concentration of 1 mM and put on a shaker at room temperature (RT) for 24 hours. The solution was then diluted in cold nanopure water (2:1 H₂O: HFIP). Aliquoted fractions were lyophilized for 3 days before dissolving in nanopure water to a concentration of 100 μM. Aggregated Aβ solutions were incubated and shaken at 37° C. for 3 days before use. The formation of soluble aggregates (67% fibrils as a mixture with 13% oligomers and 20% monomer) was confirmed using a standard thioflavin T (ThT) assay and by Sodium Dodecyl Sulphate-Polyacrylamide Gel Electrophoresis (SDS-PAGE) gel analysis (FIG. 11 and FIG. 12).

Estimation of Binding Constant (K_{D}) to aggregated Aβ (1-42) peptides. Aggregated Aβ(1-42) at a final concentration of 5 μM (based on the molecular weight of monomer) was mixed with increasing concentration of each probe in 5% DMSO in nanopure water. KD's were determined as previously described.²¹

Estimation of fluorescence enhancement of probes when bound versus unbound to aggregated Aβ (1-42) peptides. Aggregated Aβ (1-42) at a final concentration of 5 μM (based on the molecular weight of monomer) was mixed with each fluorescent probe at a final concentration of 4 μM in 5% DMSO in nanopure water. The solution (80 μL) was transferred to a black opaque 96 well plate and the fluorescence was read using a microplate reader. For all probes, the relative fluorescence enhancement of the bound versus unbound state was estimated by comparing the intensity at λₘₐₓ of the amyloid-bound state.

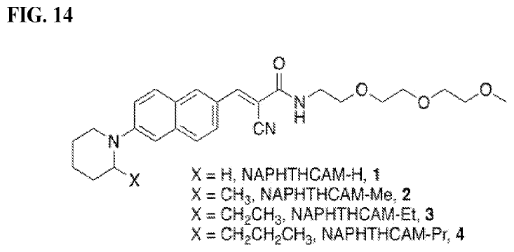

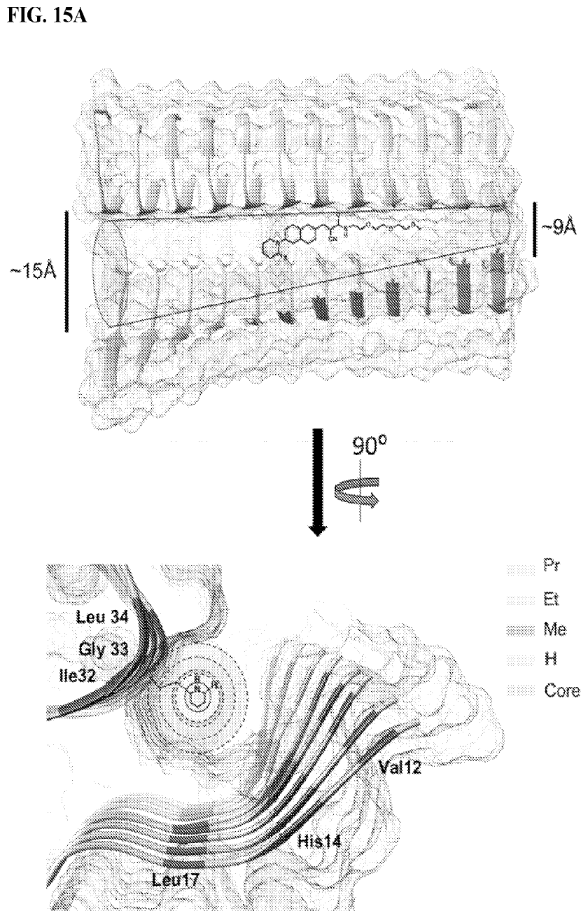

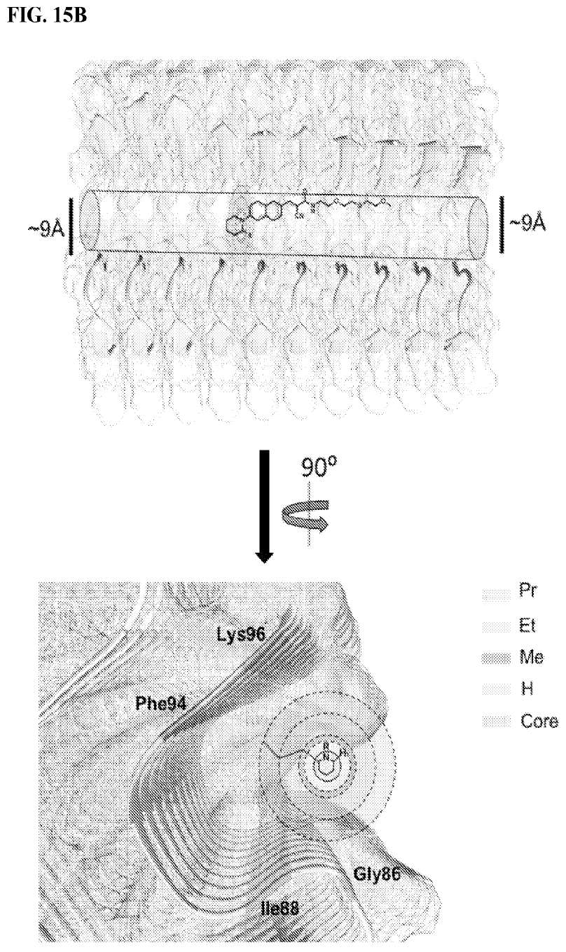

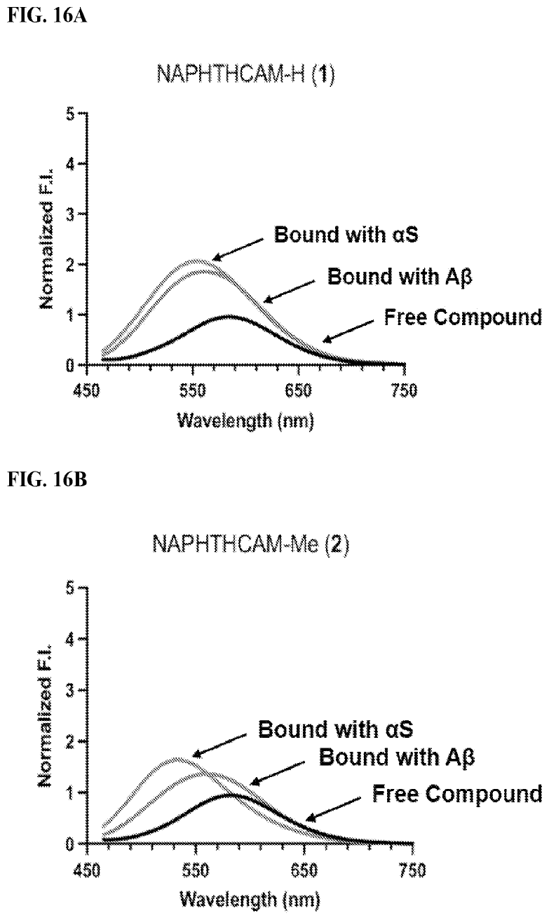

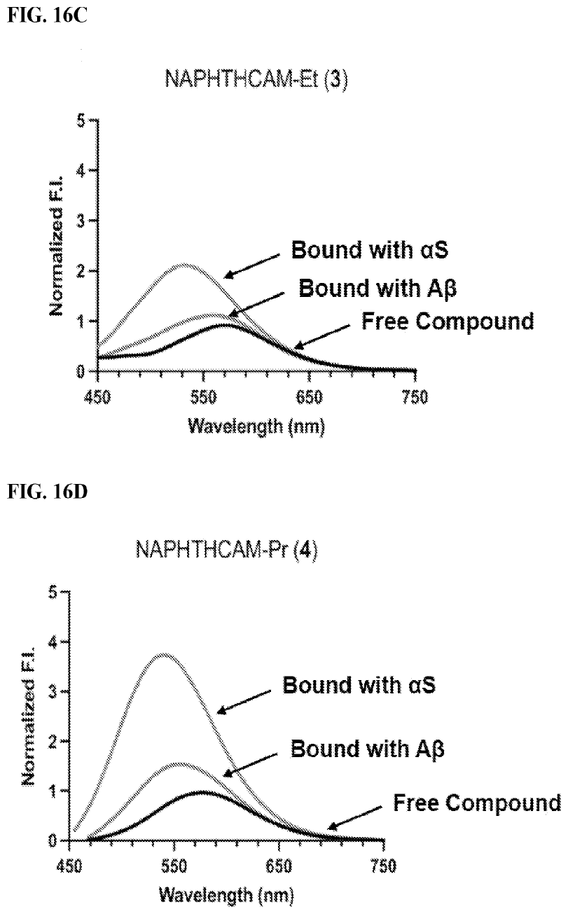

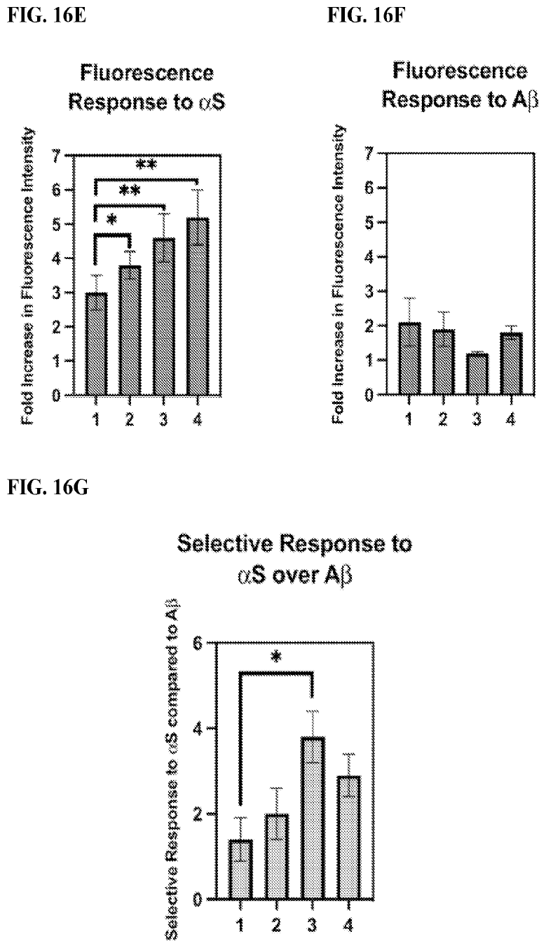

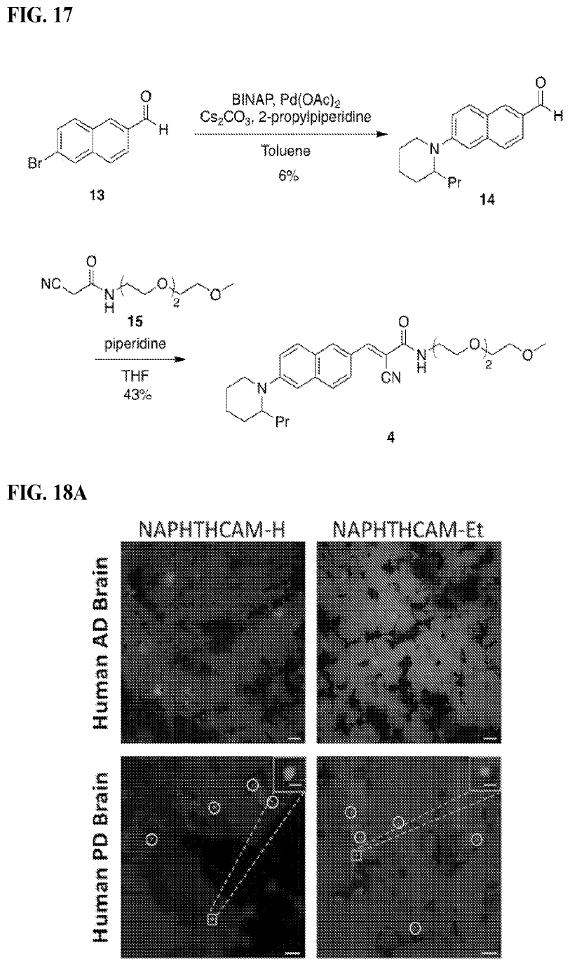

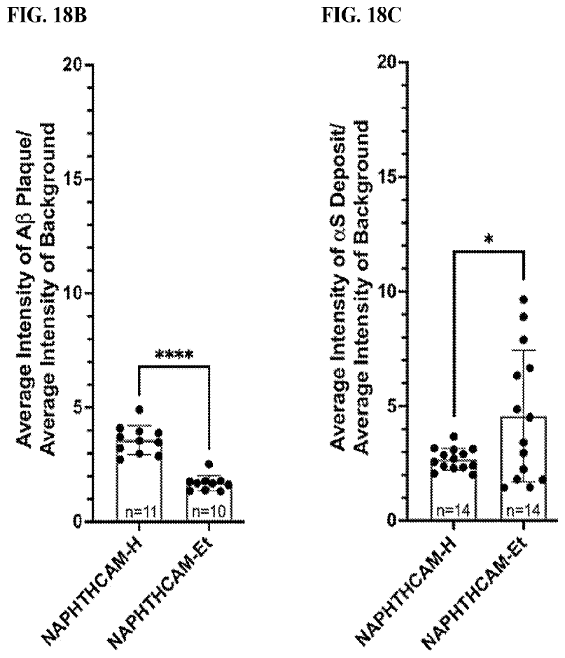

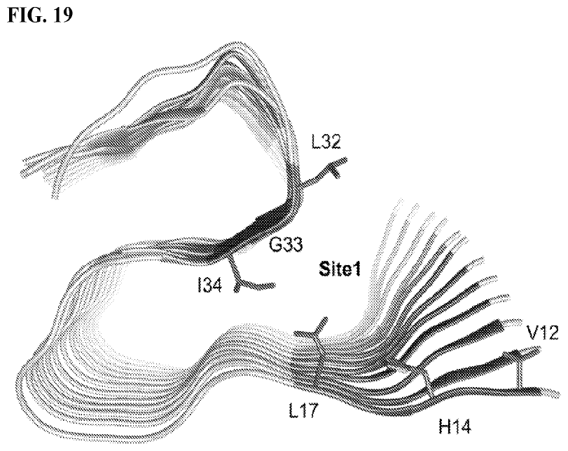

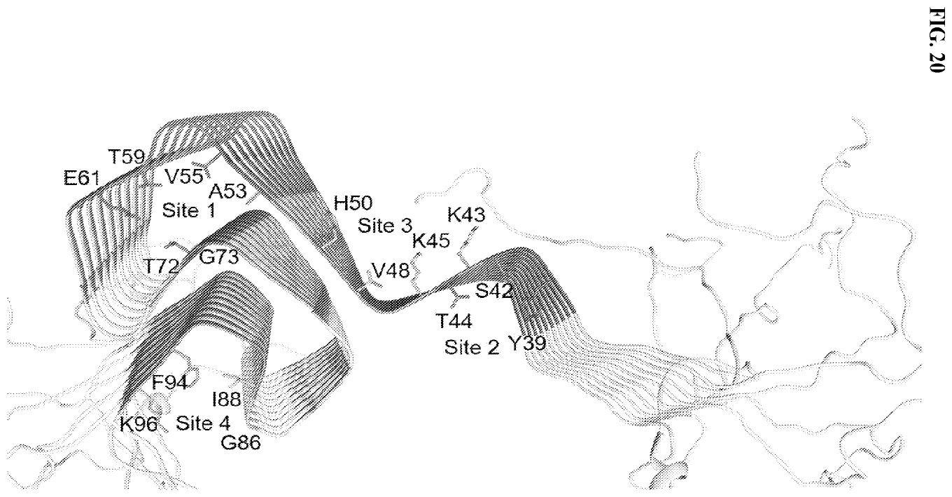

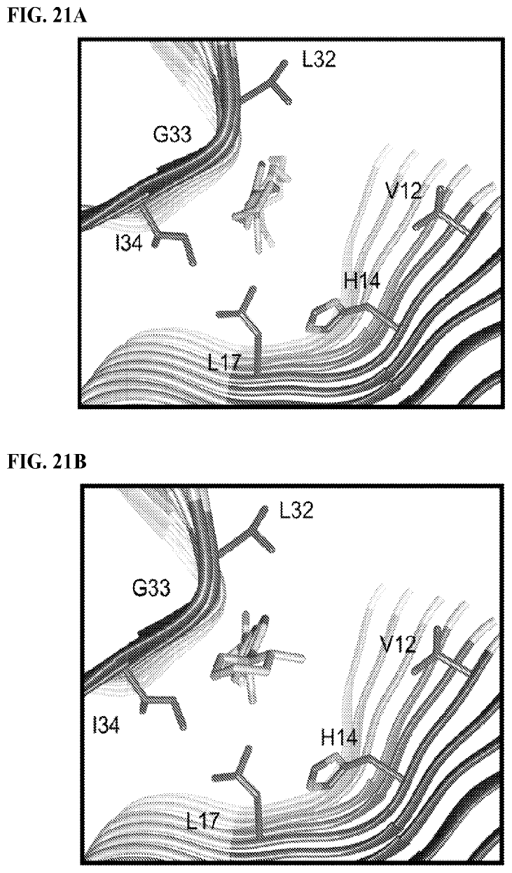

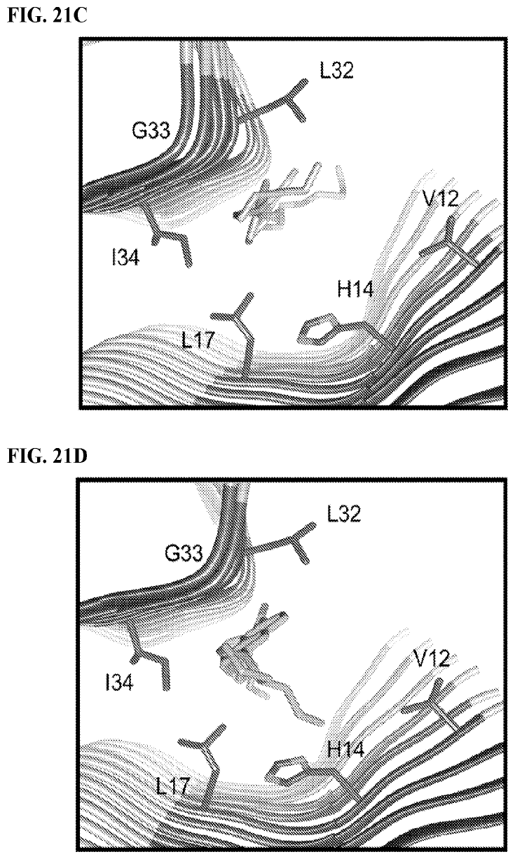

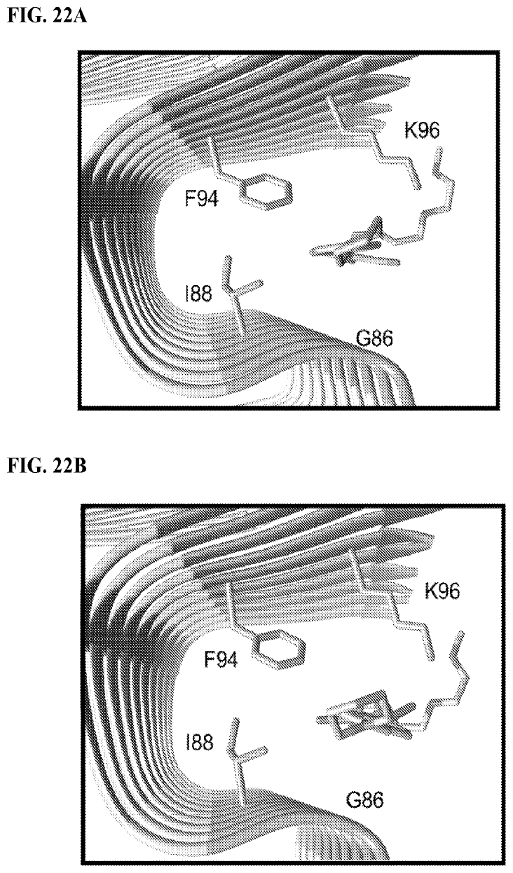

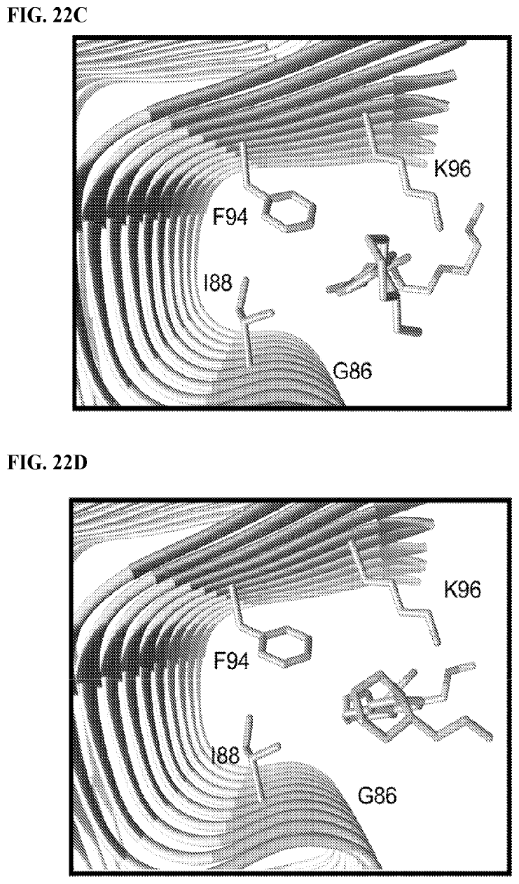

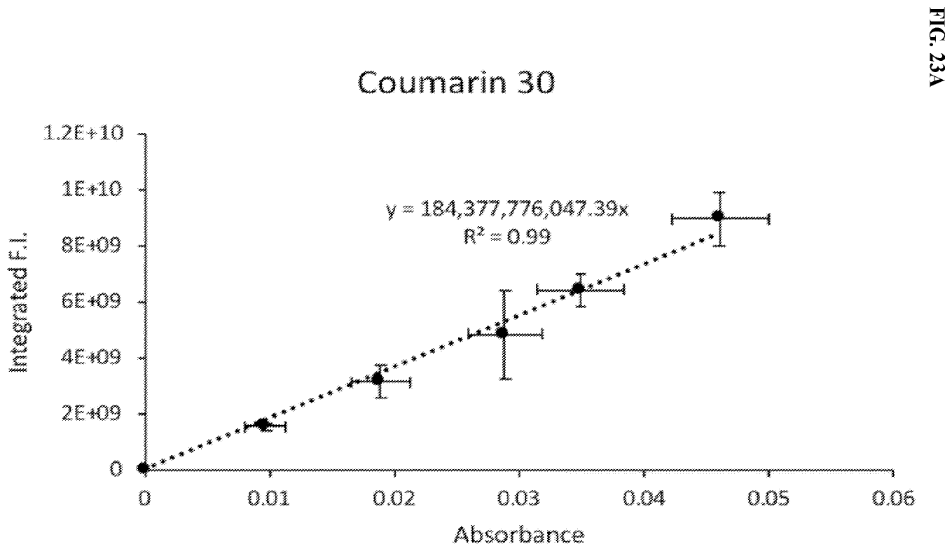

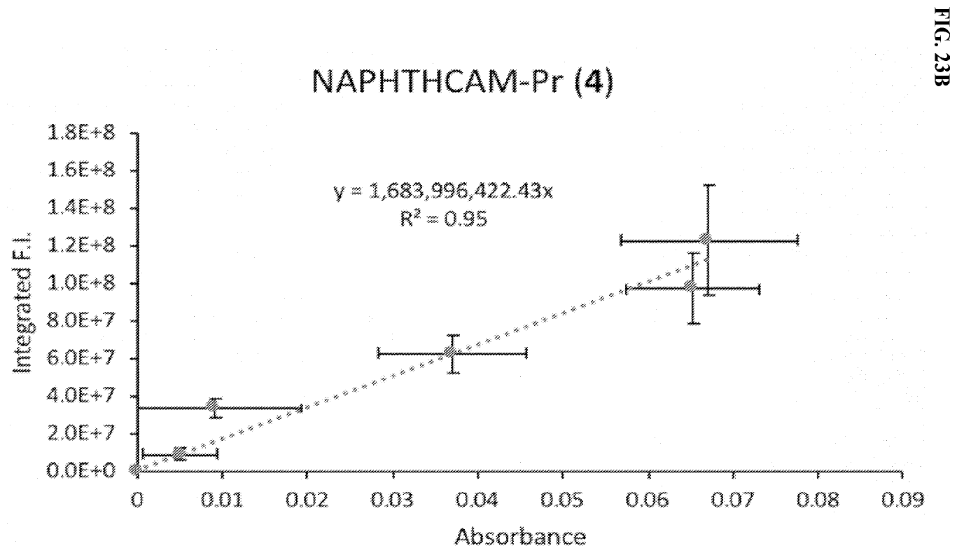

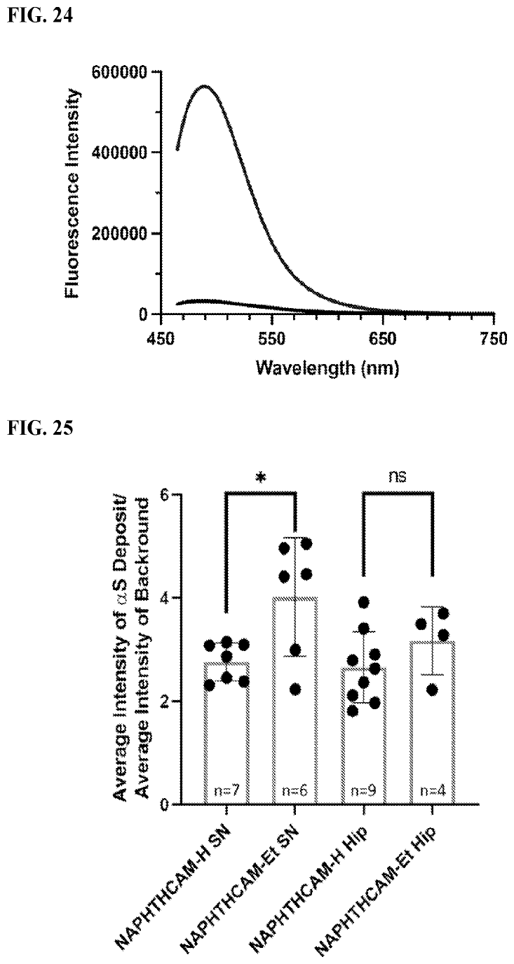

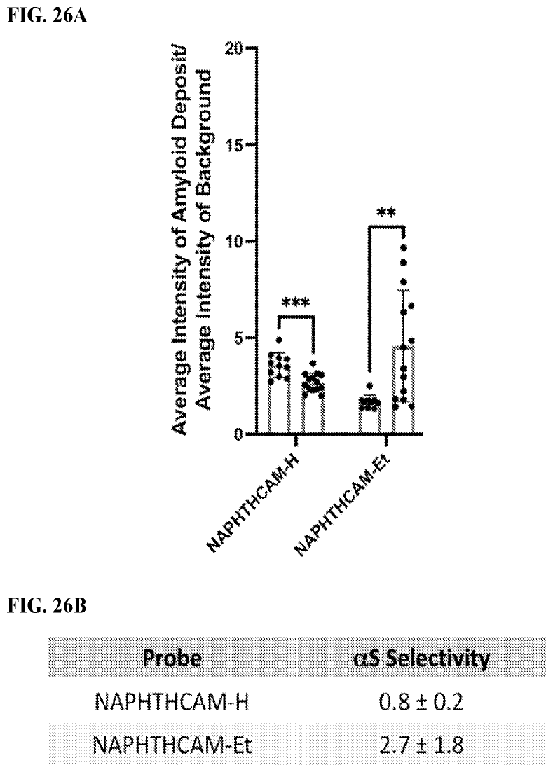

### Example 5: Experimental Procedures and Characterization Data for Compounds in FIG. 14

Probes 1, 2, and 3 were synthesized as previously reported.³¹ All reagents were purchased from commercial sources and used without further purification except where noted. Air- and moisture-sensitive liquids and solutions were transferred via syringe under argon atmosphere. Organic solutions were concentrated by rotary evaporation below 45° C. at approximately 20 mmHg. All non-aqueous reactions were carried out under anhydrous conditions. Reactions were monitored by thin-layer chromatography (TLC) carried out on 0.25 mm Dynamic Adsorbents, Inc. silica gel plates (60F-254) and visualized under UV light and/or developed by dipping in solutions of 0.75% potassium permanganate (KMnO₄) and applying heat. Flash chromatography was performed with Dynamic Adsorbents, Inc. silica gel (60, particle size 0.040-0.063 mm). Deuterated solvents were purchased from Cambridge Isotope Laboratories, Inc. ¹H and ¹³C NMR spectra were obtained on either Varian 400 MHz/500 MHz or JEOL 400 MHz or Bruker 800 MHz and calibrated using the residual non-deuterated solvent as an internal reference. The following abbreviations were used to explain the multiplicities: s=singlet, d=doublet, t=triplet, q=quartet, m=multiplet, br=broad. Low resolution MS analysis was performed on a Micromass Quattro Ultima triple quadrupole mass spectrometer with an electrospray ionization (ESI) source. High resolution MS analysis was performed using Agilent 6230 Accurate-Mass TOFMS with an electrospray ionization (ESI) source by Molecular Mass Spectrometry Facility (MMSF) in the Department of Chemistry and Biochemistry at University of California, San Diego. Fluorescence characterization was performed using a Molecular Devices SpectraMax i3x Multi-Mode Microplate Reader. HPLC characterization of all final probes was conducted using the Agilent 1260 Infinity II Quaternary Pump System, where 10 μL of probe at a final concentration of 200 μM (2.5% DMSO/H₂O) was injected into a 150 mm×3 mm, 2.7 m particle size C18 column (693975-302T). Solvent conditions (Solvent A: H₂O (0.1% TFA), Solvent B: Acetonitrile): 2 min 5% B, 20 min 60% B, 22 min 60% B, 24 min 0% B, 30 min 0% B at 1 m1/min.

### Procedures for Fluorescent Probe 4 Synthesis

### General Synthetic Procedures:

### Buchwald-Hartwig Coupling:

To a solution of Pd(OAc)₂ (0.1 eq) in degassed anhydrous toluene (0.026 M) in a microwave vial, rac-BINAP (0.1 eq) was added. The solution was degassed and stirred for 20 minutes at room temperature. After, 6-bromo-2-naphthaldehyde (1 eq), Cs₂CO₃ (1.34 eq), and amine (1.2 eq) were added. The solution was degassed, the vial sealed, and the mixture was left stirring overnight at reflux. Upon completion, DCM was added and the mixture was filtered through a pad of celite and purified via silica column chromatography.

### Knoevenagel Condensation:

To a solution of aryl aldehyde (1 eq) in anhydrous THE (0.29 M) in a microwave tube was added 2-cyano-N-(2-(2-(2-methoxyethoxy)ethoxy)ethyl)acetamide (1.2 eq) and piperidine (0.01 eq). The tube was sealed, heated to 50° C. under inert atmosphere, and left stirring overnight. Upon completion, the reaction mixture was cooled to room temperature and concentrated under reduced pressure, and purified via silica column chromatography.

### Suzuki Coupling:

To a solution of starting bromide (1 eq) in a mixture of 1,4-dioxane/water (1:1) (0.43 M) was added 4-formylboronic acid (1.3 eq) and potassium carbonate (4.5 eq). The reaction mixture was stirred at room temperature for 30 minutes, after which bis(triphenylphosphine) palladium chloride (0.05 eq) was added and degassed. The mixture was heated at 80° C. overnight, cooled to room temperature, and evaporated under reduced pressure. The crude mixture was extracted with ethyl acetate, washed with brine, dried with sodium sulfate, and concentrated under reduced pressure. Purified under silica column chromatography.

### Horner-Wadsworth-Emmons Reaction:

To a solution of benzaldehyde (1 eq) in anhydrous THE (0.026 M) was added diethyl(4-bromobenzyl)phosphonate (1 eq) and potassium tert-butoxide (1.5 eq). The reaction mixture was stirred at room temperature for 2 hours, quenched with water, and concentrated under reduced pressure. The resulting mixture was extracted with DCM, dried with sodium sulfate, filtered, concentrated under reduced pressure, and purified under silica column chromatography.

### Formylation Reaction:

A solution of stilbene bromide (1 eq) in anhydrous THE (0.029 M) was cooled down to −40° C. under inert atmosphere. A solution of n-BuLi (2.5 M in hexanes, 1.2 eq) was added dropwise, and the mixture was stirred for one hour, upon which dimethylformamide (1.67 eq) was added and stirred for 2 hours. The reaction mixture was warmed to room temperature, quenched with saturated ammonium chloride, extracted with DCM, and concentrated under reduced pressure. Purified under silica column chromatography.

### Synthetic Procedure for NAPHTHCAM-Pr

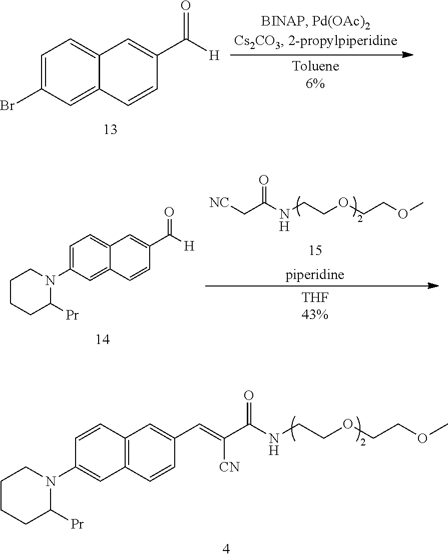

### 6-(2-propylpiperidin-1-yl)-2-naphthaldehyde (14)

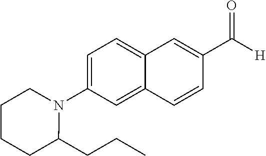 [SMILES: CCCC1CCCCN1c1ccc2cc(C=O)ccc2c1]

Aldehyde (14) was synthesized from 6-bromo-2-naphthaldehyde³¹ (337 mg, 1.4 mmol) and 2-propylpiperidine (0.26 mL, 1.7 mmol) according to the general procedure for Buchwald-Hartwig Coupling (see above). 14 (22 mg, 6%) was obtained as a yellow solid after purification by column chromatography on silica gel using hexane/EtOAc (90:10) as the eluent, followed by a plug of 100% DCM.

Rf=0.21 (hexane/EtOAc 92.5:7.5); ¹H NMR (500 MHz, CDCl₃) δ 10.01 (s, 1H), 8.13 (s, 1H), 7.80 (t, J=10.0 Hz, 2H), 7.63 (d, J=8.6 Hz, 1H), 7.30 (dd, J=9.2, 2.6 Hz, 1H), 7.01 (d, J=2.5 Hz, 1H), 4.12 (d, J=4.5 Hz, 1H), 3.66 (dd, J=13.2, 3.8 Hz, 1H), 3.12 (t, J=12.4 Hz, 1H), 1.83-1.70 (m, 3H), 1.70-1.60 (m, 5H), 1.38-1.22 (m, 2H), 0.89 (t, J=7.3 Hz, 3H); ¹³C NMR (126 MHz, CDCl₃) δ 192.09, 151.56, 138.91, 134.69, 131.01, 130.73, 127.08, 125.76, 123.53, 118.83, 108.29, 54.96, 42.43, 30.35, 27.75, 25.45, 20.33, 18.93, 14.32; ESI-MS: 282.32 [M+H]⁺.

### (E)-2-cyano-N-(2-(2-(2-methoxyethoxy)ethoxy)ethyl)-3-(6-(2-propylpiperidin-1-yl)naphthalen-2-yl)acrylamide (4)

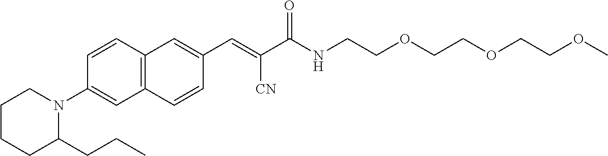 [SMILES: CCCC1CCCCN1c1ccc2cc(/C=C(\C#N)C(=O)NCCOCCOCCOC)ccc2c1]

4 was synthesized from 14 (29 mg, 0.1 mmol) and 15 (28 mg, 0.12 mmol) according to the general procedure for the Knoevenagel Condensation (see above). 4 (21 mg, 43%) was obtained as a yellow solid after purification by column chromatography on silica gel using hexane/EtOAc (10:90) as the eluent. Compound was 99% pure by HPLC as a mixture of 1:2.5 E:Z Isomers.

Rf=0.52 (100% EtOAc); ¹H NMR (500 MHz, Chloroform-d) δ 8.35 (s, 1H), 8.16-8.12 (m, 1H), 8.03 (dd, J=8.7, 1.9 Hz, 1H), 7.73 (d, J=9.2 Hz, 1H), 7.57 (dd, J=33.5, 8.8 Hz, 1H), 7.31-7.22 (m, 1H), 7.00-6.92 (m, 1H), 6.88 (t, J=4.8 Hz, 1H), 4.15-4.06 (m, 1H), 3.67-3.65 (m, 10H), 3.61-3.55 (m, 1H), 3.53-3.44 (m, 1H), 3.38 (s, 3H), 3.32 (s, 1H), 3.11 (t, J=11.9 Hz, 1H), 1.91 (s, 2H), 1.80-1.71 (m, 4H), 1.71-1.50 (m, 4H), 0.87 (q, J=6.8, 6.3 Hz, 3H); ¹³C NMR (101 MHz, CHLOROFORM-D) δ 161.50, 153.17, 151.40, 137.69, 134.05, 130.64, 127.06, 126.12, 125.88, 125.72, 118.83, 118.09, 108.12, 100.03, 72.03, 70.72, 70.60, 70.41, 69.58, 59.15, 54.92, 42.41, 40.31, 29.80, 27.75, 25.41, 20.27, 18.89, 14.26; HRMS 494.3013 calcd for [C₂₉ H₄₀N₃ O₄]⁺, found 494.3010.

### Molecular Docking

All steps for the docking experiments were carried out using Schrodinger suite 2020-21. The solid-state NMR structures of Aβ fibril (PDB ID: 2MXU) and full length αS fibril (PDB ID: 2NOA) were retrieved from RCSB Protein Data Bank. The proteins were prepared using Maestro's Protein Preparation Wizard. SiteMap tool was used to identify probable binding sites on both the amyloid proteins. For Aβ, only one major probable site was identified which included residues 12, 14, 17, 32, 33, 34. For αS, a total of 4 sites were identified; Site 1 residues—1, 2, 3, 4, 43, 45, 48, 50; Site 2 residues—39, 42, 43, 44; Site 3 residues—85, 86, 87, 88, 94, 96; Site 4 residues—59, 61, 64, 70, 72, 73. The receptor grids were then generated at the center of mass of the pocket residues and was made large enough to contain the binding site of the protein and the surrounding surface regions. All the ligands in the study were constructed in Maestro and LigPrep was used minimize individual structures to prepare the ligand for molecular docking. Molecular docking was carried out using Glide with extra precision (XP) docking into each binding site with default parameters to generate 100 poses for the ligand-receptor complex. The poses were clustered together using a cutoff RMSD value of 2 A. Molecular Operating Environment (MOE) software was used to study the top poses and expected interactions between ligand and protein residues in the binding sites. All the docked pose images were generated using Chimera.

TABLE 2
Estimated Maximum Lengths for Substituents

| | Maximum |
|---|---|
| Substituent | Length (Å) |
| Core | 1.46 |
| H | 2.55 |
| Me | 3.00 |
| Et | 4.54 |
| Pr | 6.08 |

TABLE 3
Estimated Distances from Piperidine
Core to Binding Pocket Residues

| | | Distance |
|---|---|---|
| Amyloid | Residues | (Å) |
| Aβ | Ile32 | 3.37 |
| | Gly33 | 4.30 |
| | Leu34 | 4.90 |
| | Leu17 | 5.83 |
| | His14 | 7.00 |
| | Val12 | 10.50 |
| αS | Gly86 | 3.14 |
| | Ile88 | 4.08 |
| | Phe94 | 4.30 |
| | Lys96 | 4.01 |

### Quantum Yield Measurements

Quantum Yield ((D) for each probe was measured as described previously.³¹ Briefly, we measured the absorbance spectrum of each probe in 5% DMSO/H₂O, as well as the absorbance spectrum for the reference standard (Coumarin 30, =0.67 in Acetonitrile). Both normalized absorbance curves were plotted, and the wavelength of intersection was used as the excitation wavelength. A serial dilution was made for each probe and the standard with absorbance values at the excitation wavelength varying between of 0 to 0.1 absorbance units. At each concentration, the emission spectra for each probe and the standard were measured. Each experiment was repeated in technical triplicates for all probes, and the averages were recorded. The integrated fluorescence emission was calculated and plotted against the absorbance at each concentration. The data were fitted to Equation Si to obtain estimates for Φ:

$${Φ}_{p} = {Φ}_{r} ( \frac{{A}_{r}}{{A}_{p}} ) ( \frac{{E}_{p}}{{E}_{r}} ) {( \frac{{η}_{p}}{{η}_{r}} )}^{2} \qquad ( 1 )$$

where Φ represents the quantum yield, A represents the absorbance, E represents the integrated fluorescence emission, η represents the refractive index of the solvent, and the ‘p’ and ‘r’ subscripts signify the probe or the reference compound, respectively.

### Preparation of Aggregated Aβ (1-42) Peptides

Synthetic Aβ (1-42) peptide was purchased from Biopeptide, Co LLC (San Diego, California). Aggregated Aβ (1-42) was prepared as previously described.³² Briefly, monomeric Aβ (1-42) was dissolved in 100% 1,1,1,3,3,3,-hexafluoro-2-propanol (HFIP) to a final concentration of 1 mM and dissolved on a shaker at RT for 24 hours. This solution was then diluted with cold nanopure water (2:1 H₂O:HFIP) and aliquoted into fractions. These samples were then lyophilized for 3 days before dissolving in nanopure water to a final concentration of 100 μM. AD solutions were aggregated by incubating and shaking at 37° C. for 3 days before use. The formation of soluble aggregates was confirmed using a standard Thioflavin T (ThT) assay and by SDS-PAGE gel analysis.¹

### Expression, Purification, and Aggregated of αS Protein

Expression and purification of recombinant α-synuclein was followed as reported previously.³² Briefly, α-synuclein was purified from BL21 E. coli as described: A starter culture of transformed bacteria (pET5α aSynWT) (LB broth with ampicillin [final concentration: 100 μg/mL]) was incubated overnight. Following incubation, 25 mL of the started culture was added to a 500 mL mixture of LB broth and ampicillin. Protein expression was induced by addition of IPTG [0.119 g/500 mL] when an OD₆₀₀ between 0.5-0.8 was achieved. After 4 h, flasks were centrifuged (5,000×g for 20 min) and the supernatant was removed. The pellets were then re-suspended in 20 mM Tris, 25 mM NaCl, 1 mM EDTA, pH 8.0, transferred into a flask, boiled for 1-2 minutes in a microwave, and boiled in a water bath (90° C.) for 30 minutes. Samples were then centrifuged (18,000×g for 30 min) and the supernatant was collected and filtered through a 0.2 μm filter. α-synuclein protein was further purified using 2×5 ml HiTrapQ HP anion exchange columns (GE, #17-1153-01) followed by Size Exclusion Column (Superdex 200, #28-9893-35). Pure α-synuclein determined by Coomassie and western blot were lyophilized and stored at −80° C. until use.

Aggregation of purified α-synuclein was followed as reported previously.³³ The lyophilized powder was dissolved to a final concentration of 100 μM in sodium phosphate buffer (50 mM, pH 7.4) with 0.1 mM EDTA and 0.02% sodium azide (w/v) and was aggregated at 37° C. with shaking at 500 rpm for 3 weeks. Fibrillization was confirmed by SDS-PAGE gel and standard ThT assay.

### Fluorescence Excitation and Emission Spectra Both Unbound and Bound to Aggregated αS and Aβ (1-42)

Aggregated αS or Aβ(1-42) at a final concentration of 5 μM (based on the molecular weight of monomer) was mixed with each fluorescent probe at a final concentration of 4 μM in 5% DMSO in nanopure water. The solution (80 μL total volume) was transferred to a black opaque 96 well plate and the fluorescence excitation and emission were read using a microplate reader. For emission fluorescence enhancement, the relative fluorescence enhancement of the bound versus unbound state was estimated by comparing the fluorescence intensity at λₘₐₓ of the amyloid-bound state.

Estimation of Binding Constant (K_{d}) to aggregated αS and Aβ(1-42)

Aggregated Aβ(1-42) or αS at a final concentration of 5 μM (based on the molecular weight of the monomer) was mixed with an increasing concentration of each probe in 5% DMSO in nanopure water. K_{d}'s were calculated as previously described.³⁴ The measurements for each probe were repeated in technical triplicates and the averages are shown with standard deviations. K_{d}'s for probes 1-3 with aggregated Aβ(1-42) have been reported previously.³¹

Tissue Staining with Fluorescent Probes

Fresh, frozen samples of brain were obtained from the UCSD Alzheimer's Disease Research Center (ADRC) in accordance with the UC San Diego IRB for human research. Tissue donors were psychometrically and neurologically studied at the Shiley-Marcos ADRC at UC San Diego. Upon the post-mortem autopsy, patient brains were collected by the UCSD ADRC Neuropathology Core. The AD human brain was from the frontal cortex of an 87 year old female patient diagnosed with Braak Stage 6 AD. Samples from the PD human brain were from the frontal cortex, hippocampus, and substantia nigra of a 68 year old male patient diagnosed with Braak Stage 1 PD with dementia. The control human brain was from an 80 year old female patient with no known neurodegenerative disease pathology.

Sections of thickness 10 μm were obtained from a cryostat kept at −20° C. and stored at −80° C. until use. Each section was warmed to 37° C. for one hour, followed by hydrating the tissue 2×10 minutes with 1×PBS. The sections were then fixed for 10 minutes with 4% paraformaldehyde solution, washed 2×5 minutes with 1×PBS, 1×5 minutes with diH₂0, and hydrated 2×10 minutes with 1×PBS.* The tissue sections were permeabilized with 0.1% Triton-X (in 1×PBS) for 1×15 minutes, followed by a 30-minute incubation of each probe (final concentration 30 μM in 2% DMSO in 1×PBS) in the dark. The remaining probe was washed 3×5 minutes with 1×PBS and mounted with DAKO mounting media.

### Tissue Staining for Immunohistochemistry

The brain tissue samples for immunohistochemistry were treated as previously described above through the hydration step (*). Following this step, they were treated for antigen retrieval with 88% Formic Acid for 5 minutes, which was followed by a 5-minute washing step with diH2O and 3×5 minute washing steps with 1×PBS. The tissue was permeabilized with 0.1% Triton-X (in 1×PBS) for 1×15 minutes, followed by a one-hour blocking step with TNB buffer. This was followed by incubation of the primary antibody (auS: SYN1, BD Biosciences Cat. 610786, dilution: 1:500; Aβ: 6e10, Biolegend Cat. 803001, dilution 1:500) at 4° C. overnight. The next day, the primary antibody was washed with 0.1% Triton-X for 5×5 minutes, followed by incubating the tissue with the secondary antibody (Alexa Fluor 647, dilution 1:500) for one hour at room temperature. The remaining secondary antibody was washed off with 1×PBS for 5×5 minutes, followed by mounting with DAKO mounting media.

### Spectral Scans

Each tissue sample was excited using an argon 488 nm laser on an Olympus FluoView FV1000 confocal microscope. The emission spectra of probes bound to αS, Aβ, or background tissue were collected in 5 nm increments from 470-700 nm. A minimum of 5 measurements were collected for each probe bound to each amyloid and for background. The signal to background was calculated by dividing the peak intensity for the bound probe by an average of the background measurements at the maximum wavelength of the bound probe.

TABLE 4
Fluorescence Profile, Fold Increase (FI), Ka, and Quantum
Yield of the Synthesized Probes 1-4 from FIG. 14

| | | Emₘₐₓ | Emₘₐₓ | | | | | | |
|---|---|---|---|---|---|---|---|---|---|
| | Emₘₐₓ | (with | (with | | | FI | K_{d} with | | |
| Cmpd | (Free) | αS) | Aβ) | FI | FI | αS/ | αS | K_{d} with | Quantum |
| no. | (nm) | (nm) | (nm) | with αS | with Aβ | Aβ | (μM) | Aβ (μM) | Yield (Φ) |
| 1 | 590 | 539 | 560 | 3.0 ± 0.5 | 2.1 ± 0.7 | 1.4 | 3.9 ± 3.9 | 6.3 ± 0.7 | 0.0066 ± |
| | | | | | | | | | 0.0023 |
| 2 | 583 | 531 | 559 | 3.8 ± 0.4 | 1.9 ± 0.5 | 2.0 | 4.5 ± 1.6 | 4.4 ± 2.3 | 0.0062 ± |
| | | | | | | | | | 0.0003 |
| 3 | 575 | 534 | 567 | 4.6 ± 0.7 | 1.2 ± 0.1 | 3.8 | 5.8 ± 1.8 | 3.2 ± 0.6 | 0.0055 ± |
| | | | | | | | | | 0.0010 |
| 4 | 578 | 539 | 558 | 5.2 ± 0.8 | 1.8 ± 0.2 | 2.9 | 0.5 ± 0.1 | 4.0 ± 2.7 | 0.0062 ± |
| | | | | | | | | | 0.0002 |

### REFERENCES

1. Alzheimer's Association. 2021 Alzheimer's Disease Facts and Figures. Alzheimer's Dement. 2021, 17 (3), 327-406. 2. Alzheimer's Association. 2015 Alzheimer's Disease Facts and Figures. Alzheimer's Dement. 2015, 11 (3), 332-384. 3. Walsh, D. M.; Selkoe, D. J. Deciphering the Molecular Basis of Memory Failure in Alzheimer's Disease. Neuron 2004, 44 (1), 181-193. 4. Chiti, F.; Dobson, C. M. Protein Misfolding, Amyloid Formation, and Human Disease: A Summary of Progress Over the Last Decade. Annu. Rev. Biochem. 2017, 86 (1), 27-68. 5. Nordberg, A. Amyloid Imaging in Alzheimer's Disease. Curr. Opin. Neurol. 2007, 20 (4), 398-402. 6. Xu, M. M.; Ryan, P.; Rudrawar, S.; Quinn, R. J.; Zhang, H. Y.; Mellick, G. D. Advances in the Development of Imaging Probes and Aggregation Inhibitors for Alpha-Synuclein. Acta Pharmacol. Sin. 2020, 41 (4), 483-498. 7. Perani, D.; Iaccarino, L.; Sala, A.; Caminiti, S. P. The Emerging Role of PET Imaging in Dementia. F1000Research 2017, 6 (1830), 1-13. 8. Manook, A.; Yousefi, B. H.; Willuweit, A.; Platzer, S.; Reder, S.; Voss, A.; Huisman, M.; Settles, M.; Neff, F.; Velden, J.; et al. Small-Animal PET Imaging of Amyloid-Beta Plaques with [11 C] PiB and Its Multi-Modal Validation in an APP/PS1 Mouse Model of Alzheimer's Disease. PLoS One 2012, 7 (3), 1-23. 9. Kerbage, C.; Sadowsky, C. H.; Jennings, D.; Cagle, G. D.; Hartung, P. D. Alzheimer's Disease Diagnosis by Detecting Exogenous Fluorescent Signal of Ligand Bound to Beta Amyloid in the Lens of Human Eye: An Exploratory Study. Front. Neurol. 2013, 4 (62), 1-9. 10. Sadowsky, C. H.; Kerbage, C.; Tariot, P. N.; Alva, G.; Doraiswamy, P. M.; Darell, F.; Nilan, D.; Cameron, A.; Cagle, G. D.; Hartung, P. D. Diagnosis of Alzheimer's Disease through the Eye and Its Correlation with Cognitive Tests and Brain Imaging. JMS Alzheimer's Dis. Relat. Dement. 2014, 1 (2), 1-5. 11. Kerbage, C.; Sadowsky, C. H.; Tariot, P. N.; Agronin, M.; Alva, G.; Turner, F. D.; Nilan, D.; Cameron, A.; Cagle, G. D.; Hartung, P. D. Detection of Amyloid β Signature in the Lens and Its Correlation in the Brain to Aid in the Diagnosis of Alzheimer's Disease. Am. J. Alzheimers. Dis. Other Demen. 2015, 30 (8), 738-745. 12. Xu, M. M.; Ren, W. M.; Tang, X. C.; Hu, Y. H.; Zhang, H. Y. Advances in Development of Fluorescent Probes for Detecting Amyloid-β Aggregates. Acta Pharmacol. Sin. 2016, 37 (6), 719-730. 13. Fu, H.; Cui, M. Fluorescent Imaging of Amyloid-β Deposits in Brain: An Overview of Probe Development and a Highlight of the Applications for In Vivo Imaging. Curr. Med. Chem. 2018, 25 (23), 2736-2759. 14. Cao, K. J.; Elbel, K. M.; Cifelli, J. L.; Cirera, J.; Sigurdson, C. J.; Paesani, F.; Theodorakis, E. A.; Yang, J. Solvation-Guided Design of Fluorescent Probes for Discrimination of Amyloids. Sci. Rep. 2018, 8 (1), 1-13. 15. Sutharsan, J.; Dakanali, M.; Capule, C. C.; Haidekker, M. A.; Yang, J.; Theodorakis, E. A. Rational Design of Amyloid Binding Agents Based on the Molecular Rotor Motif. ChemMedChem 2010, 5 (1), 56-60. 16. Stsiapura, V. I.; Maskevich, A. A.; Kuzmitsky, V. A.; Uversky, V. N.; Kuznetsova, I. M.; Turoverov, K. K. Thioflavin T as a Molecular Rotor: Fluorescent Properties of Thioflavin T in Solvents with Different Viscosity. J. Phys. Chem. B 2008, 112 (49), 15893-15902. 17. Sutharsan, J.; Lichlyter, D.; Wright, N. E.; Dakanali, M.; Haidekker, M. A.; Theodorakis, E. A. Molecular Rotors: Synthesis and Evaluation as Viscosity Sensors. Tetrahedron 2010, 66 (14), 2582-2588. 18. Lakowicz, J. R. Principles of Fluorescence Spectroscopy, 3rd ed.; Springer Science+Business Media, LLC: New York, NY, 2006. 19. Chang, W. M.; Dakanali, M.; Capule, C. C.; Sigurdson, C. J.; Yang, J.; Theodorakis, E. A. ANCA: A Family of Fluorescent Probes That Bind and Stain Amyloid Plaques in Human Tissue. ACS Chem. Neurosci. 2011, 2 (5), 249-255. 20. Cao, K.; Farahi, M.; Dakanali, M.; Chang, W. M.; Sigurdson, C. J.; Theodorakis, E. A.; Yang, J. Aminonaphthalene 2-Cyanoacrylate (ANCA) Probes Fluorescently Discriminate between Amyloid-β and Prion Plaques in Brain. J. Am. Chem. Soc. 2012, 134 (42), 17338-17341. 21. Guan, Y.; Cao, K. J.; Cantlon, A.; Elbel, K.; Theodorakis, E. A.; Walsh, D. M.; Yang, J.; Shah, J. V. Real-Time Monitoring of Alzheimer's-Related Amyloid Aggregation via Probe Enhancement-Fluorescence Correlation Spectroscopy. ACS Chem. Neurosci. 2015, 6 (9), 1503-1508. 22. Do, J. P.; Cao, K. J.; Wei, S.; Laurent, L. C.; Parast, M. M.; Yang, J. Identification of Patients with Preeclampsia by Measuring Fluorescence of an Amyloid-Binding Aryl Cyano Amide in Human Urine Samples. Anal. Chem. 2018, 90 (24), 14316-14320. 23. Cao, K. J.; Kim, J. H.; Kroeger, H.; Gaffney, P. M.; Lin, J. H.; Sigurdson, C. J.; Yang, J. ARCAM-1 Facilitates Fluorescence Detection of Amyloid-Containing Deposits in the Retina. Transl. Vis. Sci. Technol. 2021, accepted for publication. 24. Freire, S.; De Araujo, M. H.; Al-Soufi, W.; Novo, M. Photophysical Study of Thioflavin T as Fluorescence Marker of Amyloid Fibrils. Dye. Pigment. 2014, 110, 97-105. 25. Sinkeldam, R. W.; Tor, Y. To D or Not to D?: On Estimating the Microenvironment Polarity of Biomolecular Cavities. Org. Biomol. Chem. 2007, 5 (16), 2523-2528. 26. Cifelli, J. L.; Capule, C. C.; Yang, J. Noncovalent, Electrostatic Interactions Induce Positively Cooperative Binding of Small Molecules to Alzheimer's and Parkinson's Disease-Related Amyloids. ACS Chem. Neurosci. 2019, 10 (2), 991-995. 27. Jones, G.; Jackson, W. R.; Choi, C. Y.; Bergmark, W. R. Solvent Effects on Emission Yield and Lifetime for Coumarin Laser Dyes. Requirements for a Rotatory Decay Mechanism. J. Phys. Chem. 1985, 89 (2), 294-300. 28. Lebel, R. G.; Goring, D. A. I. Density, Viscosity, Refractive Index, and Hygroscopicity of Mixtures of Water and Dimethyl Sulfoxide. J. Chem. Eng. Data 1962, 7 (1), 100-101. 29. Moutzouris, K.; Papamichael, M.; Betsis, S. C.; Stavrakas, I.; Hloupis, G.; Triantis, D. Refractive, Dispersive and Thermo-Optic Properties of Twelve Organic Solvents in the Visible and near-Infrared. Appl. Phys. B Lasers Opt. 2014, 116 (3), 617-622. 30. Cabaret, D.; Welvart, Z.; Carrie, R. Influence de l'isomerie Cis-Trans Des Alcoylidene-Cyanacetates Sur Leur Reactivite Vis-a-Vis Des Reactifs Organomagnesiens. J. Organomet. Chem. 1974, 70 (2), 229-238. 31. Ehrlich, R. S.; Shiao, A. L.; Li, M.; Teppang, K. L.; Jeoung, K. Y.; Theodorakis, E. A.; Yang, J. Exploring the Effect of Aliphatic Substituents on Aryl Cyano Amides on Enhancement of Fluorescence upon Binding to Amyloid-β Aggregates. ACS Chem. Neurosci. 2021, 12 (15), 2946-2952. https://doi.org/10.1021/ACSCHEMNEURO.1C_{00334. 32}. Cifelli, J. L.; Capule, C. C.; Yang, J. Noncovalent, Electrostatic Interactions Induce Positively Cooperative Binding of Small Molecules to Alzheimer's and Parkinson's Disease-Related Amyloids. ACS Chem. Neurosci. 2019, 10 (2), 991-995. 33. Tuttle, M. D.; Comellas, G.; Nieuwkoop, A. J.; Covell, D. J.; Berthold, D. A.; Kloepper, K. D.; Courtney, J. M.; Kim, J. K.; Barclay, A. M.; Kendall, A.; et al. Solid-State NMR Structure of a Pathogenic Fibril of Full-Length Human α-Synuclein. Nat. Struct. Mol. Biol. 2016235 2016, 23 (5), 409-415. https://doi.org/10.1038/nsmb.3194. 34. Do, J. P.; Cao, K. J.; Wei, S.; Laurent, L. C.; Parast, M. M.; Yang, J. Identification of Patients with Preeclampsia by Measuring Fluorescence of an Amyloid-Binding Aryl Cyano Amide in Human Urine Samples. Anal. Chem. 2018, 90 (24), 14316-14320.

1. A compound having the formula: 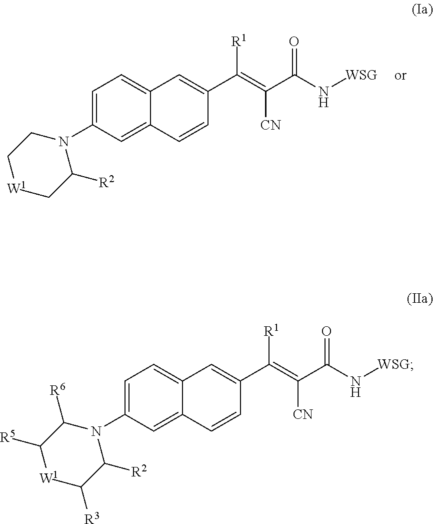
or a pharmaceutically acceptable salt thereof;
wherein:
WSG is a water soluble group;
W¹ is O, N(R¹⁴), or C(R⁴)₂;
R¹⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹, R², R³, R⁴, R⁵, and R⁶ are each independently hydrogen, halogen, —CX¹₃, —CHX¹₂, —CH₂X¹, —OCX¹₃, —OCH₂X¹, —OCHX¹₂, —SOₙ₁R^{1A}, —SOᵥ₁NR^{1A}R^{1B}, —CN, —C(O)R^{1A}, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, —NHNR^{1A}R^{1B}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are each independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹ is —F, —Cl, —Br, or —I;
n1 is independently an integer from 0 to 4;
m1 is independently 1 or 2; and
v1 is independently 1 or 2.

2. The compound of claim 1, having the formula:

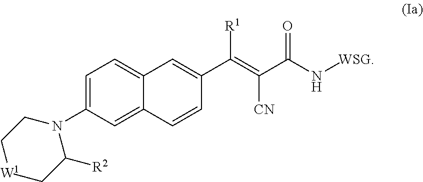

3. The compound of claim 1, having the formula:

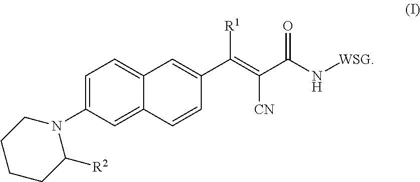

4. The compound of claim 2, wherein R¹ is hydrogen.

5. The compound of claim 2, wherein R² is hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH—₂, —NHNH₂, —ONH₂, —NHC(O)NHNH₂, substituted or unsubstituted C₁-C₆ alkyl, or substituted or unsubstituted 2 to 6 membered heteroalkyl.

6. (canceled)

7. (canceled)

8. The compound of claim 2, wherein R³, R⁴, R⁵, and R⁶ are hydrogen.

9. The compound of claim 1, having the formula:

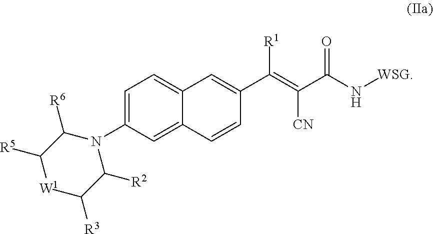

10. The compound of claim 1, having the formula:

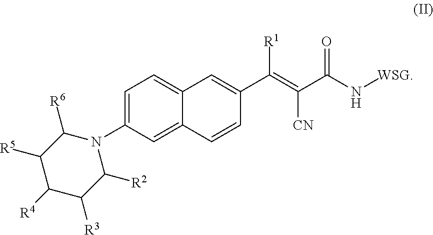

11. The compound of claim 9, wherein R¹ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted C₁-C₆ alkyl, or substituted or unsubstituted 2 to 6 membered heteroalkyl.

12. (canceled)

13. (canceled)

14. The compound of claim 9, wherein R², R³, R⁴, R⁵, and R⁶ are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH—₂, —NHNH₂, —ONH₂, —NHC(O)NHNH₂, substituted or unsubstituted C₁-C₆ alkyl, or substituted or unsubstituted 2 to 6 membered heteroalkyl.

15. The compound of claim 9, wherein R², R³, R⁴, R⁵, and R⁶ are hydrogen.

16.-18. (canceled)

19. The compound of claim 1, wherein WSG is 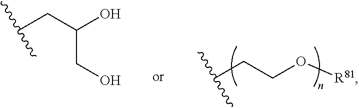
wherein n is an integer from 1 to 50 and R⁸¹ is hydrogen, substituted or unsubstituted C₁-C₁₀ alkyl, substituted or unsubstituted C₂-C₁₀ alkenyl, or substituted or unsubstituted C₂-C₁₀ alkynyl.

20. (canceled)

21. (canceled)

22. The compound of claim 1, wherein WSG is

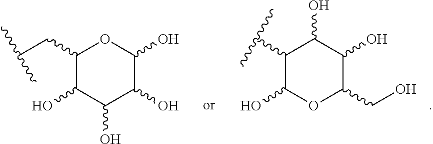

23. (canceled)

24. The compound of claim 1, wherein WSG is -(unsubstituted C₁-C₁₀ alkyl)-R³³—R³⁷, wherein
R³³ is unsubstituted 5 to 10 membered heteroarylene; and
R³⁷ is -(unsubstituted C₁-C₆ alkyl)-(unsubstituted 5 to 10 membered heterocycloalkyl).

25. The compound of claim 1, wherein WSG is 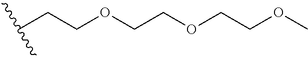 [SMILES: *CCOCCOCCOC]
or —CH₂OCH₂CH₂OCH₂CH₂OCH₃.

26. (canceled)

27. A pharmaceutical composition comprising the compound of claim 1, and a pharmaceutically acceptable excipient.

28. (canceled)

29. A composition comprising the compound of claim 1, and an amyloid or amyloid like protein.

30. (canceled)

31. A method of detecting an amyloid or amyloid like protein comprising (a) contacting the compound of claim 1, with a sample potentially comprising the amyloid or amyloid like protein, wherein in presence of an amyloid or amyloid like protein the compound forms a detectable complex; and
(b) detecting the formation of the detectable complex such that the presence or absence of the detectable complex correlates with the presence or absence of the amyloid or amyloid like protein.

32.-40. (canceled)

41. A method of determining the presence or absence of one or more disease or condition in a subject comprising
(a) administering to the subject an effective amount of the compound of claim 1, or a pharmaceutical composition thereof, wherein in presence of the disease or condition the administered compound forms a detectable complex; and
(b) detecting the formation of the detectable complex such that presence or absence of detectable complex correlates with the presence or absence of the disease or condition.

42. (canceled)

43. (canceled)

44. The method of claim 41, wherein the disease is Alzheimer's disease, Parkinson's disease, Huntington's disease, amyotrophic lateral sclerosis, Lewy body dementia, or Down's syndrome.
